(19) United States
(12) Patent Application Publication
(10) Pub. No.: US 2023/0027007 A1
(43) Pub. Date: Jan. 26, 2023

(54) Treatment of Hypertension With Solute Carrier Family 9 Isoform A3 Regulatory Factor 2 (SLC9A3R2) Inhibitors

(71) Applicant: Regeneron Pharmaceuticals, Inc., Tarrytown, NY (US)

(72) Inventors: Manuel Allen Revez Ferreira, Tarrytown, NY (US); Joshua Backman, Tarrytown, NY (US); Alexander Li, Tarrytown, NY (US); Luca Andrea Lotta, Tarrytown, NY (US); Goncalo Abecasis, Tarrytown, NY (US); Aris Baras, Tarrytown, NY (US); Jonas Nielsen, Tarrytown, NY (US)

(21) Appl. No.: 17/854,589

(22) Filed: Jun. 30, 2022

Related U.S. Application Data
(60) Provisional application No. 63/217,909, filed on Jul. 2, 2021.
(60) Provisional application No. 63/221,031, filed on Jul. 13, 2021.

(51) Int. Cl. C12N 15/113; A61K 45/06; A61P 9/12

(52) U.S. Cl. CPC C12N 15/113; A61K 45/06; A61P 9/12; C12N 2310/14; C12N 2310/531

(57) ABSTRACT

The present disclosure provides methods of treating subjects having hypertension, coronary heart disease, and/or atrial fibrillation or at risk of developing hypertension, coronary heart disease, and/or atrial fibrillation, methods of identifying subjects having an increased risk of developing hypertension, coronary heart disease, and/or atrial fibrillation, and methods of detecting Solute Carrier Family 9 Isoform A3 Regulatory Factor 2 (SLC9A3R2) variant nucleic acid molecules and variant polypeptides.

### REFERENCE TO SEQUENCE LISTING

This application includes a Sequence Listing submitted electronically as a text file named 18923807901SEQ, created on Jun. 25, 2022, with a size of 225 kilobytes. The Sequence Listing is incorporated herein by reference.

### FIELD

The present disclosure relates generally to the treatment of subjects having hypertension, coronary heart disease, and/or atrial fibrillation or at risk of developing hypertension, coronary heart disease, and/or atrial fibrillation with Solute Carrier Family 9 Isoform A3 Regulatory Factor 2 (SLC9A3R2) inhibitors, and methods of identifying subjects having an increased risk of developing hypertension, coronary heart disease, and/or atrial fibrillation.

### BACKGROUND

Hypertension is the most common of all cardiovascular diseases afflicting about 10% to 20% of the adult population. High blood pressure together with the consequential illnesses thereof (arteriosclerosis, cardiac infarction, strokes, cardiac hypertrophy and cardiac insufficiency) represents the most frequent cause of illness and death in all Western industrial nations, even ahead of malignant degenerative illnesses (cancer). High blood pressure is due to excessive constriction of the blood vessels or inadequate excretion of fluid by the kidneys. A large number of central-nervous mechanisms and hormone systems are involved in regulation of the muscle tone of the smooth musculature in the blood vessels and, thus, the vessel width and also in fluid excretion by the kidneys. The mechanisms and hormone systems referred to above control and regulate the blood pressure which rises physiologically in relation to physical work, fear, stress, excitement and so forth. Derailment of one or more of those systems ultimately results in high blood pressure. In many instances, the true causes of high blood pressure are unknown (essential hypertonia). Genetic predisposition due to mutation of genes which code for proteins which are involved in blood pressure-regulating systems, sometimes only in combination with external factors (stress, smoking, overweight, lack of physical movement, poor diet) is however highly probable.

SLC9A3R2 is a member of the Na⁺—H⁺ exchanger regulatory factor (NHERF) family of PDZ (PSD-95/DLG/ZO-1) scaffolding proteins. These proteins mediate many cellular processes by binding to and regulating the membrane expression and protein-protein interactions of membrane receptors and transport proteins. SLC9A3R2 is expressed in kidney and connects plasma membrane proteins with members of the ezrin/moesin/radixin family and thereby helps to link them to the actin cytoskeleton and to regulate their surface expression. SLC9A3R2 also plays a role in intestinal sodium absorption by regulating the activity of the sodium/hydrogen exchanger 3, and may also regulate the cystic fibrosis transmembrane regulator (CFTR) ion channel, and is necessary for cAMP-mediated phosphorylation and inhibition of SLC9A3.

### SUMMARY

The present disclosure provides methods of treating a subject having hypertension or at risk of developing hypertension, the methods comprising administering an SLC9A3R2 inhibitor to the subject.

The present disclosure also provides methods of treating a subject having primary hypertension or at risk of developing primary hypertension, the methods comprising administering an SLC9A3R2 inhibitor to the subject.

The present disclosure also provides methods of treating a subject having secondary hypertension or at risk of developing secondary hypertension, the methods comprising administering an SLC9A3R2 inhibitor to the subject.

The present disclosure also provides methods of treating a subject having resistant hypertension or at risk of developing resistant hypertension, the methods comprising administering an SLC9A3R2 inhibitor to the subject.

The present disclosure also provides methods of treating a subject having malignant hypertension or at risk of developing malignant hypertension, the methods comprising administering an SLC9A3R2 inhibitor to the subject.

The present disclosure also provides methods of treating a subject having coronary heart disease or at risk of developing coronary heart disease, the methods comprising administering an SLC9A3R2 inhibitor to the subject.

The present disclosure also provides methods of treating a subject having atrial fibrillation or at risk of developing atrial fibrillation, the methods comprising administering an SLC9A3R2 inhibitor to the subject.

The present disclosure also provides methods of treating a subject with a therapeutic agent that treats or prevents hypertension, coronary heart disease, and/or atrial fibrillation, wherein the subject has hypertension, coronary heart disease, and/or atrial fibrillation or is at risk for developing hypertension, coronary heart disease, and/or atrial fibrillation, the methods comprising: determining whether the subject has an SLC9A3R2 missense variant nucleic acid molecule encoding an SLC9A3R2 predicted loss-of-function polypeptide by: obtaining or having obtained a biological sample from the subject; and performing or having performed a sequence analysis on the biological sample to determine if the subject has a genotype comprising the SLC9A3R2 missense variant nucleic acid molecule encoding the SLC9A3R2 predicted loss-of-function polypeptide; and administering or continuing to administer the therapeutic agent that treats or prevents hypertension, coronary heart disease, and/or atrial fibrillation in a standard dosage amount to a subject that is SLC9A3R2 reference, and/or administering an SLC9A3R2 inhibitor to the subject; and administering or continuing to administer the therapeutic agent that treats or prevents hypertension, coronary heart disease, and/or atrial fibrillation in an amount that is the same as or less than a standard dosage amount to a subject that is heterozygous for the SLC9A3R2 missense variant nucleic acid molecule, and/or administering an SLC9A3R2 inhibitor to the subject; wherein the presence of a genotype having the SLC9A3R2 missense variant nucleic acid molecule encoding the SLC9A3R2 predicted loss-of-function polypeptide indicates the subject has a decreased risk of developing hypertension, coronary heart disease, and/or atrial fibrillation.

The present disclosure also provides methods of identifying a subject having an increased risk of developing hypertension, coronary heart disease, and/or atrial fibrillation, the methods comprising: determining or having determined the presence or absence of an SLC9A3R2 missense variant nucleic acid molecule encoding an SLC9A3R2 predicted loss-of-function polypeptide in a biological sample obtained from the subject; wherein: when the subject is SLC9A3R2 reference, then the subject has an increased risk of developing hypertension, coronary heart disease, and/or atrial fibrillation; and when the subject is heterozygous or homozygous for an SLC9A3R2 missense variant nucleic acid molecule encoding the SLC9A3R2 predicted loss-of-function polypeptide, then the subject has a decreased risk of developing hypertension, coronary heart disease, and/or atrial fibrillation.

The present disclosure also provides therapeutic agents that treat or prevent hypertension, coronary heart disease, and/or atrial fibrillation for use in the treatment or prevention of hypertension, coronary heart disease, and/or atrial fibrillation in a subject identified as having: i) a genomic nucleic acid molecule encoding an SLC9A3R2 predicted loss-of-function polypeptide, or the complement thereof, wherein the genomic nucleic acid molecule has a nucleotide sequence comprising: i) a thymine at a position corresponding to position 9,519 according to SEQ ID NO:2, or the complement thereof; ii) an mRNA molecule encoding an SLC9A3R2 predicted loss-of-function polypeptide, or the complement thereof, wherein the mRNA molecule has a nucleotide sequence comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; or iii) a cDNA molecule encoding an SLC9A3R2 predicted loss-of-function polypeptide, or the complement thereof, wherein the cDNA molecule has a nucleotide sequence comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

The present disclosure also provides SLC9A3R2 inhibitors for use in the treatment or prevention of hypertension, coronary heart disease, and/or atrial fibrillation in a subject that: a) is reference for an SLC9A3R2 genomic nucleic acid molecule, an SLC9A3R2 mRNA molecule, or an SLC9A3R2 cDNA molecule; or b) is heterozygous for: i) a genomic nucleic acid molecule encoding an SLC9A3R2 predicted loss-of-function polypeptide, or the complement thereof, wherein the genomic nucleic acid molecule has a nucleotide sequence comprising: a thymine at a position corresponding to position 9,519 according to SEQ ID NO:2, or the complement thereof; ii) an mRNA molecule encoding an SLC9A3R2 predicted loss-of-function polypeptide, or the complement thereof, wherein the mRNA molecule has a nucleotide sequence comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; or iii) a cDNA molecule encoding an SLC9A3R2 predicted loss-of-function polypeptide, or the complement thereof, wherein the cDNA molecule has a nucleotide sequence comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or other tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A burden of rare putative loss-of-function (LOF) and deleterious missense variants in the SLC9A3R2 gene associated with a decreased risk of developing hypertension in humans has been identified in accordance with the present disclosure. For example, a genetic alteration that changes the cytosine at position 9,519 in the SLC9A3R2 reference genomic nucleic acid molecule (see, SEQ ID NO:1) to a thymine, has been observed to indicate that the subject having such an alteration may have a decreased risk of developing hypertension. It is believed that no variants of the SLC9A3R2 gene or protein have any known association with hypertension. Altogether, the genetic analyses described herein surprisingly indicate that the SLC9A3R2 gene and, in particular, pLOFs and deleterious missense variants in the SLC9A3R2 gene, associates with a decreased risk of developing hypertension. Therefore, subjects that are SLC9A3R2 reference that have an increased risk of developing hypertension, such as primary hypertension, secondary hypertension, resistant hypertension, or malignant hypertension, coronary heart disease, and/or atrial fibrillation, may be treated such that the hypertension, coronary heart disease, and/or atrial fibrillation is prevented, the symptoms thereof are reduced, and/or development of symptoms is repressed. Accordingly, the present disclosure provides methods of leveraging the identification of such variants in subjects to identify or stratify risk in such subjects of developing hypertension, such as primary hypertension, secondary hypertension, resistant hypertension, or malignant hypertension, coronary heart disease, and/or atrial fibrillation, or to diagnose subjects as having an increased risk of developing hypertension, such as primary hypertension, secondary hypertension, resistant hypertension, or malignant hypertension, coronary heart disease, and/or atrial fibrillation, such that subjects at risk or subjects with active disease may be treated accordingly.

It has been further observed in accordance with the present disclosure that an SLC9A3R2 missense variant nucleic acid molecule encoding an SLC9A3R2 predicted loss-of-function polypeptide (whether these variations are homozygous or heterozygous in a particular subject) associate with a decreased risk of developing hypertension. Moreover, the identification by the present disclosure of the association between additional variants and gene burden masks indicates that SLC9A3R2 itself (rather than linkage disequilibrium with variants in another gene) is responsible for a protective effect in hypertension.

For purposes of the present disclosure, any particular subject can be categorized as having one of three SLC9A3R2 genotypes: i) SLC9A3R2 reference; ii) heterozygous for an SLC9A3R2 missense variant nucleic acid molecule encoding an SLC9A3R2 predicted loss-of-function polypeptide; or iii) homozygous for an SLC9A3R2 missense variant nucleic acid molecule encoding an SLC9A3R2 predicted loss-of-function polypeptide. A subject is SLC9A3R2 reference when the subject does not have a copy of an SLC9A3R2 missense variant nucleic acid molecule encoding an SLC9A3R2 predicted loss-of-function polypeptide. A subject is heterozygous for an SLC9A3R2 missense variant nucleic acid molecule encoding an SLC9A3R2 predicted loss-of-function polypeptide when the subject has a single copy of an SLC9A3R2 missense variant nucleic acid molecule. As used herein, an SLC9A3R2 missense variant nucleic acid molecule is any SLC9A3R2 nucleic acid molecule (such as, a genomic nucleic acid molecule, an mRNA molecule, or a cDNA molecule) encoding an SLC9A3R2 polypeptide having a partial loss-of-function, a complete loss-of-function, a predicted partial loss-of-function, or a predicted complete loss-of-function. A subject who has an SLC9A3R2 missense variant nucleic acid molecule encoding an SLC9A3R2 predicted loss-of-function polypeptide having a partial loss-of-function (or predicted partial loss-of-function) is hypomorphic for SLC9A3R2. The SLC9A3R2 missense variant nucleic acid molecule encoding an SLC9A3R2 predicted loss-of-function polypeptide can be any nucleic acid molecule encoding an SLC9A3R2 Arg171Trp-Long, Arg171Trp-Short, Arg65Trp, Arg58Trp, Arg60Trp-Short, Arg60Trp-Long, or Arg170Trp. In some embodiments, the SLC9A3R2 missense variant nucleic acid molecule encodes an SLC9A3R2 Arg171Trp-Long or Arg171Trp-Short. A subject is homozygous for an SLC9A3R2 missense variant nucleic acid molecule encoding an SLC9A3R2 predicted loss-of-function polypeptide when the subject has two copies of an SLC9A3R2 missense variant nucleic acid molecule encoding an SLC9A3R2 predicted loss-of-function polypeptide.

For subjects that are genotyped or determined to be SLC9A3R2 reference, such subjects have an increased risk of developing hypertension, such as primary hypertension, secondary hypertension, resistant hypertension, or malignant hypertension, coronary heart disease, and/or atrial fibrillation. For subjects that are genotyped or determined to be either SLC9A3R2 reference or heterozygous for an SLC9A3R2 missense variant nucleic acid molecule encoding an SLC9A3R2 predicted loss-of-function polypeptide, such subjects can be treated with an SLC9A3R2 inhibitor.

In any of the embodiments described throughout the present disclosure, the SLC9A3R2 missense variant nucleic acid molecule can be any SLC9A3R2 nucleic acid molecule (such as, for example, genomic nucleic acid molecule, mRNA molecule, or cDNA molecule) encoding an SLC9A3R2 polypeptide having a partial loss-of-function, a complete loss-of-function, a predicted partial loss-of-function, or a predicted complete loss-of-function. For example, the SLC9A3R2 missense variant nucleic acid molecule can be any nucleic acid molecule encoding SLC9A3R2 Arg171Trp-Long, Arg171Trp-Short, Arg65Trp, Arg58Trp, Arg60Trp-Short, Arg60Trp-Long, or Arg170Trp. In some embodiments, the SLC9A3R2 missense variant nucleic acid molecule encodes SLC9A3R2 Arg171Trp-Long or Arg171Trp-Short.

In any of the embodiments described throughout the present disclosure, the SLC9A3R2 predicted loss-of-function polypeptide can be any SLC9A3R2 polypeptide having a partial loss-of-function, a complete loss-of-function, a predicted partial loss-of-function, or a predicted complete loss-of-function. In any of the embodiments described throughout the present disclosure, the SLC9A3R2 predicted loss-of-function polypeptide can be any of the SLC9A3R2 polypeptides described herein including, for example, SLC9A3R2 Arg171Trp-Long, Arg171Trp-Short, Arg65Trp, Arg58Trp, Arg60Trp-Short, Arg60Trp-Long, or Arg170Trp. In some embodiments, the SLC9A3R2 predicted loss-of-function polypeptide is SLC9A3R2 Arg171Trp-Long or Arg171Trp-Short.

Any one or more (i.e., any combination) of the SLC9A3R2 missense variant nucleic acid molecules encoding an SLC9A3R2 predicted loss-of-function polypeptide can be used within any of the methods described herein to determine whether a subject has an increased risk of developing hypertension, coronary heart disease, and/or atrial fibrillation. The combinations of particular variants can form a mask used for statistical analysis of the particular correlation of SLC9A3R2 and decreased risk of developing hypertension, coronary heart disease, and/or atrial fibrillation.

In any of the embodiments described throughout the present disclosure, the hypertension is primary hypertension, secondary hypertension, resistant hypertension, or malignant hypertension. In any of the embodiments described throughout the present disclosure, the hypertension is primary hypertension. In any of the embodiments described throughout the present disclosure, the hypertension is secondary hypertension. In any of the embodiments described throughout the present disclosure, the hypertension is resistant hypertension. In any of the embodiments described throughout the present disclosure, the hypertension is malignant hypertension.

Symptoms of hypertension include, but are not limited to, increased blood pressure, headaches, shortness of breath, nosebleeds, flushing, dizziness, chest pain, visual changes, and/or blood in the urine.

The present disclosure provides methods of treating a subject having hypertension or at risk of developing hypertension, the methods comprising administering an SLC9A3R2 inhibitor to the subject.

The present disclosure also provides methods of treating a subject having primary hypertension or at risk of developing primary hypertension, the methods comprising administering an SLC9A3R2 inhibitor to the subject.

The present disclosure also provides methods of treating a subject having secondary hypertension or at risk of developing secondary hypertension, the methods comprising administering an SLC9A3R2 inhibitor to the subject.

The present disclosure also provides methods of treating a subject having resistant hypertension or at risk of developing resistant hypertension, the methods comprising administering an SLC9A3R2 inhibitor to the subject.

The present disclosure also provides methods of treating a subject having malignant hypertension or at risk of developing malignant hypertension, the methods comprising administering an SLC9A3R2 inhibitor to the subject.

The present disclosure also provides methods of treating a subject having coronary heart disease or at risk of developing coronary heart disease, the methods comprising administering an SLC9A3R2 inhibitor to the subject.

The present disclosure also provides methods of treating a subject having atrial fibrillation or at risk of developing atrial fibrillation, the methods comprising administering an SLC9A3R2 inhibitor to the subject.

In some embodiments, the SLC9A3R2 inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of an SLC9A3R2 nucleic acid molecule, such as an mRNA molecule. In some embodiments, the inhibitory nucleic acid molecule hybridizes to a sequence within an SLC9A3R2 genomic nucleic acid molecule or mRNA molecule and decreases expression of the SLC9A3R2 polypeptide in a cell in the subject. In some embodiments, the SLC9A3R2 inhibitor comprises an antisense RNA that hybridizes to an SLC9A3R2 genomic nucleic acid molecule or mRNA molecule and decreases expression of the SLC9A3R2 polypeptide in a cell in the subject. In some embodiments, the SLC9A3R2 inhibitor comprises an siRNA that hybridizes to an SLC9A3R2 genomic nucleic acid molecule or mRNA molecule and decreases expression of the SLC9A3R2 polypeptide in a cell in the subject. In some embodiments, the SLC9A3R2 inhibitor comprises an shRNA that hybridizes to an SLC9A3R2 genomic nucleic acid molecule or mRNA molecule and decreases expression of the SLC9A3R2 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense:
mN*mN*/i2FN/mN/2FN/mN/i2FN/mN/2FN/mN/i2FN/mN/2FN/
mN/i2FN/mN/i2FN/*mN*/32FN/
Antisense:
/52FN/*/i2FN/*mN/i2FN/mN/i2FN/mN/i2FN/mN/i2FN/
mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the SLC9A3R2 inhibitor comprises a nuclease agent that induces one or more nicks or double-strand breaks at a recognition sequence(s) or a DNA-binding protein that binds to a recognition sequence within an SLC9A3R2 genomic nucleic acid molecule. The recognition sequence can be located within a coding region of the SLC9A3R2 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the SLC9A3R2 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30 to about 36 bp for a zinc finger protein or ZFN pair, about 15 to about 18 bp for each ZFN, about 36 bp for a TALE protein or TALEN, and about 20 bp for a CRISPR/Cas guide RNA.

In some embodiments, CRISPR/Cas systems can be used to modify an SLC9A3R2 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of SLC9A3R2 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an SLC9A3R2 genomic nucleic acid molecule or it can be a nickase that creates a single-strand break in an SLC9A3R2 genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of an SLC9A3R2 genomic nucleic acid molecules can be generated by contacting a cell with a Cas protein and one or more gRNAs that hybridize to one or more gRNA recognition sequences within a target genomic locus in the SLC9A3R2 genomic nucleic acid molecule. For example, a gRNA recognition sequence can be located within a region of SEQ ID NO:1. The gRNA recognition sequence can also include or be proximate to a position corresponding to: position 9,519 according to SEQ ID NO:1. For example, the gRNA recognition sequence can be located about 1000, about 500, about 400, about 300, about 200, about 100, about 50, about 45, about 40, about 35, about 30, about 25, about 20, about 15, about 10, or about 5 nucleotides from a position corresponding to: position 9,519 according to SEQ ID NO:1. The gRNA recognition sequence can include or be proximate to the start codon or the stop codon of an SLC9A3R2 genomic nucleic acid molecule. For example, the gRNA recognition sequence can be located about 10, about 20, about 30, about 40, about 50, about 100, about 200, about 300, about 400, about 500, or about 1,000 nucleotides from the start codon or the stop codon.

The gRNA recognition sequences within a target genomic locus in an SLC9A3R2 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes a PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an SLC9A3R2 genomic nucleic acid molecule. An exemplary gRNA is a gRNA effective to direct a Cas enzyme to bind to or cleave an SLC9A3R2 genomic nucleic acid molecule, wherein the gRNA comprises a DNA-targeting segment that hybridizes to a gRNA recognition sequence within the SLC9A3R2 genomic nucleic acid molecule that includes or is proximate to a position corresponding to: position 9,519 according to SEQ ID NO:1. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from a position corresponding to: position 9,519 according to SEQ ID NO:1. Other exemplary gRNAs comprise a DNA-targeting segment that hybridizes to a gRNA recognition sequence present within an SLC9A3R2 genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from the start codon or located about 5, about 10, about 15, about 20, about 25, about 30, about 35, about 40, about 45, about 50, about 100, about 200, about 300, about 400, about 500, or about 1,000 nucleotides from the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs comprise 20 nucleotides.

Examples of suitable gRNA recognition sequences located within the SLC9A3R2 reference gene are set forth in Table 1 as SEQ ID NOs:93-112.

TABLE 1
Guide RNA Recognition Sequences
Near SLC9A3R2 Variation

| Strand | gRNA Recognition Sequence | SEQ ID NO: |
|---|---|---|
| − | AGGTTGAACCCATAGCCCTG | 93 |
| + | CACCTGCGAAAGGGACCTCA | 94 |
| + | GTCAACGGCGTCAACGTGGA | 95 |
| + | GGATGTCAGTGGGCCCCTGA | 96 |
| − | TGACGCCGTTGACCTCGACC | 97 |
| + | ACCTGCATAGTGACAAGTCC | 98 |
| + | TGGTGGCCAGCATCAAGGCA | 99 |
| − | GCCGGGACTTGTCACTATGC | 100 |
| + | GTGAACGGGCAGAATGTGGA | 101 |
| + | TTCCACCTGCACGGCGAGAA | 102 |
| + | AAGCCAGACTGGGCACACAC | 103 |
| + | CGGCCAGTACATCCGCTCTG | 104 |
| + | TACATCCGCTCTGTGGACCC | 105 |
| + | CGAGGTCAACGGCGTCAACG | 106 |
| − | ACAGAGCGGATGTACTGGCC | 107 |
| − | CGCGCCGGATGAACTGCCCG | 108 |
| + | GCGGCAGCTGACCTGTACCG | 109 |
| − | GGGCCGGTACCTCAATGAGC | 110 |
| + | AGGCTGTGGAGGGGCAGACT | 111 |
| − | GCAGCGCGGCGGCCTCGGCG | 112 |

The Cas protein and the gRNA form a complex, and the Cas protein cleaves the target SLC9A3R2 genomic nucleic acid molecule. The Cas protein can cleave the nucleic acid molecule at a site within or outside of the nucleic acid sequence present in the target SLC9A3R2 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the SLC9A3R2 genomic nucleic acid molecule to which a DNA-targeting segment of a gRNA will bind.

Such methods can result, for example, in an SLC9A3R2 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the SLC9A3R2 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the SLC9A3R2 inhibitor comprises a small molecule.

In some embodiments, the methods of treatment further comprise detecting the presence or absence of an SLC9A3R2 missense variant nucleic acid molecule encoding an SLC9A3R2 predicted loss-of-function polypeptide in a biological sample obtained from the subject. As used throughout the present disclosure, “an SLC9A3R2 missense variant nucleic acid molecule” is any SLC9A3R2 nucleic acid molecule (such as, for example, genomic nucleic acid molecule, mRNA molecule, or cDNA molecule) encoding an SLC9A3R2 polypeptide having a partial loss-of-function, a complete loss-of-function, a predicted partial loss-of-function, or a predicted complete loss-of-function.

The present disclosure also provides methods of treating a subject with a therapeutic agent that treats or prevents hypertension, coronary heart disease, and/or atrial fibrillation. In some embodiments, the subject has hypertension. In some embodiments, the subject is at risk of developing hypertension. In some embodiments, the subject has coronary heart disease. In some embodiments, the subject is at risk of developing coronary heart disease. In some embodiments, the subject has atrial fibrillation. In some embodiments, the subject is at risk of developing atrial fibrillation. In some embodiments, the methods comprise determining whether the subject has an SLC9A3R2 missense variant nucleic acid molecule encoding an SLC9A3R2 predicted loss-of-function polypeptide by obtaining or having obtained a biological sample obtained from the subject, and performing or having performed a sequence analysis on the biological sample to determine if the subject has a genotype comprising the SLC9A3R2 missense variant nucleic acid molecule. When the subject is SLC9A3R2 reference, the therapeutic agent that treats or prevents hypertension, coronary heart disease, and/or atrial fibrillation is administered or continued to be administered to the subject in a standard dosage amount, and/or an SLC9A3R2 inhibitor is administered to the subject. When the subject is heterozygous for an SLC9A3R2 missense variant, the therapeutic agent that treats or prevents hypertension, coronary heart disease, and/or atrial fibrillation is administered or continued to be administered to the subject in an amount that is the same as or less than a standard dosage amount, and/or an SLC9A3R2 inhibitor is administered to the subject. The presence of a genotype having the SLC9A3R2 missense variant nucleic acid molecule encoding the SLC9A3R2 predicted loss-of-function polypeptide indicates the subject has a decreased risk of developing hypertension, coronary heart disease, and/or atrial fibrillation. In some embodiments, the subject is SLC9A3R2 reference. In some embodiments, the subject is heterozygous for the SLC9A3R2 missense variant nucleic acid molecule encoding an SLC9A3R2 predicted loss-of-function polypeptide.

For subjects that are genotyped or determined to be either SLC9A3R2 reference or heterozygous for the SLC9A3R2 missense variant nucleic acid molecule encoding an SLC9A3R2 predicted loss-of-function polypeptide, such subjects can be treated with an SLC9A3R2 inhibitor, as described herein.

Detecting the presence or absence of an SLC9A3R2 missense variant nucleic acid molecule encoding an SLC9A3R2 predicted loss-of-function polypeptide in a biological sample obtained from a subject and/or determining whether a subject has an SLC9A3R2 missense variant nucleic acid molecule encoding an SLC9A3R2 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the SLC9A3R2 missense variant nucleic acid molecule encoding an SLC9A3R2 predicted loss-of-function polypeptide can be present within a cell obtained from the subject.

In some embodiments, when the subject is SLC9A3R2 reference, the subject is also administered a therapeutic agent that treats or prevents hypertension, coronary heart disease, and/or atrial fibrillation in a standard dosage amount. In some embodiments, when the subject is heterozygous for an SLC9A3R2 missense variant nucleic acid molecule encoding an SLC9A3R2 predicted loss-of-function polypeptide, the subject is administered a therapeutic agent that treats or prevents hypertension, coronary heart disease, and/or atrial fibrillation in a dosage amount that is the same as or less than a standard dosage amount.

In some embodiments, the treatment methods further comprise detecting the presence or absence of an SLC9A3R2 predicted loss-of-function polypeptide in a biological sample obtained from the subject. In some embodiments, when the subject does not have an SLC9A3R2 predicted loss-of-function polypeptide, the subject is administered a therapeutic agent that treats or prevents hypertension, coronary heart disease, and/or atrial fibrillation in a standard dosage amount. In some embodiments, when the subject has an SLC9A3R2 predicted loss-of-function polypeptide, the subject is administered a therapeutic agent that treats or prevents hypertension, coronary heart disease, and/or atrial fibrillation in a dosage amount that is the same as or less than a standard dosage amount.

The present disclosure also provides methods of treating a subject with a therapeutic agent that treats or prevents hypertension, coronary heart disease, and/or atrial fibrillation. In some embodiments, the subject has hypertension. In some embodiments, the subject is at risk of developing hypertension. In some embodiments, the subject has coronary heart disease. In some embodiments, the subject is at risk of developing coronary heart disease. In some embodiments, the subject has atrial fibrillation. In some embodiments, the subject is at risk of developing atrial fibrillation. In some embodiments, the methods comprise determining whether the subject has an SLC9A3R2 predicted loss-of-function polypeptide by obtaining or having obtained a biological sample from the subject, and performing or having performed an assay on the biological sample to determine if the subject has an SLC9A3R2 predicted loss-of-function polypeptide. When the subject does not have an SLC9A3R2 predicted loss-of-function polypeptide, the therapeutic agent that treats or prevents hypertension, coronary heart disease, and/or atrial fibrillation is administered or continued to be administered to the subject in a standard dosage amount, and/or an SLC9A3R2 inhibitor is administered to the subject. When the subject has an SLC9A3R2 predicted loss-of-function polypeptide, the therapeutic agent that treats or prevents hypertension, coronary heart disease, and/or atrial fibrillation is administered or continued to be administered to the subject in an amount that is the same as or less than a standard dosage amount, and/or an SLC9A3R2 inhibitor is administered to the subject. The presence of an SLC9A3R2 predicted loss-of-function polypeptide indicates the subject has a decreased risk of developing hypertension, coronary heart disease, and/or atrial fibrillation. In some embodiments, the subject has an SLC9A3R2 predicted loss-of-function polypeptide. In some embodiments, the subject does not have an SLC9A3R2 predicted loss-of-function polypeptide.

Detecting the presence or absence of an SLC9A3R2 predicted loss-of-function polypeptide in a biological sample obtained from a subject and/or determining whether a subject has an SLC9A3R2 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the SLC9A3R2 predicted loss-of-function polypeptide can be present within a cell obtained from the subject.

Examples of therapeutic agents that treat or prevent hypertension include, but are not limited to: thiazide diuretics (such as, chlorthalidone, chlorothiazide, hydrochlorothiazide, indapamide, or metolazone); potassium-sparing diuretics (such as, amiloride, spironolactone, or triamterene); loop diuretics (such as, bumetanide, furosemide, or torsemide); beta blockers (such as, acebutolol, atenolol, betaxolol, bisoprolol, bisoprolol/hydrochlorothiazide, metoprolol tartrate, metoprolol succinate, nadolol, pindolol, propranolol, solotol, or timolol); angiotensin converting enzyme (ACE) inhibitors (such as, benazepril, captopril, enalapril, fosinopril, lisinopril, moexipril, perindopril, quinapril, ramipril, or trandolapril); angiotensin II receptor blockers (ARBs) (such as, candesartan, eprosartan, irbesartan, losartan, telmisartan, or valsartan); calcium channel blockers (such as, amlodipine, diltiazem, felodipine, isradipine, nicardipine, nifedipine, nisoldipine, or verapamil); alpha-blockers (such as, doxazosin, prazosin, or terazosin); alpha-beta-blockers (such as carvedilol or labetalol); central agonists (such as, methyldopa, clonidine, or guanfacine); vasodilators (such as, hydralazine or minoxidil); aldosterone receptor antagonists (such as, eplerenone or spironolactone), and renin inhibitors (such as aliskiren).

In some embodiments, the therapeutic agent that treats or prevents hypertension is a thiazide diuretic, a potassium-sparing diuretic, a loop diuretic, a beta blocker, an ACE inhibitor, an ARB, a calcium channel blocker, an alpha-blocker, an alpha-beta-blocker, a central agonist, a vasodilator, an aldosterone receptor antagonist, or a renin inhibitor. In some embodiments, the thiazide diuretic is chlorthalidone, chlorothiazide, hydrochlorothiazide, indapamide, or metolazone. In some embodiments, the potassium-sparing diuretic is amiloride, spironolactone, or triamterene. In some embodiments, the loop diuretic is bumetanide, furosemide, or torsemide. In some embodiments, the beta blocker is acebutolol, atenolol, betaxolol, bisoprolol, bisoprolol/hydrochlorothiazide, metoprolol tartrate, metoprolol succinate, nadolol, pindolol, propranolol, solotol, or timolol). In some embodiments, the ACE inhibitor is benazepril, captopril, enalapril, fosinopril, lisinopril, moexipril, perindopril, quinapril, ramipril, or trandolapril. In some embodiments, the ARB is candesartan, eprosartan, irbesartan, losartan, telmisartan, or valsartan. In some embodiments, the calcium channel blocker is amlodipine, diltiazem, felodipine, isradipine, nicardipine, nifedipine, nisoldipine, or verapamil. In some embodiments, the alpha-blocker is doxazosin, prazosin, or terazosin. In some embodiments, the alpha-beta-blocker is carvedilol or labetalol. In some embodiments, the central agonist is methyldopa, clonidine, or guanfacine). In some embodiments, the vasodilator is hydralazine or minoxidil. In some embodiments, the aldosterone receptor antagonist is eplerenone or spironolactone. In some embodiments, the renin inhibitor is aliskiren.

In some embodiments, the dose of the therapeutic agents that treat or prevent hypertension, coronary heart disease, and/or atrial fibrillation can be reduced by about 10%, by about 20%, by about 30%, by about 40%, by about 50%, by about 60%, by about 70%, by about 80%, or by about 90% for subjects that are heterozygous for an SLC9A3R2 missense variant nucleic acid molecule encoding an SLC9A3R2 predicted loss-of-function polypeptide (i.e., less than the standard dosage amount) compared to subjects that are SLC9A3R2 reference (who may receive a standard dosage amount). In some embodiments, the dose of the therapeutic agents that treat or prevent hypertension, coronary heart disease, and/or atrial fibrillation can be reduced by about 10%, by about 20%, by about 30%, by about 40%, or by about 50%. In addition, the dose of therapeutic agents that treat or prevent hypertension, coronary heart disease, and/or atrial fibrillation in subjects that are heterozygous for an SLC9A3R2 missense variant nucleic acid molecule encoding an SLC9A3R2 predicted loss-of-function polypeptide can be administered less frequently compared to subjects that are SLC9A3R2 reference.

Administration of the therapeutic agents that treat or prevent hypertension, coronary heart disease, and/or atrial fibrillation and/or SLC9A3R2 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or prevent hypertension, coronary heart disease, and/or atrial fibrillation and/or SLC9A3R2 inhibitors can be administered sequentially or at the same time. In addition, the therapeutic agents that treat or prevent hypertension, coronary heart disease, and/or atrial fibrillation and/or SLC9A3R2 inhibitors can be administered in separate compositions or can be administered together in the same composition.

Administration of the therapeutic agents that treat or prevent hypertension, coronary heart disease, and/or atrial fibrillation and/or SLC9A3R2 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in hypertension, a decrease/reduction in the severity of hypertension (such as, for example, a reduction or inhibition of development of hypertension), a decrease/reduction in symptoms and hypertension-related effects, delaying the onset of symptoms and hypertension-related effects, reducing the severity of symptoms of hypertension-related effects, reducing the severity of an acute episode, reducing the number of symptoms and hypertension-related effects, reducing the latency of symptoms and hypertension-related effects, an amelioration of symptoms and hypertension-related effects, reducing secondary symptoms, reducing secondary infections, preventing relapse to hypertension, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of hypertension development/progression (such as, for example, a complete or partial avoidance/inhibition or a delay), and an increased survival time of the affected host animal, following administration of a therapeutic protocol. Treatment of hypertension encompasses the treatment of subjects already diagnosed as having any form of hypertension at any clinical stage or manifestation, the delay of the onset or evolution or aggravation or deterioration of the symptoms or signs of hypertension, and/or preventing and/or reducing the severity of hypertension.

The present disclosure also provides methods of identifying a subject having an increased risk of developing hypertension, coronary heart disease, and/or atrial fibrillation. In some embodiments, the methods comprise determining or having determined the presence or absence of an SLC9A3R2 missense variant nucleic acid molecule (such as a genomic nucleic acid molecule, mRNA molecule, and/or cDNA molecule) encoding an SLC9A3R2 predicted loss-of-function polypeptide in a biological sample obtained from the subject. When the subject lacks an SLC9A3R2 missense variant nucleic acid molecule encoding an SLC9A3R2 predicted loss-of-function polypeptide (i.e., the subject is genotypically categorized as SLC9A3R2 reference), then the subject has an increased risk of developing hypertension, coronary heart disease, and/or atrial fibrillation. When the subject has an SLC9A3R2 missense variant nucleic acid molecule encoding an SLC9A3R2 predicted loss-of-function polypeptide (i.e., the subject is heterozygous or homozygous for an SLC9A3R2 missense variant nucleic acid molecule), then the subject has a decreased risk of developing hypertension, coronary heart disease, and/or atrial fibrillation compared to a subject that is SLC9A3R2 reference.

Having a single copy of an SLC9A3R2 missense variant nucleic acid molecule encoding an SLC9A3R2 predicted loss-of-function polypeptide is more protective of a subject from developing hypertension, coronary heart disease, and/or atrial fibrillation than having no copies of an SLC9A3R2 missense variant nucleic acid molecule encoding an SLC9A3R2 predicted loss-of-function polypeptide. Without intending to be limited to any particular theory or mechanism of action, it is believed that a single copy of an SLC9A3R2 missense variant nucleic acid molecule (i.e., heterozygous for an SLC9A3R2 missense variant nucleic acid molecule) is protective of a subject from developing hypertension, coronary heart disease, and/or atrial fibrillation, and it is also believed that having two copies of an SLC9A3R2 missense variant nucleic acid molecule encoding an SLC9A3R2 predicted loss-of-function polypeptide (i.e., homozygous for an SLC9A3R2 missense variant nucleic acid molecule) may be more protective of a subject from developing hypertension, coronary heart disease, and/or atrial fibrillation, relative to a subject with a single copy. Thus, in some embodiments, a single copy of an SLC9A3R2 missense variant nucleic acid molecule encoding an SLC9A3R2 predicted loss-of-function polypeptide may not be completely protective, but instead, may be partially or incompletely protective of a subject from developing hypertension, coronary heart disease, and/or atrial fibrillation. While not desiring to be bound by any particular theory, there may be additional factors or molecules involved in the development of hypertension, coronary heart disease, and/or atrial fibrillation that are still present in a subject having a single copy of an SLC9A3R2 missense variant nucleic acid molecule encoding an SLC9A3R2 predicted loss-of-function polypeptide, thus resulting in less than complete protection from the development of hypertension, coronary heart disease, and/or atrial fibrillation.

Detecting the presence or absence of an SLC9A3R2 missense variant nucleic acid molecule encoding an SLC9A3R2 predicted loss-of-function polypeptide in a biological sample obtained from a subject and/or determining whether a subject has an SLC9A3R2 missense variant nucleic acid molecule encoding an SLC9A3R2 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the SLC9A3R2 missense variant nucleic acid molecule encoding an SLC9A3R2 predicted loss-of-function polypeptide can be present within a cell obtained from the subject.

In some embodiments, when a subject is identified as having an increased risk of developing hypertension, coronary heart disease, and/or atrial fibrillation, the subject is further treated with a therapeutic agent that treats or prevents hypertension, coronary heart disease, and/or atrial fibrillation and/or an SLC9A3R2 inhibitor, as described herein. For example, when the subject is SLC9A3R2 reference, and therefore has an increased risk for developing hypertension, coronary heart disease, and/or atrial fibrillation, the subject is administered an SLC9A3R2 inhibitor. In some embodiments, such a subject is also administered a therapeutic agent that treats or prevents hypertension, coronary heart disease, and/or atrial fibrillation. In some embodiments, when the subject is heterozygous for an SLC9A3R2 missense variant nucleic acid molecule encoding an SLC9A3R2 predicted loss-of-function polypeptide, the subject is administered the therapeutic agent that treats or prevents hypertension, coronary heart disease, and/or atrial fibrillation in a dosage amount that is the same as or less than a standard dosage amount, and/or is administered an SLC9A3R2 inhibitor. In some embodiments, the subject is SLC9A3R2 reference. In some embodiments, the subject is heterozygous for an SLC9A3R2 missense variant nucleic acid molecule encoding an SLC9A3R2 predicted loss-of-function polypeptide.

In some embodiments, any of the methods described herein can further comprise determining the subject's aggregate burden of having an SLC9A3R2 missense variant nucleic acid molecule encoding an SLC9A3R2 predicted loss-of-function polypeptide, and/or an SLC9A3R2 predicted loss-of-function variant polypeptide associated with a decreased risk of developing hypertension, coronary heart disease, and/or atrial fibrillation. The aggregate burden is the sum of all variants in the SLC9A3R2 gene, which can be carried out in an association analysis with hypertension, coronary heart disease, and/or atrial fibrillation. In some embodiments, the subject is homozygous for one or more SLC9A3R2 missense variant nucleic acid molecules encoding an SLC9A3R2 predicted loss-of-function polypeptide associated with a decreased risk of developing hypertension, coronary heart disease, and/or atrial fibrillation. In some embodiments, the subject is heterozygous for one or more SLC9A3R2 missense variant nucleic acid molecules encoding an SLC9A3R2 predicted loss-of-function polypeptide associated with a decreased risk of developing hypertension, coronary heart disease, and/or atrial fibrillation. The result of the association analysis suggests that SLC9A3R2 missense variant nucleic acid molecules encoding an SLC9A3R2 predicted loss-of-function polypeptide are associated with decreased risk of developing hypertension, coronary heart disease, and/or atrial fibrillation. When the subject has a lower aggregate burden, the subject is at a higher risk of developing hypertension, coronary heart disease, and/or atrial fibrillation and the subject is administered or continued to be administered the therapeutic agent that treats or prevents hypertension, coronary heart disease, and/or atrial fibrillation in a standard dosage amount, and/or an SLC9A3R2 inhibitor. When the subject has a greater aggregate burden, the subject is at a lower risk of developing hypertension, coronary heart disease, and/or atrial fibrillation and the subject is administered or continued to be administered the therapeutic agent that treats or prevents hypertension, coronary heart disease, and/or atrial fibrillation in an amount that is the same as or less than the standard dosage amount. The greater the aggregate burden, the lower the risk of developing hypertension, coronary heart disease, and/or atrial fibrillation.

SLC9A3R2 variants that can be used in the aggregate burden analysis include any one or more, or any combination, of the following:

| Variant | rsID | Transcript IDs |
|---|---|---|
| 16:2027006:A:G | | ENST00000424542:ENST00000432365 |
| 16:2027007:T:A | | ENST00000424542:ENST00000432365 |
| 16:2027007: | | ENST00000424542:ENST00000432365 |
| TGGCCGCGCCGGAGCCGCTGC:T | | |
| 16:2027008:G:A | | ENST00000424542:ENST00000432365 |
| 16:2027009:G:A | | ENST00000424542:ENST00000432365 |
| 16:2027010:C:T | | ENST00000424542:ENST00000432365 |
| 16:2027012:G:A | rs1193332097 | ENST00000424542:ENST00000432365 |
| 16:2027016:C:T | | ENST00000424542:ENST00000432365 |
| 16:2027018:G:A | | ENST00000424542:ENST00000432365 |
| 16:2027019:A:G | | ENST00000424542:ENST00000432365 |
| 16:2027021:C:T | | ENST00000424542:ENST00000432365 |
| 16:2027021:C:A | | ENST00000424542:ENST00000432365 |
| 16:2027028:G:C | | ENST00000424542:ENST00000432365 |
| 16:2027028:G:GGCCGCGCCTGT | | ENST00000424542:ENST00000432365 |
| 16:2027031:C:T | | ENST00000424542:ENST00000432365 |
| 16:2027034:G:A | rs1361655747 | ENST00000424542:ENST00000432365 |
| 16:2027036:C:G | | ENST00000424542:ENST00000432365 |
| 16:2027042:C:T | | ENST00000424542:ENST00000432365 |
| 16:2027043:G:T | | ENST00000424542:ENST00000432365 |
| 16:2027047:G:C | | ENST00000424542:ENST00000432365 |
| 16:2027048:G:A | | ENST00000424542:ENST00000432365 |
| 16:2027049:T:G | | ENST00000424542:ENST00000432365 |
| 16:2027051:C:A | rs916953221 | ENST00000424542:ENST00000432365 |
| 16:2027051:C:G | | ENST00000424542:ENST00000432365 |
| 16:2027052:G:A | | ENST00000424542:ENST00000432365 |
| 16:2027054:G:A | | ENST00000424542:ENST00000432365 |
| 16:2027057:G:A | | ENST00000424542:ENST00000432365 |
| 16:2027058:A:T | rs1442639124 | ENST00000424542:ENST00000432365 |
| 16:2027058:A:G | | ENST00000424542:ENST00000432365 |
| 16:2027059:G:T | rs1185404837 | ENST00000424542:ENST00000432365 |
| 16:2027060:C:T | | ENST00000424542:ENST00000432365 |
| 16:2027060:C:G | | ENST00000424542:ENST00000432365 |
| 16:2027061:A:G | rs1218941774 | ENST00000424542:ENST00000432365 |
| 16:2027061:A:C | | ENST00000424542:ENST00000432365 |
| 16:2027066:T:C | | ENST00000424542:ENST00000432365 |
| 16:2027067:A:G | rs949666306 | ENST00000424542:ENST00000432365 |
| 16:2027069:G:A | rs1448390406 | ENST00000424542:ENST00000432365 |
| 16:2027072:T:C | | ENST00000424542:ENST00000432365 |
| 16:2027073:T:G | | ENST00000424542:ENST00000432365 |
| 16:2027075:C:T | rs1165769843 | ENST00000424542:ENST00000432365 |
| 16:2027078:C:G | | ENST00000424542:ENST00000432365 |
| 16:2027079:T:C | | ENST00000424542:ENST00000432365 |
| 16:2027081:C:T | | ENST00000424542:ENST00000432365 |
| 16:2027084:G:C | rs1410211484 | ENST00000424542:ENST00000432365 |
| 16:2027084:G:A | rs1410211484 | ENST00000424542:ENST00000432365 |
| 16:2027087:G:C | | ENST00000424542:ENST00000432365 |
| 16:2027088:A:G | rs979851726 | ENST00000424542:ENST00000432365 |
| 16:2027089:G:C | rs73496087 | ENST00000424542:ENST00000432365 |
| 16:2027092:G:C | | ENST00000424542:ENST00000432365 |
| 16:2027093:G:C | | ENST00000424542:ENST00000432365 |
| 16:2027096:C:T | rs1339321000 | ENST00000424542:ENST00000432365 |
| 16:2027097:G:A | | ENST00000424542:ENST00000432365 |
| 16:2027099:C:G | | ENST00000424542:ENST00000432365 |
| 16:2027099:C:A | | ENST00000424542:ENST00000432365 |
| 16:2027100:G:A | | ENST00000424542:ENST00000432365 |
| 16:2027102:G:A | rs938208273 | ENST00000424542:ENST00000432365 |
| 16:2027103:G:A | | ENST00000424542:ENST00000432365 |
| 16:2027105:C:T | | ENST00000424542:ENST00000432365 |
| 16:2027106:A:G | rs1226724532 | ENST00000424542:ENST00000432365 |
| 16:2027108:T:G | | ENST00000424542:ENST00000432365 |
| 16:2027110:C:G | | ENST00000424542:ENST00000432365 |
| 16:2027114:C:T | | ENST00000424542:ENST00000432365 |
| 16:2027115:G:A | | ENST00000424542:ENST00000432365 |
| 16:2027115:G:T | rs1180388702 | ENST00000424542:ENST00000432365 |
| 16:2027117:C:T | rs1468684310 | ENST00000424542:ENST00000432365 |
| 16:2027118:G:A | | ENST00000424542:ENST00000432365 |
| 16:2027120:G:A | | ENST00000424542:ENST00000432365 |
| 16:2027121:T:C | | ENST00000424542:ENST00000432365 |
| 16:2027125:A:T | | ENST00000424542:ENST00000432365 |
| 16:2027126:C:G | | ENST00000424542:ENST00000432365 |
| 16:2027127:C:T | | ENST00000424542:ENST00000432365 |
| 16:2027130:G:T | rs745867338 | ENST00000424542:ENST00000432365 |
| 16:2027133:C:T | rs1459864440 | ENST00000424542:ENST00000432365 |
| 16:2027133:C:A | | ENST00000424542:ENST00000432365 |
| 16:2027135:C:T | rs1205449453 | ENST00000424542:ENST00000432365 |
| 16:2027141:G:A | | ENST00000424542:ENST00000432365 |
| 16:2027141:G:T | | ENST00000424542:ENST00000432365 |
| 16:2027144:G:A | rs779649978 | ENST00000424542:ENST00000432365 |
| 16:2027145:C:T | rs749125351 | ENST00000424542:ENST00000432365 |
| 16:2027145:C:A | | ENST00000424542:ENST00000432365 |
| 16:2027148:C:CCGCGCTG | | ENST00000424542:ENST00000432365 |
| 16:2027148:C:T | | ENST00000424542:ENST00000432365 |
| 16:2027148:CCGCGCTGCGCGCTGGG:C | | ENST00000424542:ENST00000432365 |
| 16:2027150:G:A | rs1401663512 | ENST00000424542:ENST00000432365 |
| 16:2027157:G:T | rs774090794 | ENST00000424542:ENST00000432365 |
| 16:2027159:G:T | rs1179064305 | ENST00000424542:ENST00000432365 |
| 16:2027160:C:A | | ENST00000424542:ENST00000432365 |
| 16:2027161:TG:T | rs1407429852 | ENST00000424542:ENST00000432365 |
| 16:2027163:G:A | | ENST00000424542:ENST00000432365 |
| 16:2027165:GA:G | | ENST00000424542:ENST00000432365 |
| 16:2027166:A:G | rs1382164078 | ENST00000424542:ENST00000432365 |
| 16:2027168:C:A | | ENST00000424542:ENST00000432365 |
| 16:2027168:C:T | | ENST00000424542:ENST00000432365 |
| 16:2027169:G:T | | ENST00000424542:ENST00000432365 |
| 16:2027171:C:G | | ENST00000424542:ENST00000432365 |
| 16:2027174:G:C | | ENST00000424542:ENST00000432365 |
| 16:2027177:G:C | | ENST00000424542:ENST00000432365 |
| 16:2027177:G:A | | ENST00000424542:ENST00000432365 |
| 16:2027179:G:T | | ENST00000424542:ENST00000432365 |
| 16:2027180:G:C | | ENST00000424542:ENST00000432365 |
| 16:2027184:A:G | rs945661232 | ENST00000424542:ENST00000432365 |
| 16:2027187:G:A | | ENST00000424542:ENST00000432365 |
| 16:2027189:G:A | | ENST00000424542:ENST00000432365 |
| 16:2027193:A:T | | ENST00000424542:ENST00000432365 |
| 16:2027193:A:C | | ENST00000424542:ENST00000432365 |
| 16:2027196:T:C | | ENST00000424542:ENST00000432365 |
| 16:2027198:G:A | | ENST00000424542:ENST00000432365 |
| 16:2027201:G:A | rs1462925706 | ENST00000424542:ENST00000432365 |
| 16:2027201:G:C | | ENST00000424542:ENST00000432365 |
| 16:2027202:G:A | rs1263561129 | ENST00000424542:ENST00000432365 |
| 16:2027205:A:G | rs998101435 | ENST00000424542:ENST00000432365 |
| 16:2027210:C:G | | ENST00000424542:ENST00000432365 |
| 16:2027213:C:T | | ENST00000424542:ENST00000432365 |
| 16:2027216:C:A | | ENST00000424542:ENST00000432365 |
| 16:2027216:C:T | rs1423488459 | ENST00000424542:ENST00000432365 |
| 16:2027219:G:GT | | ENST00000424542:ENST00000432365 |
| 16:2027220:T:C | | ENST00000424542:ENST00000432365 |
| 16:2029582:G:A | rs1363802911 | ENST00000424542:ENST00000432365 |
| 16:2029586:T:C | rs1304048292 | ENST00000424542:ENST00000432365 |
| 16:2029588:C:G | | ENST00000424542:ENST00000432365 |
| 16:2029588:C:A | | ENST00000424542:ENST00000432365 |
| 16:2029593:G:T | | ENST00000424542:ENST00000432365 |
| 16:2029595:T:A | | ENST00000424542:ENST00000432365 |
| 16:2029595:T:G | | ENST00000424542:ENST00000432365 |
| 16:2029596:C:G | rs752927917 | ENST00000424542:ENST00000432365 |
| 16:2029600:G:A | rs886385658 | ENST00000424542:ENST00000432365 |
| 16:2029601:C:G | | ENST00000424542:ENST00000432365 |
| 16:2029603:G:A | rs1336210715 | ENST00000424542:ENST00000432365 |
| 16:2029604:T:C | rs376442210 | ENST00000424542:ENST00000432365 |
| 16:2029606:G:C | | ENST00000424542:ENST00000432365 |
| 16:2029606:G:A | | ENST00000424542:ENST00000432365 |
| 16:2029611:GCAGA:G | | ENST00000424542:ENST00000432365 |
| 16:2029614:G:T | | ENST00000424542:ENST00000432365 |
| 16:2029615:A:G | | ENST00000424542:ENST00000432365 |
| 16:2029616:C:T | | ENST00000424542:ENST00000432365 |
| 16:2029618:C:T | rs553507988 | ENST00000424542:ENST00000432365 |
| 16:2029618:C:G | | ENST00000424542:ENST00000432365 |
| 16:2029619:G:T | | ENST00000424542:ENST00000432365 |
| 16:2029619:G:A | rs771855196 | ENST00000424542:ENST00000432365 |
| 16:2029621:C:G | | ENST00000424542:ENST00000432365 |
| 16:2029621:C:A | | ENST00000424542:ENST00000432365 |
| 16:2029622:T:C | | ENST00000424542:ENST00000432365 |
| 16:2029625:T:C | | ENST00000424542:ENST00000432365 |
| 16:2029627:G:A | | ENST00000424542:ENST00000432365 |
| 16:2029627:G:T | | ENST00000424542:ENST00000432365 |
| 16:2029627:G:C | rs1183574603 | ENST00000424542:ENST00000432365 |
| 16:2029630:G:T | | ENST00000424542:ENST00000432365 |
| 16:2029630:G:C | rs1446575411 | ENST00000424542:ENST00000432365 |
| 16:2029631:T:C | rs1196088796 | ENST00000424542:ENST00000432365 |
| 16:2029634:A:C | rs1372684231 | ENST00000424542:ENST00000432365 |
| 16:2029635:C:G | | ENST00000424542:ENST00000432365 |
| 16:2029637:A:G | rs1460667729 | ENST00000424542:ENST00000432365 |
| 16:2029639:G:A | | ENST00000424542:ENST00000432365 |
| 16:2029640:A:AG | | ENST00000424542:ENST00000432365 |
| 16:2029640:A:C | rs1397020290 | ENST00000424542:ENST00000432365 |
| 16:2029641:G:C | | ENST00000424542:ENST00000432365 |
| 16:2029642:A:C | | ENST00000424542:ENST00000432365 |
| 16:2029642:A:T | | ENST00000424542:ENST00000432365 |
| 16:2029642:A:G | | ENST00000424542:ENST00000432365 |
| 16:2029643:C:T | rs1334659321 | ENST00000424542:ENST00000432365 |
| 16:2029645:G:A | | ENST00000424542:ENST00000432365 |
| 16:2029645:G:C | | ENST00000424542:ENST00000432365 |
| 16:2029647:T:G | | ENST00000424542:ENST00000432365 |
| 16:2029648:G:A | | ENST00000424542:ENST00000432365 |
| 16:2029651:G:A | | ENST00000424542:ENST00000432365 |
| 16:2029652:A:G | | ENST00000424542:ENST00000432365 |
| 16:2029654:C:T | rs1305334670 | ENST00000424542:ENST00000432365 |
| 16:2029657:C:T | rs573229352 | ENST00000424542:ENST00000432365 |
| 16:2029658:G:T | | ENST00000424542:ENST00000432365 |
| 16:2029658:G:A | rs542224637 | ENST00000424542:ENST00000432365 |
| 16:2029660:C:T | rs922413627 | ENST00000424542:ENST00000432365 |
| 16:2029661:G:C | rs761961644 | ENST00000424542:ENST00000432365 |
| 16:2029661:G:A | rs761961644 | ENST00000424542:ENST00000432365 |
| 16:2029663:C:T | rs555453690 | ENST00000424542:ENST00000432365 |
| 16:2029664:G:A | rs55864883 | ENST00000424542:ENST00000432365 |
| 16:2029664:G:A | rs55864883 | ENST00000424542:ENST00000432365 |
| 16:2029667:A:C | | ENST00000424542:ENST00000432365 |
| 16:2029667:A:T | rs1364445252 | ENST00000424542:ENST00000432365 |
| 16:2029669:C:G | | ENST00000424542:ENST00000432365 |
| 16:2029673:C:T | rs1182457944 | ENST00000424542:ENST00000432365 |
| 16:2029673:C:A | rs1182457944 | ENST00000424542:ENST00000432365 |
| 16:2029675:T:A | | ENST00000424542:ENST00000432365 |
| 16:2029676:G:A | rs1261149226 | ENST00000424542:ENST00000432365 |
| 16:2029676:G:C | | ENST00000424542:ENST00000432365 |
| 16:2029678:A:G | | ENST00000424542:ENST00000432365 |
| 16:2029681:G:A | rs370860382 | ENST00000424542:ENST00000432365 |
| 16:2029687:A:G | | ENST00000563587 |
| 16:2029688:T:C | | ENST00000563587 |
| 16:2029689:G:A | | ENST00000563587 |
| 16:2029689:G:T | rs935753611 | ENST00000563587 |
| 16:2029690:G:A | | ENST00000424542:ENST00000432365:ENST00000563587 |
| 16:2029691:C:G | rs766202541 | ENST00000424542:ENST00000432365:ENST00000563587 |
| 16:2029691:C:T | rs766202541 | ENST00000424542:ENST00000432365:ENST00000563587 |
| 16:2029691:C:A | | ENST00000424542:ENST00000432365:ENST00000563587 |
| 16:2029693:C:A | | ENST00000424542:ENST00000432365:ENST00000563587 |
| 16:2029694:A:G | | ENST00000424542:ENST00000432365:ENST00000563587 |
| 16:2029696:C:G | | ENST00000424542:ENST00000432365:ENST00000563587 |
| 16:2029696:C:T | rs891488832 | ENST00000424542:ENST00000432365:ENST00000563587 |
| 16:2029697:G:A | rs754722358 | ENST00000424542:ENST00000432365:ENST00000563587 |
| 16:2029699:G:A | rs755979092 | ENST00000424542:ENST00000432365:ENST00000563587 |
| 16:2029699:G:T | | ENST00000424542:ENST00000432365:ENST00000563587 |
| 16:2029699:G:C | rs755979092 | ENST00000424542:ENST00000432365:ENST00000563587 |
| 16:2029702:C:CT | | ENST00000424542:ENST00000432365:ENST00000563587 |
| 16:2029703:TC:T | | ENST00000424542:ENST00000432365:ENST00000563587 |
| 16:2029703:TCC:T | rs1295896699 | ENST00000424542:ENST00000432365:ENST00000563587 |
| 16:2029706:C:A | | ENST00000424542:ENST00000432365:ENST00000563587 |
| 16:2029706:C:T | rs752554219 | ENST00000424542:ENST00000432365:ENST00000563587 |
| 16:2029708:C:T | rs758073220 | ENST00000424542:ENST00000432365:ENST00000563587 |
| 16:2029709:C:T | rs777598521 | ENST00000424542:ENST00000432365:ENST00000563587 |
| 16:2029709:C:A | | ENST00000424542:ENST00000432365:ENST00000563587 |
| 16:2029711:G:A | rs373924925 | ENST00000424542:ENST00000432365:ENST00000563587 |
| 16:2029711:G:C | | ENST00000424542:ENST00000432365:ENST00000563587 |
| 16:2029711:G:T | | ENST00000424542:ENST00000432365:ENST00000563587 |
| 16:2029712:C:T | | ENST00000424542:ENST00000432365:ENST00000563587 |
| 16:2029715:A:G | | ENST00000424542:ENST00000432365:ENST00000563587 |
| 16:2029715:A:C | | ENST00000424542:ENST00000432365:ENST00000563587 |
| 16:2029717:G:C | | ENST00000424542:ENST00000432365:ENST00000563587 |
| 16:2029717:G:A | | ENST00000424542:ENST00000432365:ENST00000563587 |
| 16:2029718:A:AC | | ENST00000424542:ENST00000432365:ENST00000563587 |
| 16:2029718:A:T | rs749876642 | ENST00000424542:ENST00000432365:ENST00000563587 |
| 16:2029718:A:C | | ENST00000424542:ENST00000432365:ENST00000563587 |
| 16:2029719:C:A | rs1431176783 | ENST00000424542:ENST00000432365:ENST00000563587 |
| 16:2029720:C:A | | ENST00000424542:ENST00000432365:ENST00000563587 |
| 16:2029720:C:T | rs1414953422 | ENST00000424542:ENST00000432365:ENST00000563587 |
| 16:2029721:C:T | rs1158376575 | ENST00000424542:ENST00000432365:ENST00000563587 |
| 16:2029723:T:C | | ENST00000424542:ENST00000432365:ENST00000563587 |
| 16:2029723:T:A | | ENST00000424542:ENST00000432365:ENST00000563587 |
| 16:2029724:G:A | | ENST00000424542:ENST00000432365:ENST00000563587 |
| 16:2029725:G:A | | ENST00000424542:ENST00000432365:ENST00000563587 |
| 16:2029725:G:C | rs369097244 | ENST00000424542:ENST00000432365:ENST00000563587 |
| 16:2029726:G:A | | ENST00000424542:ENST00000432365:ENST00000563587 |
| 16:2029727:A:T | | ENST00000424542:ENST00000432365:ENST00000563587 |
| 16:2029728:G:T | | ENST00000424542:ENST00000432365:ENST00000563587 |
| 16:2029729:C:G | | ENST00000424542:ENST00000432365:ENST00000563587 |
| 16:2029729:C:A | | ENST00000424542:ENST00000432365:ENST00000563587 |
| 16:2029729:C:T | | ENST00000424542:ENST00000432365:ENST00000563587 |
| 16:2029730:C:T | rs774643224 | ENST00000424542:ENST00000432365:ENST00000563587 |
| 16:2029730:C:G | | ENST00000424542:ENST00000432365:ENST00000563587 |
| 16:2029738:G:C | | ENST00000424542:ENST00000432365:ENST00000563587 |
| 16:2029741:T:A | | ENST00000424542:ENST00000432365:ENST00000563587 |
| 16:2029741:T:G | | ENST00000424542:ENST00000432365:ENST00000563587 |
| 16:2029742:G:T | | ENST00000424542:ENST00000432365:ENST00000563587 |
| 16:2029743:G:C | | ENST00000424542:ENST00000432365:ENST00000563587 |
| 16:2029743:G:T | | ENST00000424542:ENST00000432365:ENST00000563587 |
| 16:2029745:C:G | rs773469231 | ENST00000424542:ENST00000432365:ENST00000563587 |
| 16:2029747:C:T | | ENST00000424542:ENST00000432365:ENST00000563587 |
| 16:2029747:C:CA | | ENST00000424542:ENST00000432365:ENST00000563587 |
| 16:2029748:A:C | | ENST00000424542:ENST00000432365:ENST00000563587 |
| 16:2029748:A:G | rs1315686379 | ENST00000424542:ENST00000432365:ENST00000563587 |
| 16:2029750:A:G | | ENST00000424542:ENST00000432365:ENST00000563587 |
| 16:2029751:C:G | | ENST00000424542:ENST00000432365:ENST00000563587 |
| 16:2029751:C:T | | ENST00000424542:ENST00000432365:ENST00000563587 |
| 16:2029753:G:A | rs540396693 | ENST00000424542:ENST00000432365:ENST00000563587 |
| 16:2029753:G:T | | ENST00000424542:ENST00000432365:ENST00000563587 |
| 16:2029753:G:C | | ENST00000424542:ENST00000432365:ENST00000563587 |
| 16:2029757:G:A | | ENST00000424542:ENST00000432365:ENST00000563587 |
| 16:2029760:A:G | | ENST00000424542:ENST00000432365:ENST00000563587 |
| 16:2029761:C:G | | ENST00000424542:ENST00000432365:ENST00000563587 |
| 16:2029762:A:G | | ENST00000424542:ENST00000432365:ENST00000563587 |
| 16:2029766:C:T | | ENST00000424542:ENST00000432365:ENST00000563587 |
| 16:2029768:G:A | rs753866913 | ENST00000424542:ENST00000432365:ENST00000563587 |
| 16:2029769:A:G | rs559412284 | ENST00000424542:ENST00000432365:ENST00000563587 |
| 16:2029771:G:T | rs765109481 | ENST00000424542:ENST00000432365:ENST00000563587 |
| 16:2029772:C:A | rs1158665549 | ENST00000424542:ENST00000432365:ENST00000563587 |
| 16:2029778:A:G | | ENST00000424542:ENST00000432365:ENST00000563587 |
| 16:2029783:G:A | rs752499334 | ENST00000424542:ENST00000432365:ENST00000563587 |
| 16:2029783:G:T | | ENST00000424542:ENST00000432365:ENST00000563587 |
| 16:2033352:A:G | | ENST00000566198 |
| 16:2033353:T:C | | ENST00000566198 |
| 16:2033356:C:T | rs903781011 | ENST00000566198 |
| 16:2033358:C:G | | ENST00000566198 |
| 16:2033358:C:T | rs745386102 | ENST00000566198 |
| 16:2033359:G:C | | ENST00000566198 |
| 16:2033359:G:T | | ENST00000566198 |
| 16:2033359:G:A | rs556062695 | ENST00000566198 |
| 16:2033362:C:G | | ENST00000566198 |
| 16:2033364:G:T | | ENST00000566198 |
| 16:2033364:G:A | rs575900421 | ENST00000566198 |
| 16:2033368:G:A | | ENST00000566198 |
| 16:2033370:G:A | rs995535875 | ENST00000566198 |
| 16:2033371:C:T | rs1342635787 | ENST00000566198 |
| 16:2033374:C:T | rs1013327982 | ENST00000566198 |
| 16:2033374:C:G | | ENST00000566198 |
| 16:2033376:C:A | rs1202163114 | ENST00000566198 |
| 16:2033380:C:G | | ENST00000566198 |
| 16:2033380:C:T | rs544837548 | ENST00000566198 |
| 16:2033382:G:T | | ENST00000566198 |
| 16:2033383:C:T | rs773415998 | ENST00000566198 |
| 16:2033385:C:T | rs565069068 | ENST00000566198 |
| 16:2033386:G:A | rs995594330 | ENST00000566198 |
| 16:2033388:G:A | | ENST00000566198 |
| 16:2033391:C:G | rs1477959662 | ENST00000566198 |
| 16:2033392:C:T | rs1027216418 | ENST00000566198 |
| 16:2033392:C:A | | ENST00000566198 |
| 16:2033394:G:A | | ENST00000566198 |
| 16:2033397:G:C | | ENST00000566198 |
| 16:2033397:G:A | | ENST00000566198 |
| 16:2033400:C:G | | ENST00000566198 |
| 16:2033403:C:G | rs1468699332 | ENST00000566198 |
| 16:2033403:C:T | | ENST00000566198 |
| 16:2033406:C:G | | ENST00000566198 |
| 16:2033406:C:T | | ENST00000566198 |
| 16:2033407:G:A | rs951614919 | ENST00000566198 |
| 16:2033410:G:A | | ENST00000566198 |
| 16:2033410:G:T | | ENST00000566198 |
| 16:2033412:C:T | rs1402973805 | ENST00000566198 |
| 16:2033413:C:T | | ENST00000566198 |
| 16:2033415:C:T | rs776721657 | ENST00000566198 |
| 16:2033418:C:A | | ENST00000566198 |
| 16:2033418:C:T | rs759565734 | ENST00000566198 |
| 16:2033419:A:G | | ENST00000566198 |
| 16:2033421:A:G | | ENST00000566198 |
| 16:2033422:G:T | | ENST00000566198 |
| 16:2033423:G:C | | ENST00000566198 |
| 16:2033425:T:G | | ENST00000566198 |
| 16:2033427:G:A | | ENST00000566198 |
| 16:2033430:C:T | rs958526665 | ENST00000566198 |
| 16:2033432:G:C | | ENST00000566198 |
| 16:2033433:G:A | rs982916470 | ENST00000566198 |
| 16:2033433:G:T | | ENST00000566198 |
| 16:2033434:T:C | | ENST00000566198 |
| 16:2036324:G:C | | ENST00000424542:ENST00000432365: |
| | | ENST00000563587:ENST00000566198 |
| 16:2036324:G:T | rs946899064 | ENST00000424542:ENST00000432365: |
| | | ENST00000563587:ENST00000566198 |
| 16:2036327:G:A | rs375612309 | ENST00000424542:ENST00000432365: |
| | | ENST00000563587:ENST00000566198 |
| 16:2036328:T:A | | ENST00000424542:ENST00000432365: |
| | | ENST00000563587:ENST00000566198 |
| 16:2036329:C:CA | | ENST00000424542:ENST00000432365: |
| | | ENST00000563587:ENST00000566198 |
| 16:2036330:A:C | | ENST00000424542:ENST00000432365: |
| | | ENST00000563587:ENST00000566198 |
| 16:2036330:A:G | rs774149996 | ENST00000424542:ENST00000432365: |
| | | ENST00000563587:ENST00000566198 |
| 16:2036331:G:A | | ENST00000424542:ENST00000432365: |
| | | ENST00000563587:ENST00000566198 |
| 16:2036331:G:C | rs368296720 | ENST00000424542:ENST00000432365: |
| | | ENST00000563587:ENST00000566198 |
| 16:2036331:G:T | | ENST00000424542:ENST00000432365: |
| | | ENST00000563587:ENST00000566198 |
| 16:2036333:G:T | | ENST00000424542:ENST00000432365: |
| | | ENST00000563587:ENST00000566198 |
| 16:2036334:G:A | | ENST00000424542:ENST00000432365: |
| | | ENST00000563587:ENST00000566198 |
| 16:2036334:G:C | | ENST00000424542:ENST00000432365: |
| | | ENST00000563587:ENST00000566198 |
| 16:2036336:C:T | rs1377584895 | ENST00000424542:ENST00000432365: |
| | | ENST00000563587:ENST00000566198 |
| 16:2036336:C:A | | ENST00000424542:ENST00000432365: |
| | | ENST00000563587:ENST00000566198 |
| 16:2036337:C:T | rs571876690 | ENST00000424542:ENST00000432365: |
| | | ENST00000563587:ENST00000566198 |
| 16:2036338:CCT:C | rs753860117 | ENST00000424542:ENST00000432365: |
| | | ENST00000563587:ENST00000566198 |
| 16:2036340:T:C | rs750097832 | ENST00000424542:ENST00000432365: |
| | | ENST00000563587:ENST00000566198 |
| 16:2036340:T:G | | ENST00000424542:ENST00000432365: |
| | | ENST00000563587:ENST00000566198 |
| 16:2036342:A:G | | ENST00000424542:ENST00000432365: |
| | | ENST00000563587:ENST00000566198 |
| 16:2036343:G:A | | ENST00000424542:ENST00000432365: |
| | | ENST00000563587:ENST00000566198 |
| 16:2036343:G:T | | ENST00000424542:ENST00000432365: |
| | | ENST00000563587:ENST00000566198 |
| 16:2036345:G:A | rs765806585 | ENST00000424542:ENST00000432365: |
| | | ENST00000563587:ENST00000566198 |
| 16:2036347:G:C | rs1266396088 | ENST00000424542:ENST00000432365: |
| | | ENST00000563587:ENST00000566198 |
| 16:2036348:C:A | | ENST00000424542:ENST00000432365: |
| | | ENST00000563587:ENST00000566198 |
| 16:2036351:C:A | | ENST00000424542:ENST00000432365: |
| | | ENST00000563587:ENST00000566198 |
| 16:2036351:C:T | rs375328638 | ENST00000424542:ENST00000432365: |
| | | ENST00000563587:ENST00000566198 |
| 16:2036352:G:A | rs201388997 | ENST00000424542:ENST00000432365: |
| | | ENST00000563587:ENST00000566198 |
| 16:2036352:G:A | rs201388997 | ENST00000424542:ENST00000432365: |
| | | ENST00000563587:ENST00000566198 |
| 16:2036354:C:T | | ENST00000424542:ENST00000432365: |
| | | ENST00000563587:ENST00000566198 |
| 16:2036355:C:T | rs747367425 | ENST00000424542:ENST00000432365: |
| | | ENST00000563587:ENST00000566198 |
| 16:2036357:C:G | rs757611488 | ENST00000424542:ENST00000432365: |
| | | ENST00000563587:ENST00000566198 |
| 16:2036357:C:T | rs757611488 | ENST00000424542:ENST00000432365: |
| | | ENST00000563587:ENST00000566198 |
| 16:2036358:G:A | rs781464941 | ENST00000424542:ENST00000432365: |
| | | ENST00000563587:ENST00000566198 |
| 16:2036360:C:T | rs202089731 | ENST00000424542:ENST00000432365: |
| | | ENST00000563587:ENST00000566198 |
| 16:2036361:T:C | | ENST00000424542:ENST00000432365: |
| | | ENST00000563587:ENST00000566198 |
| 16:2036366:C:A | | ENST00000424542:ENST00000432365: |
| | | ENST00000563587:ENST00000566198 |
| 16:2036368:C:G | rs1463823367 | ENST00000424542:ENST00000432365: |
| | | ENST00000563587:ENST00000566198 |
| 16:2036370:T:C | rs1400863425 | ENST00000424542:ENST00000432365: |
| | | ENST00000563587:ENST00000566198 |
| 16:2036372:C:T | rs374728713 | ENST00000424542:ENST00000432365: |
| | | ENST00000563587:ENST00000566198 |
| 16:2036372:C:G | | ENST00000424542:ENST00000432365: |
| | | ENST00000563587:ENST00000566198 |
| 16:2036372:C:T | rs374728713 | ENST00000424542:ENST00000432365: |
| | | ENST00000563587:ENST00000566198 |
| 16:2036373:G:A | rs369991176 | ENST00000424542:ENST00000432365: |
| | | ENST00000563587:ENST00000566198 |
| 16:2036373:G:C | rs369991176 | ENST00000424542:ENST00000432365: |
| | | ENST00000563587:ENST00000566198 |
| 16:2036378:G:A | rs774199613 | ENST00000424542:ENST00000432365: |
| | | ENST00000563587:ENST00000566198 |
| 16:2036379:G:C | | ENST00000424542:ENST00000432365: |
| | | ENST00000563587:ENST00000566198 |
| 16:2036379:G:A | rs963180690 | ENST00000424542:ENST00000432365: |
| | | ENST00000563587:ENST00000566198 |
| 16:2036380:A:ACCTCAGG | | ENST00000424542:ENST00000432365: |
| | | ENST00000563587:ENST00000566198 |
| 16:2036382:C:T | rs761651739 | ENST00000424542:ENST00000432365: |
| | | ENST00000563587:ENST00000566198 |
| 16:2036384:CAG:C | | ENST00000424542:ENST00000432365: |
| | | ENST00000563587:ENST00000566198 |
| 16:2036386:G:C | | ENST00000424542:ENST00000432365: |
| | | ENST00000563587:ENST00000566198 |
| 16:2036387:G:A | rs773061394 | ENST00000424542:ENST00000432365: |
| | | ENST00000563587:ENST00000566198 |
| 16:2036387:G:C | | ENST00000424542:ENST00000432365: |
| | | ENST00000563587:ENST00000566198 |
| 16:2036388:G:T | | ENST00000424542:ENST00000432365: |
| | | ENST00000563587:ENST00000566198 |
| 16:2036390:T:C | rs1260401474 | ENST00000424542:ENST00000432365: |
| | | ENST00000563587:ENST00000566198 |
| 16:2036391:A:G | rs1319140101 | ENST00000424542:ENST00000432365: |
| | | ENST00000563587:ENST00000566198 |
| 16:2036393:G:T | | ENST00000424542:ENST00000432365: |
| | | ENST00000563587:ENST00000566198 |
| 16:2036393:G:A | rs1203647635 | ENST00000424542:ENST00000432365: |
| | | ENST00000563587:ENST00000566198 |
| 16:2036396:T:C | | ENST00000424542:ENST00000432365: |
| | | ENST00000563587:ENST00000566198 |
| 16:2036399:A:C | | ENST00000424542:ENST00000432365: |
| | | ENST00000563587:ENST00000566198 |
| 16:2036400:A:T | | ENST00000424542:ENST00000432365: |
| | | ENST00000563587:ENST00000566198 |
| 16:2036403:T:G | | ENST00000424542:ENST00000432365: |
| | | ENST00000563587:ENST00000566198 |
| 16:2036405:CAT:C | | ENST00000424542:ENST00000432365: |
| | | ENST00000563587:ENST00000566198 |
| 16:2036406:A:G | rs1261251553 | ENST00000424542:ENST00000432365: |
| | | ENST00000563587:ENST00000566198 |
| 16:2036408:A:G | | ENST00000424542:ENST00000432365: |
| | | ENST00000563587:ENST00000566198 |
| 16:2036409:G:C | rs759086094 | ENST00000424542:ENST00000432365: |
| | | ENST00000563587:ENST00000566198 |
| 16:2036410:T:G | rs764639092 | ENST00000424542:ENST00000432365: |
| | | ENST00000563587:ENST00000566198 |
| 16:2036411:G:A | | ENST00000424542:ENST00000432365: |
| | | ENST00000563587:ENST00000566198 |
| 16:2036412:A:T | rs34634388 | ENST00000424542:ENST00000432365: |
| | | ENST00000563587:ENST00000566198 |
| 16:2036412:A:G | | ENST00000424542:ENST00000432365: |
| | | ENST00000563587:ENST00000566198 |
| 16:2036413:C:G | rs757636827 | ENST00000424542:ENST00000432365: |
| | | ENST00000563587:ENST00000566198 |
| 16:2036415:A:G | | ENST00000424542:ENST00000432365: |
| | | ENST00000563587:ENST00000566198 |
| 16:2036415:A:T | | ENST00000424542:ENST00000432365: |
| | | ENST00000563587:ENST00000566198 |
| 16:2036415:A:C | rs781345872 | ENST00000424542:ENST00000432365: |
| | | ENST00000563587:ENST00000566198 |
| 16:2036416:G:T | | ENST00000424542:ENST00000432365: |
| | | ENST00000563587:ENST00000566198 |
| 16:2036417:T:C | rs776205113 | ENST00000424542:ENST00000432365: |
| | | ENST00000563587:ENST00000566198 |
| 16:2036417:T:G | | ENST00000424542:ENST00000432365: |
| | | ENST00000563587:ENST00000566198 |
| 16:2036418:C:T | | ENST00000424542:ENST00000432365: |
| | | ENST00000563587:ENST00000566198 |
| 16:2036420:C:T | rs139491786 | ENST00000424542:ENST00000432365: |
| | | ENST00000563587:ENST00000566198 |
| 16:2036420:C:T | rs139491786 | ENST00000424542:ENST00000432365: |
| | | ENST00000563587:ENST00000566198 |
| 16:2036421:G:T | | ENST00000424542:ENST00000432365: |
| | | ENST00000563587:ENST00000566198 |
| 16:2036421:G:A | rs555526303 | ENST00000424542:ENST00000432365: |
| | | ENST00000563587:ENST00000566198 |
| 16:2036421:G:C | rs555526303 | ENST00000424542:ENST00000432365: |
| | | ENST00000563587:ENST00000566198 |
| 16:2036423:C:T | rs185371475 | ENST00000424542:ENST00000432365: |
| | | ENST00000563587:ENST00000566198 |
| 16:2036423:C:T | rs185371475 | ENST00000424542:ENST00000432365: |
| | | ENST00000563587:ENST00000566198 |
| 16:2036426:G:A | rs199574930 | ENST00000424542:ENST00000432365: |
| | | ENST00000563587:ENST00000566198 |
| 16:2036426:G:T | | ENST00000424542:ENST00000432365: |
| | | ENST00000563587:ENST00000566198 |
| 16:2036427:G:A | rs1299758672 | ENST00000424542:ENST00000432365: |
| | | ENST00000563587:ENST00000566198 |
| 16:2036427:G:C | | ENST00000424542:ENST00000432365: |
| | | ENST00000563587:ENST00000566198 |
| 16:2036431:G:C | | ENST00000424542:ENST00000432365: |
| | | ENST00000563587:ENST00000566198 |
| 16:2036432:T:C | | ENST00000424542:ENST00000432365: |
| | | ENST00000563587:ENST00000566198 |
| 16:2036435:A:T | | ENST00000424542:ENST00000432365: |
| | | ENST00000563587:ENST00000566198 |
| 16:2036436:T:C | | ENST00000424542:ENST00000432365: |
| | | ENST00000563587:ENST00000566198 |
| 16:2036438:C:T | rs772830453 | ENST00000424542:ENST00000432365: |
| | | ENST00000563587:ENST00000566198 |
| 16:2036439:G:A | rs760492388 | ENST00000424542:ENST00000432365: |
| | | ENST00000563587:ENST00000566198 |
| 16:2036442:C:G | | ENST00000424542:ENST00000432365: |
| | | ENST00000563587:ENST00000566198 |
| 16:2036444:G:A | | ENST00000424542:ENST00000432365: |
| | | ENST00000563587:ENST00000566198 |
| 16:2036445:T:G | | ENST00000424542:ENST00000432365: |
| | | ENST00000563587:ENST00000566198 |
| 16:2036445:T:C | | ENST00000424542:ENST00000432365: |
| | | ENST00000563587:ENST00000566198 |
| 16:2036447:G:C | | ENST00000424542:ENST00000432365: |
| | | ENST00000563587:ENST00000566198 |
| 16:2036448:A:C | | ENST00000424542:ENST00000432365: |
| | | ENST00000563587:ENST00000566198 |
| 16:2036450:C:G | rs34922370 | ENST00000424542:ENST00000432365: |
| | | ENST00000563587:ENST00000566198 |
| 16:2036450:C:T | | ENST00000424542:ENST00000432365: |
| | | ENST00000563587:ENST00000566198 |
| 16:2036451:C:G | rs374997543 | ENST00000424542:ENST00000432365: |
| | | ENST00000563587:ENST00000566198 |
| 16:2036451:C:T | rs374997543 | ENST00000424542:ENST00000432365: |
| | | ENST00000563587:ENST00000566198 |
| 16:2036451:C:A | | ENST00000424542:ENST00000432365: |
| | | ENST00000563587:ENST00000566198 |
| 16:2036453:G:A | rs1180615296 | ENST00000424542:ENST00000432365: |
| | | ENST00000563587:ENST00000566198 |
| 16:2036457:C:T | rs756591539 | ENST00000424542:ENST00000432365: |
| | | ENST00000563587:ENST00000566198 |
| 16:2036457:C:G | | ENST00000424542:ENST00000432365: |
| | | ENST00000563587:ENST00000566198 |
| 16:2036459:C:T | | ENST00000424542:ENST00000432365: |
| | | ENST00000563587:ENST00000566198 |
| 16:2036460:C:T | | ENST00000424542:ENST00000432365: |
| | | ENST00000563587:ENST00000566198 |
| 16:2036460:C:G | rs560139324 | ENST00000424542:ENST00000432365: |
| | | ENST00000563587:ENST00000566198 |
| 16:2036465:G:A | rs367680143 | ENST00000424542:ENST00000432365: |
| | | ENST00000563587:ENST00000566198 |
| 16:2036466:C:T | rs755229198 | ENST00000424542:ENST00000432365: |
| | | ENST00000563587:ENST00000566198 |
| 16:2036468:C:T | rs62038800 | ENST00000424542:ENST00000432365: |
| | | ENST00000563587:ENST00000566198 |
| 16:2036468:C:T | rs62038800 | ENST00000424542:ENST00000432365: |
| | | ENST00000563587:ENST00000566198 |
| 16:2036468:C:G | rs62038800 | ENST00000424542:ENST00000432365: |
| | | ENST00000563587:ENST00000566198 |
| 16:2036469:G:C | | ENST00000424542:ENST00000432365: |
| | | ENST00000563587:ENST00000566198 |
| 16:2036469:G:A | rs548076995 | ENST00000424542:ENST00000432365: |
| | | ENST00000563587:ENST00000566198 |
| 16:2036472:C:G | | ENST00000424542:ENST00000432365: |
| | | ENST00000563587:ENST00000566198 |
| 16:2036474:G:A | | ENST00000424542:ENST00000432365: |
| | | ENST00000563587:ENST00000566198 |
| 16:2036475:G:A | rs1286946608 | ENST00000424542:ENST00000432365: |
| | | ENST00000563587:ENST00000566198 |
| 16:2036480:C:A | rs200865634 | ENST00000424542:ENST00000432365: |
| | | ENST00000563587:ENST00000566198 |
| 16:2036480:C:G | rs200865634 | ENST00000424542:ENST00000432365: |
| | | ENST00000563587:ENST00000566198 |
| 16:2036480:C:T | rs200865634 | ENST00000424542:ENST00000432365: |
| | | ENST00000563587:ENST00000566198 |
| 16:2036480:C:G | rs200865634 | ENST00000424542:ENST00000432365: |
| | | ENST00000563587:ENST00000566198 |
| 16:2036481:G:A | rs202019612 | ENST00000424542:ENST00000432365: |
| | | ENST00000563587:ENST00000566198 |
| 16:2036481:G:T | | ENST00000424542:ENST00000432365: |
| | | ENST00000563587:ENST00000566198 |
| 16:2036481:G:A | rs202019612 | ENST00000424542:ENST00000432365: |
| | | ENST00000563587:ENST00000566198 |
| 16:2036483:G:T | rs200328877 | ENST00000424542:ENST00000432365: |
| | | ENST00000563587:ENST00000566198 |
| 16:2036483:G:A | rs200328877 | ENST00000424542:ENST00000432365: |
| | | ENST00000563587:ENST00000566198 |
| 16:2036484:C:G | | ENST00000424542:ENST00000432365: |
| | | ENST00000563587:ENST00000566198 |
| 16:2036484:C:T | rs1008675540 | ENST00000424542:ENST00000432365: |
| | | ENST00000563587:ENST00000566198 |
| 16:2036486:C:A | rs762558620 | ENST00000424542:ENST00000432365: |
| | | ENST00000563587:ENST00000566198 |
| 16:2036489:G:A | | ENST00000424542:ENST00000432365: |
| | | ENST00000563587:ENST00000566198 |
| 16:2036489:G:C | | ENST00000424542:ENST00000432365: |
| | | ENST00000563587:ENST00000566198 |
| 16:2036490:A:C | | ENST00000424542:ENST00000432365: |
| | | ENST00000563587:ENST00000566198 |
| 16:2036490:A:T | | ENST00000424542:ENST00000432365: |
| | | ENST00000563587:ENST00000566198 |
| 16:2036492:C:T | rs371625359 | ENST00000424542:ENST00000432365: |
| | | ENST00000563587:ENST00000566198 |
| 16:2036493:G:A | rs773908897 | ENST00000424542:ENST00000432365: |
| | | ENST00000563587:ENST00000566198 |
| 16:2036493:G:C | | ENST00000424542:ENST00000432365: |
| | | ENST00000563587:ENST00000566198 |
| 16:2036495:C:A | | ENST00000424542:ENST00000432365: |
| | | ENST00000563587:ENST00000566198 |
| 16:2036495:C:G | | ENST00000424542:ENST00000432365: |
| | | ENST00000563587:ENST00000566198 |
| 16:2036498:A:G | rs11544568 | ENST00000424542:ENST00000432365: |
| | | ENST00000563587:ENST00000566198 |
| 16:2036499:T:C | | ENST00000424542:ENST00000432365: |
| | | ENST00000563587:ENST00000566198 |
| 16:2036500:T:G | | ENST00000424542:ENST00000432365: |
| | | ENST00000563587:ENST00000566198 |
| 16:2036501:G:C | rs368902168 | ENST00000424542:ENST00000432365: |
| | | ENST00000563587:ENST00000566198 |
| 16:2036503:G:C | | ENST00000424542:ENST00000432365: |
| | | ENST00000563587:ENST00000566198 |
| 16:2036504:G:A | | ENST00000424542:ENST00000432365: |
| | | ENST00000563587:ENST00000566198 |
| 16:2036505:T:C | | ENST00000424542:ENST00000432365: |
| | | ENST00000563587:ENST00000566198 |
| 16:2036727:G:C | | ENST00000424542:ENST00000432365: |
| | | ENST00000563587:ENST00000566198 |
| 16:2036728:G:A | rs1220601920 | ENST00000424542:ENST00000432365: |
| | | ENST00000563587:ENST00000566198 |
| 16:2036729:T:C | rs771530557 | ENST00000424542:ENST00000432365: |
| | | ENST00000563587:ENST00000566198 |
| 16:2036732:A:C | | ENST00000424542:ENST00000432365: |
| | | ENST00000563587:ENST00000566198 |
| 16:2036732:A:G | | ENST00000424542:ENST00000432365: |
| | | ENST00000563587:ENST00000566198 |
| 16:2036734:G:A | rs770260353 | ENST00000424542:ENST00000432365: |
| | | ENST00000563587:ENST00000566198 |
| 16:2036734:G:C | | ENST00000424542:ENST00000432365: |
| | | ENST00000563587:ENST00000566198 |
| 16:2036734:G:T | | ENST00000424542:ENST00000432365: |
| | | ENST00000563587:ENST00000566198 |
| 16:2036735:G:A | | ENST00000424542:ENST00000432365: |
| | | ENST00000563587:ENST00000566198 |
| 16:2036737:C:A | | ENST00000424542:ENST00000432365: |
| | | ENST00000563587:ENST00000566198 |
| 16:2036740:A:T | | ENST00000424542:ENST00000432365: |
| | | ENST00000563587:ENST00000566198 |
| 16:2036743:G:T | | ENST00000424542:ENST00000432365: |
| | | ENST00000563587:ENST00000566198 |
| 16:2036743:G:C | | ENST00000424542:ENST00000432365: |
| | | ENST00000563587:ENST00000566198 |
| 16:2036744:T:C | | ENST00000424542:ENST00000432365: |
| | | ENST00000563587:ENST00000566198 |
| 16:2036746:G:A | rs370378663 | ENST00000424542:ENST00000432365: |
| | | ENST00000563587:ENST00000566198 |
| 16:2036748:G:C | | ENST00000424542:ENST00000432365: |
| | | ENST00000563587:ENST00000566198 |
| 16:2036749:G:A | | ENST00000424542:ENST00000432365: |
| | | ENST00000563587:ENST00000566198 |
| 16:2036749:G:T | rs759144405 | ENST00000424542:ENST00000432365: |
| | | ENST00000563587:ENST00000566198 |
| 16:2036750:G:T | | ENST00000424542:ENST00000432365: |
| | | ENST00000563587:ENST00000566198 |
| 16:2036752:C:G | | ENST00000424542:ENST00000432365: |
| | | ENST00000563587:ENST00000566198 |
| 16:2036755:C:T | rs767422892 | ENST00000424542:ENST00000432365: |
| | | ENST00000563587:ENST00000566198 |
| 16:2036756:G:A | rs376982217 | ENST00000424542:ENST00000432365: |
| | | ENST00000563587:ENST00000566198 |
| 16:2036756:G:C | | ENST00000424542:ENST00000432365: |
| | | ENST00000563587:ENST00000566198 |
| 16:2036756:G:T | | ENST00000424542:ENST00000432365: |
| | | ENST00000563587:ENST00000566198 |
| 16:2036758:C:A | | ENST00000424542:ENST00000432365: |
| | | ENST00000563587:ENST00000566198 |
| 16:2036761:G:A | rs1352389868 | ENST00000424542:ENST00000432365: |
| | | ENST00000563587:ENST00000566198 |
| 16:2036765:A:T | | ENST00000424542:ENST00000432365: |
| | | ENST00000563587:ENST00000566198 |
| 16:2036766:G:T | | ENST00000424542:ENST00000432365: |
| | | ENST00000563587:ENST00000566198 |
| 16:2036766:G:C | rs1287656643 | ENST00000424542:ENST00000432365: |
| | | ENST00000563587:ENST00000566198 |
| 16:2036767:G:A | rs1353979424 | ENST00000424542:ENST00000432365: |
| | | ENST00000563587:ENST00000566198 |
| 16:2036768:T:C | rs779835809 | ENST00000424542:ENST00000432365: |
| | | ENST00000563587:ENST00000566198 |
| 16:2036771:T:G | | ENST00000424542:ENST00000432365: |
| | | ENST00000563587:ENST00000566198 |
| 16:2036773:G:A | | ENST00000424542:ENST00000432365: |
| | | ENST00000563587:ENST00000566198 |
| 16:2036773:G:T | | ENST00000424542:ENST00000432365: |
| | | ENST00000563587:ENST00000566198 |
| 16:2036774:C:G | | ENST00000424542:ENST00000432365: |
| | | ENST00000563587:ENST00000566198 |
| 16:2036774:C:A | | ENST00000424542:ENST00000432365: |
| | | ENST00000563587:ENST00000566198 |
| 16:2036774:C:T | | ENST00000424542:ENST00000432365: |
| | | ENST00000563587:ENST00000566198 |
| 16:2036777:G:T | | ENST00000424542:ENST00000432365: |
| | | ENST00000563587:ENST00000566198 |
| 16:2036777:G:C | | ENST00000424542:ENST00000432365: |
| | | ENST00000563587:ENST00000566198 |
| 16:2036780:T:C | | ENST00000424542:ENST00000432365: |
| | | ENST00000563587:ENST00000566198 |
| 16:2036783:A:G | rs753593818 | ENST00000424542:ENST00000432365: |
| | | ENST00000563587:ENST00000566198 |
| 16:2036784:G:C | rs754633319 | ENST00000424542:ENST00000432365: |
| | | ENST00000563587:ENST00000566198 |
| 16:2036785:G:C | | ENST00000424542:ENST00000432365: |
| | | ENST00000563587:ENST00000566198 |
| 16:2036786:C:T | | ENST00000424542:ENST00000432365: |
| | | ENST00000563587:ENST00000566198 |
| 16:2036786:C:CACGGGAGG | | ENST00000424542:ENST00000432365: |
| | | ENST00000563587:ENST00000566198 |
| 16:2036786:C:A | | ENST00000424542:ENST00000432365: |
| | | ENST00000563587:ENST00000566198 |
| 16:2036788:C:T | rs778746050 | ENST00000424542:ENST00000432365: |
| | | ENST00000563587:ENST00000566198 |
| 16:2036789:G:A | rs192036446 | ENST00000424542:ENST00000432365: |
| | | ENST00000563587:ENST00000566198 |
| 16:2036791:G:A | rs374420788 | ENST00000424542:ENST00000432365: |
| | | ENST00000563587:ENST00000566198 |
| 16:2036791:G:C | rs374420788 | ENST00000424542:ENST00000432365: |
| | | ENST00000563587:ENST00000566198 |
| 16:2036794:G:A | | ENST00000424542:ENST00000432365: |
| | | ENST00000563587:ENST00000566198 |
| 16:2036797:G:A | rs57986628 | ENST00000424542:ENST00000432365: |
| | | ENST00000563587:ENST00000566198 |
| 16:2036797:G:A | rs57986628 | ENST00000424542:ENST00000432365: |
| | | ENST00000563587:ENST00000566198 |
| 16:2036799:G:C | rs770216899 | ENST00000424542:ENST00000432365: |
| | | ENST00000563587:ENST00000566198 |
| 16:2036800:G:A | rs775875085 | ENST00000424542:ENST00000432365: |
| | | ENST00000563587:ENST00000566198 |
| 16:2036803:C:T | rs958340418 | ENST00000424542:ENST00000432365: |
| | | ENST00000563587:ENST00000566198 |
| 16:2036804:G:A | rs371739183 | ENST00000424542:ENST00000432365: |
| | | ENST00000563587:ENST00000566198 |
| 16:2036806:C:A | | ENST00000424542:ENST00000432365: |
| | | ENST00000563587:ENST00000566198 |
| 16:2036809:C:A | | ENST00000424542:ENST00000432365: |
| | | ENST00000563587:ENST00000566198 |
| 16:2036812:G:A | | ENST00000424542:ENST00000432365: |
| | | ENST00000563587:ENST00000566198 |
| 16:2036812:G:C | rs991543944 | ENST00000424542:ENST00000432365: |
| | | ENST00000563587:ENST00000566198 |
| 16:2036815:G:A | rs774608361 | ENST00000424542:ENST00000432365: |
| | | ENST00000563587:ENST00000566198 |
| 16:2036815:G:C | | ENST00000424542:ENST00000432365: |
| | | ENST00000563587:ENST00000566198 |
| 16:2036820:C:A | | ENST00000424542:ENST00000432365: |
| | | ENST00000563587:ENST00000566198 |
| 16:2036821:C:A | | ENST00000424542:ENST00000432365: |
| | | ENST00000563587:ENST00000566198 |
| 16:2036824:G:A | rs766255635 | ENST00000424542:ENST00000432365: |
| | | ENST00000563587:ENST00000566198 |
| 16:2036825:A:G | rs753633799 | ENST00000424542:ENST00000432365: |
| | | ENST00000563587:ENST00000566198 |
| 16:2036826:G:C | rs374055389 | ENST00000424542:ENST00000432365: |
| | | ENST00000563587:ENST00000566198 |
| 16:2036827:A:G | | ENST00000424542:ENST00000432365: |
| | | ENST00000563587:ENST00000566198 |
| 16:2036828:C:T | | ENST00000424542:ENST00000432365: |
| | | ENST00000563587:ENST00000566198 |
| 16:2036828:C:G | | ENST00000424542:ENST00000432365: |
| | | ENST00000563587:ENST00000566198 |
| 16:2036830:G:C | | ENST00000424542:ENST00000432365: |
| | | ENST00000563587:ENST00000566198 |
| 16:2036830:G:A | | ENST00000424542:ENST00000432365: |
| | | ENST00000563587:ENST00000566198 |
| 16:2036832:T:A | rs1187153627 | ENST00000424542:ENST00000432365: |
| | | ENST00000563587:ENST00000566198 |
| 16:2036836:C:T | | ENST00000424542:ENST00000432365: |
| | | ENST00000563587:ENST00000566198 |
| 16:2036837:A:G | rs1459204762 | ENST00000424542:ENST00000432365: |
| | | ENST00000563587:ENST00000566198 |
| 16:2036838:C:G | | ENST00000424542:ENST00000432365: |
| | | ENST00000563587:ENST00000566198 |
| 16:2036839:T:C | | ENST00000424542:ENST00000432365: |
| | | ENST00000563587:ENST00000566198 |
| 16:2036842:A:G | | ENST00000424542:ENST00000432365: |
| | | ENST00000563587:ENST00000566198 |
| 16:2036843:A:G | | ENST00000424542:ENST00000432365: |
| | | ENST00000563587:ENST00000566198 |
| 16:2036845:C:T | rs752462608 | ENST00000424542:ENST00000432365: |
| | | ENST00000563587:ENST00000566198 |
| 16:2036846:G:A | rs41292275 | ENST00000424542:ENST00000432365: |
| | | ENST00000563587:ENST00000566198 |
| 16:2036846:G:A | rs41292275 | ENST00000424542:ENST00000432365: |
| | | ENST00000563587:ENST00000566198 |
| 16:2036848:C:T | rs777279949 | ENST00000424542:ENST00000432365: |
| | | ENST00000563587:ENST00000566198 |
| 16:2036851:C:T | rs200374798 | ENST00000424542:ENST00000432365: |
| | | ENST00000563587:ENST00000566198 |
| 16:2036852:G:C | rs558590446 | ENST00000424542:ENST00000432365: |
| | | ENST00000563587:ENST00000566198 |
| 16:2036852:G:A | rs558590446 | ENST00000424542:ENST00000432365: |
| | | ENST00000563587:ENST00000566198 |
| 16:2036853:G:GGTCA | | ENST00000424542:ENST00000432365: |
| | | ENST00000563587:ENST00000566198 |
| 16:2036854:G:T | rs1303893616 | ENST00000424542:ENST00000432365: |
| | | ENST00000563587:ENST00000566198 |
| 16:2036855:T:C | rs749632530 | ENST00000424542:ENST00000432365: |
| | | ENST00000563587:ENST00000566198 |
| 16:2036860:C:G | | ENST00000424542:ENST00000432365: |
| | | ENST00000563587:ENST00000566198 |
| 16:2036866:G:A | rs368358326 | ENST00000424542:ENST00000432365: |
| | | ENST00000563587:ENST00000566198 |
| 16:2036866:G:C | | ENST00000424542:ENST00000432365: |
| | | ENST00000563587:ENST00000566198 |
| 16:2036869:G:A | rs369523760 | ENST00000424542:ENST00000432365: |
| | | ENST00000563587:ENST00000566198 |
| 16:2036870:A:G | rs1023795929 | ENST00000424542:ENST00000432365: |
| | | ENST00000563587:ENST00000566198 |
| 16:2036872:C:T | rs969521169 | ENST00000424542:ENST00000432365: |
| | | ENST00000563587:ENST00000566198 |
| 16:2036874:C:CGTGGAAG | | ENST00000424542:ENST00000432365: |
| | | ENST00000563587:ENST00000566198 |
| 16:2036875:G:A | rs764685291 | ENST00000424542:ENST00000432365: |
| | | ENST00000563587:ENST00000566198 |
| 16:2036875:G:C | | ENST00000424542:ENST00000432365: |
| | | ENST00000563587:ENST00000566198 |
| 16:2036876:T:C | rs760634774 | ENST00000424542:ENST00000432365: |
| | | ENST00000563587:ENST00000566198 |
| 16:2036878:G:A | | ENST00000424542:ENST00000432365: |
| | | ENST00000563587:ENST00000566198 |
| 16:2036879:A:C | | ENST00000424542:ENST00000432365: |
| | | ENST00000563587:ENST00000566198 |
| 16:2036880:A:C | | ENST00000424542:ENST00000432365: |
| | | ENST00000563587:ENST00000566198 |
| 16:2036955:G:T | rs1218450587 | ENST00000424542:ENST00000432365: |
| | | ENST00000563587:ENST00000566198 |
| 16:2036957:C:A | | ENST00000424542:ENST00000432365: |
| | | ENST00000563587:ENST00000566198 |
| 16:2036958:C:T | rs567271105 | ENST00000424542:ENST00000432365: |
| | | ENST00000563587:ENST00000566198 |
| 16:2036958:C:G | rs567271105 | ENST00000424542:ENST00000432365: |
| | | ENST00000563587:ENST00000566198 |
| 16:2036961:T:C | rs1268809337 | ENST00000424542:ENST00000432365: |
| | | ENST00000563587:ENST00000566198 |
| 16:2036962:GCCGTCAC:G | | ENST00000424542:ENST00000432365: |
| | | ENST00000563587:ENST00000566198 |
| 16:2036963:C:G | | ENST00000424542:ENST00000432365: |
| | | ENST00000563587:ENST00000566198 |
| 16:2036963:C:T | rs1157558669 | ENST00000424542:ENST00000432365: |
| | | ENST00000563587:ENST00000566198 |
| 16:2036963:C:A | | ENST00000424542:ENST00000432365: |
| | | ENST00000563587:ENST00000566198 |
| 16:2036964:C:T | rs536133818 | ENST00000424542:ENST00000432365: |
| | | ENST00000563587:ENST00000566198 |
| 16:2036966:T:C | | ENST00000424542:ENST00000432365: |
| | | ENST00000563587:ENST00000566198 |
| 16:2036967:C:T | | ENST00000424542:ENST00000432365: |
| | | ENST00000563587:ENST00000566198 |
| 16:2036969:C:A | rs1161730214 | ENST00000424542:ENST00000432365: |
| | | ENST00000563587:ENST00000566198 |
| 16:2036970:C:T | | ENST00000424542:ENST00000432365: |
| | | ENST00000563587:ENST00000566198 |
| 16:2036972:G:A | rs57148397 | ENST00000424542:ENST00000432365: |
| | | ENST00000563587:ENST00000566198 |
| 16:2036973:T:C | | ENST00000424542:ENST00000432365: |
| | | ENST00000563587:ENST00000566198 |
| 16:2036975:A:G | rs767077612 | ENST00000424542:ENST00000432365: |
| | | ENST00000563587:ENST00000566198 |
| 16:2036976:C:A | rs1373872890 | ENST00000424542:ENST00000432365: |
| | | ENST00000563587:ENST00000566198 |
| 16:2036976:C:T | | ENST00000424542:ENST00000432365: |
| | | ENST00000563587:ENST00000566198 |
| 16:2036978:A:G | | ENST00000424542:ENST00000432365: |
| | | ENST00000563587:ENST00000566198 |
| 16:2036979:A:G | rs755523807 | ENST00000424542:ENST00000432365: |
| | | ENST00000563587:ENST00000566198 |
| 16:2036980:T:G | | ENST00000424542:ENST00000432365: |
| | | ENST00000563587:ENST00000566198 |
| 16:2036981:G:C | | ENST00000424542:ENST00000432365: |
| | | ENST00000563587:ENST00000566198 |
| 16:2036981:G:A | | ENST00000424542:ENST00000432365: |
| | | ENST00000563587:ENST00000566198 |
| 16:2036985:C:T | | ENST00000424542:ENST00000432365: |
| | | ENST00000563587:ENST00000566198 |
| 16:2036985:C:G | rs1225750410 | ENST00000424542:ENST00000432365: |
| | | ENST00000563587:ENST00000566198 |
| 16:2036988:G:C | rs114276628 | ENST00000424542:ENST00000432365: |
| | | ENST00000563587:ENST00000566198 |
| 16:2036988:G:A | | ENST00000424542:ENST00000432365: |
| | | ENST00000563587:ENST00000566198 |
| 16:2036990:C:T | | ENST00000424542:ENST00000432365: |
| | | ENST00000563587:ENST00000566198 |
| 16:2036993:G:A | rs371590353 | ENST00000424542:ENST00000432365: |
| | | ENST00000563587:ENST00000566198 |
| 16:2036993:G:T | | ENST00000424542:ENST00000432365: |
| | | ENST00000563587:ENST00000566198 |
| 16:2036994:CCCAGGTAAG:C | | ENST00000424542:ENST00000432365: |
| | | ENST00000563587:ENST00000566198 |
| 16:2036994:CCCAGG:C | | ENST00000424542:ENST00000432365: |
| | | ENST00000563587:ENST00000566198 |
| 16:2036997:A:G | | ENST00000424542:ENST00000432365: |
| | | ENST00000563587:ENST00000566198 |
| 16:2036999:G:A | rs1177408199 | ENST00000424542:ENST00000432365: |
| | | ENST00000563587:ENST00000566198 |
| 16:2037000:T:C | rs1278721257 | ENST00000424542:ENST00000432365: |
| | | ENST00000563587:ENST00000566198 |
| 16:2037536:A:C | | ENST00000424542:ENST00000432365: |
| | | ENST00000563587:ENST00000566198 |
| 16:2037538:C:T | | ENST00000424542:ENST00000432365: |
| | | ENST00000563587:ENST00000566198 |
| 16:2037542:A:T | rs369764975 | ENST00000424542:ENST00000432365: |
| | | ENST00000563587:ENST00000566198 |
| 16:2037542:A:G | rs369764975 | ENST00000424542:ENST00000432365: |
| | | ENST00000563587:ENST00000566198 |
| 16:2037544:G:A | | ENST00000424542:ENST00000432365: |
| | | ENST00000563587:ENST00000566198 |
| 16:2037544:G:T | | ENST00000424542:ENST00000432365: |
| | | ENST00000563587:ENST00000566198 |
| 16:2037545:G:C | | ENST00000424542:ENST00000432365: |
| | | ENST00000563587:ENST00000566198 |
| 16:2037547:G:A | rs751932265 | ENST00000424542:ENST00000432365: |
| | | ENST00000563587:ENST00000566198 |
| 16:2037548:G:A | rs757461813 | ENST00000424542:ENST00000432365: |
| | | ENST00000563587:ENST00000566198 |
| 16:2037554:C:G | | ENST00000424542:ENST00000432365: |
| | | ENST00000563587:ENST00000566198 |
| 16:2037554:C:T | rs781524063 | ENST00000424542:ENST00000432365: |
| | | ENST00000563587:ENST00000566198 |
| 16:2037556:T:C | rs879892972 | ENST00000424542:ENST00000432365: |
| | | ENST00000563587:ENST00000566198 |
| 16:2037557:G:A | | ENST00000424542:ENST00000432365: |
| | | ENST00000563587:ENST00000566198 |
| 16:2037560:C:T | rs878958184 | ENST00000424542:ENST00000432365: |
| | | ENST00000563587:ENST00000566198 |
| 16:2037562:T:C | rs761633456 | ENST00000424542:ENST00000432365: |
| | | ENST00000563587:ENST00000566198 |
| 16:2037565:C:T | | ENST00000424542:ENST00000432365: |
| | | ENST00000563587:ENST00000566198 |
| 16:2037565:C:G | rs772797226 | ENST00000424542:ENST00000432365: |
| | | ENST00000563587:ENST00000566198 |
| 16:2037566:G:A | rs376297831 | ENST00000424542:ENST00000432365: |
| | | ENST00000563587:ENST00000566198 |
| 16:2037568:A:G | | ENST00000424542:ENST00000432365: |
| | | ENST00000563587:ENST00000566198 |
| 16:2037571:G:C | rs1004438078 | ENST00000424542:ENST00000432365: |
| | | ENST00000563587:ENST00000566198 |
| 16:2037572:A:G | rs1259045644 | ENST00000424542:ENST00000432365: |
| | | ENST00000563587:ENST00000566198 |
| 16:2037577:C:A | | ENST00000424542:ENST00000432365: |
| | | ENST00000563587:ENST00000566198 |
| 16:2037578:C:T | rs1025321807 | ENST00000424542:ENST00000432365: |
| | | ENST00000563587:ENST00000566198 |
| 16:2037580:G:A | | ENST00000424542:ENST00000432365: |
| | | ENST00000563587:ENST00000566198 |
| 16:2037581:G:A | | ENST00000424542:ENST00000432365: |
| | | ENST00000563587:ENST00000566198 |
| 16:2037584:C:T | rs763419418 | ENST00000424542:ENST00000432365: |
| | | ENST00000563587:ENST00000566198 |
| 16:2037586:G:T | rs369206224 | ENST00000424542:ENST00000432365: |
| | | ENST00000563587:ENST00000566198 |
| 16:2037586:G:A | rs369206224 | ENST00000424542:ENST00000432365: |
| | | ENST00000563587:ENST00000566198 |
| 16:2037588:C:G | | ENST00000424542:ENST00000432365: |
| | | ENST00000563587:ENST00000566198 |
| 16:2037589:A:G | rs1195586589 | ENST00000424542:ENST00000432365: |
| | | ENST00000563587:ENST00000566198 |
| 16:2037589:A:C | | ENST00000424542:ENST00000432365: |
| | | ENST00000563587:ENST00000566198 |
| 16:2037592:G:C | | ENST00000424542:ENST00000432365: |
| | | ENST00000563587:ENST00000566198 |
| 16:2037592:G:A | rs1395860109 | ENST00000424542:ENST00000432365: |
| | | ENST00000563587:ENST00000566198 |
| 16:2037593:A:T | | ENST00000424542:ENST00000432365: |
| | | ENST00000563587:ENST00000566198 |
| 16:2037595:A:G | | ENST00000424542:ENST00000432365: |
| | | ENST00000563587:ENST00000566198 |
| 16:2037595:A:C | rs751900657 | ENST00000424542:ENST00000432365: |
| | | ENST00000563587:ENST00000566198 |
| 16:2037596:C:A | | ENST00000424542:ENST00000432365: |
| | | ENST00000563587:ENST00000566198 |
| 16:2037596:C:G | rs1156611443 | ENST00000424542:ENST00000432365: |
| | | ENST00000563587:ENST00000566198 |
| 16:2037601:G:A | rs980601266 | ENST00000424542:ENST00000432365: |
| | | ENST00000563587:ENST00000566198 |
| 16:2037830:G:A | | ENST00000424542:ENST00000563587:ENST00000566198 |
| 16:2037833:G:C | | ENST00000424542:ENST00000563587:ENST00000566198 |
| 16:2037835:G:C | | ENST00000424542:ENST00000563587:ENST00000566198 |
| 16:2037839:G:T | | ENST00000424542:ENST00000563587:ENST00000566198 |
| 16:2037841:A:T | | ENST00000424542:ENST00000563587:ENST00000566198 |
| 16:2037842:A:G | | ENST00000424542:ENST00000563587:ENST00000566198 |
| 16:2037845:A:G | rs985574755 | ENST00000424542:ENST00000563587:ENST00000566198 |
| 16:2037847:G:C | | ENST00000424542:ENST00000563587:ENST00000566198 |
| 16:2037848:A:C | | ENST00000424542:ENST00000563587:ENST00000566198 |
| 16:2037850:C:T | | ENST00000424542:ENST00000563587:ENST00000566198 |
| 16:2037851:C:T | | ENST00000424542:ENST00000563587:ENST00000566198 |
| 16:2037854:T:A | | ENST00000424542:ENST00000563587:ENST00000566198 |
| 16:2037860:A:G | | ENST00000424542:ENST00000432365: |
| | | ENST00000563587:ENST00000566198 |
| 16:2037860:A:C | | ENST00000424542:ENST00000432365: |
| | | ENST00000563587:ENST00000566198 |
| 16:2037861:G:C | rs761078183 | ENST00000424542:ENST00000432365: |
| | | ENST00000563587:ENST00000566198 |
| 16:2037863:G:T | | ENST00000424542:ENST00000432365: |
| | | ENST00000563587:ENST00000566198 |
| 16:2037863:G:A | rs766527921 | ENST00000424542:ENST00000432365: |
| | | ENST00000563587:ENST00000566198 |
| 16:2037864:C:G | rs201730988 | ENST00000424542:ENST00000432365: |
| | | ENST00000563587:ENST00000566198 |
| 16:2037864:C:A | rs201730988 | ENST00000424542:ENST00000432365: |
| | | ENST00000563587:ENST00000566198 |
| 16:2037865:G:C | | ENST00000424542:ENST00000432365: |
| | | ENST00000563587:ENST00000566198 |
| 16:2037865:G:A | rs765418448 | ENST00000424542:ENST00000432365: |
| | | ENST00000563587:ENST00000566198 |
| 16:2037866:G:A | rs752752331 | ENST00000424542:ENST00000432365: |
| | | ENST00000563587:ENST00000566198 |
| 16:2037875:T:C | | ENST00000424542:ENST00000432365: |
| | | ENST00000563587:ENST00000566198 |
| 16:2037884:C:T | rs377126399 | ENST00000424542:ENST00000432365: |
| | | ENST00000563587:ENST00000566198 |
| 16:2037884:C:A | | ENST00000424542:ENST00000432365: |
| | | ENST00000563587:ENST00000566198 |
| 16:2037887:C:G | rs201338478 | ENST00000424542:ENST00000432365: |
| | | ENST00000563587:ENST00000566198 |
| 16:2037887:C:T | rs201338478 | ENST00000424542:ENST00000432365: |
| | | ENST00000563587:ENST00000566198 |
| 16:2037892:G:A | rs371903014 | ENST00000424542:ENST00000432365: |
| | | ENST00000563587:ENST00000566198 |
| 16:2037892:G:C | | ENST00000424542:ENST00000432365: |
| | | ENST00000563587:ENST00000566198 |
| 16:2037894:G:C | rs1396454367 | ENST00000424542:ENST00000432365: |
| | | ENST00000563587:ENST00000566198 |
| 16:2037895:G:T | | ENST00000424542:ENST00000432365: |
| | | ENST00000563587:ENST00000566198 |
| 16:2037898:A:C | | ENST00000424542:ENST00000432365: |
| | | ENST00000563587:ENST00000566198 |
| 16:2037903:G:T | | ENST00000424542:ENST00000432365: |
| | | ENST00000563587:ENST00000566198 |
| 16:2037905:A:G | | ENST00000424542:ENST00000432365: |
| | | ENST00000563587:ENST00000566198 |
| 16:2037907:G:T | rs748774275 | ENST00000424542:ENST00000432365: |
| | | ENST00000563587:ENST00000566198 |
| 16:2037910:C:G | | ENST00000424542:ENST00000432365: |
| | | ENST00000563587:ENST00000566198 |
| 16:2037910:C:T | rs1174654735 | ENST00000424542:ENST00000432365: |
| | | ENST00000563587:ENST00000566198 |
| 16:2037911:G:C | | ENST00000424542:ENST00000432365: |
| | | ENST00000563587:ENST00000566198 |
| 16:2037911:G:A | rs772732824 | ENST00000424542:ENST00000432365: |
| | | ENST00000563587:ENST00000566198 |
| 16:2037913:G:A | | ENST00000424542:ENST00000432365: |
| | | ENST00000563587:ENST00000566198 |
| 16:2037914:C:A | | ENST00000424542:ENST00000432365: |
| | | ENST00000563587:ENST00000566198 |
| 16:2037917:T:C | | ENST00000424542:ENST00000432365: |
| | | ENST00000563587:ENST00000566198 |
| 16:2037917:T:A | | ENST00000424542:ENST00000432365: |
| | | ENST00000563587:ENST00000566198 |
| 16:2037919:C:G | | ENST00000424542:ENST00000432365: |
| | | ENST00000563587:ENST00000566198 |
| 16:2037919:C:T | rs1196318614 | ENST00000424542:ENST00000432365: |
| | | ENST00000563587:ENST00000566198 |
| 16:2037920:G:A | rs751467797 | ENST00000424542:ENST00000432365: |
| | | ENST00000563587:ENST00000566198 |
| 16:2037922:G:C | rs1363307485 | ENST00000424542:ENST00000432365: |
| | | ENST00000563587:ENST00000566198 |
| 16:2037925:A:G | rs776849615 | ENST00000424542:ENST00000432365: |
| | | ENST00000563587:ENST00000566198 |
| 16:2037926:A:G | rs759854877 | ENST00000424542:ENST00000432365: |
| | | ENST00000563587:ENST00000566198 |
| 16:2037927:C:G | | ENST00000424542:ENST00000432365: |
| | | ENST00000563587:ENST00000566198 |
| 16:2037927:C:A | | ENST00000424542:ENST00000432365: |
| | | ENST00000563587:ENST00000566198 |
| 16:2037929:A:G | | ENST00000424542:ENST00000432365: |
| | | ENST00000563587:ENST00000566198 |
| 16:2037931:C:G | | ENST00000424542:ENST00000432365: |
| | | ENST00000563587:ENST00000566198 |
| 16:2037931:C:T | rs1308227984 | ENST00000424542:ENST00000432365: |
| | | ENST00000563587:ENST00000566198 |
| 16:2037932:G:T | | ENST00000424542:ENST00000432365: |
| | | ENST00000563587:ENST00000566198 |
| 16:2037932:G:C | | ENST00000424542:ENST00000432365: |
| | | ENST00000563587:ENST00000566198 |
| 16:2037932:G:A | rs752805372 | ENST00000424542:ENST00000432365: |
| | | ENST00000563587:ENST00000566198 |
| 16:2037934:G:A | rs1261166515 | ENST00000424542:ENST00000432365: |
| | | ENST00000563587:ENST00000566198 |
| 16:2037935:C:T | rs1488127262 | ENST00000424542:ENST00000432365: |
| | | ENST00000563587:ENST00000566198 |
| 16:2037935:C:A | | ENST00000424542:ENST00000432365: |
| | | ENST00000563587:ENST00000566198 |
| 16:2037938:C:T | | ENST00000424542:ENST00000432365: |
| | | ENST00000563587:ENST00000566198 |
| 16:2037941:A:G | rs921611251 | ENST00000424542:ENST00000432365: |
| | | ENST00000563587:ENST00000566198 |
| 16:2037942:G:C | rs1237268733 | ENST00000424542:ENST00000432365: |
| | | ENST00000563587:ENST00000566198 |
| 16:2037944:T:C | | ENST00000424542:ENST00000432365: |
| | | ENST00000563587:ENST00000566198 |
| 16:2037947:A:G | | ENST00000424542:ENST00000432365: |
| | | ENST00000563587:ENST00000566198 |
| 16:2037947:A:T | | ENST00000424542:ENST00000432365: |
| | | ENST00000563587:ENST00000566198 |
| 16:2037948:C:G | rs1454425219 | ENST00000424542:ENST00000432365: |
| | | ENST00000563587:ENST00000566198 |
| 16:2037949:TG:T | | ENST00000424542:ENST00000432365: |
| | | ENST00000563587:ENST00000566198 |
| 16:2037949:T:A | | ENST00000424542:ENST00000432365: |
| | | ENST00000563587:ENST00000566198 |
| 16:2037949:T:TG | | ENST00000424542:ENST00000432365: |
| | | ENST00000563587:ENST00000566198 |
| 16:2037950:G:A | | ENST00000424542:ENST00000432365: |
| | | ENST00000563587:ENST00000566198 |
| 16:2037955:A:G | | ENST00000424542:ENST00000432365: |
| | | ENST00000563587:ENST00000566198 |
| 16:2037955:AG:A | | ENST00000424542:ENST00000432365: |
| | | ENST00000563587:ENST00000566198 |
| 16:2037956:G:C | rs1405374250 | ENST00000424542:ENST00000432365: |
| | | ENST00000563587:ENST00000566198 |
| 16:2037959:A:G | | ENST00000424542:ENST00000432365: |
| | | ENST00000563587:ENST00000566198 |
| 16:2037961:C:T | rs537470052 | ENST00000424542:ENST00000432365: |
| | | ENST00000563587:ENST00000566198 |
| 16:2037962:G:T | | ENST00000424542:ENST00000432365: |
| | | ENST00000563587:ENST00000566198 |
| 16:2037962:G:A | rs750159454 | ENST00000424542:ENST00000432365: |
| | | ENST00000563587:ENST00000566198 |
| 16:2037964:G:C | | ENST00000424542:ENST00000432365: |
| | | ENST00000563587:ENST00000566198 |
| 16:2037967:A:G | | ENST00000424542:ENST00000432365: |
| | | ENST00000563587:ENST00000566198 |
| 16:2037967:A:C | | ENST00000424542:ENST00000432365: |
| | | ENST00000563587:ENST00000566198 |
| 16:2037970:T:A | | ENST00000424542:ENST00000432365: |
| | | ENST00000563587:ENST00000566198 |
| 16:2037971:T:G | | ENST00000424542:ENST00000432365: |
| | | ENST00000563587:ENST00000566198 |
| 16:2037971:TCAGCA:T | | ENST00000424542:ENST00000432365: |
| | | ENST00000563587:ENST00000566198 |
| 16:2037974:G:A | | ENST00000424542:ENST00000432365: |
| | | ENST00000563587:ENST00000566198 |
| 16:2037977:AC:A | | ENST00000424542:ENST00000432365: |
| | | ENST00000563587:ENST00000566198 |
| 16:2037977:A:G | rs1157746210 | ENST00000424542:ENST00000432365: |
| | | ENST00000563587:ENST00000566198 |
| 16:2037978:CT:C | | ENST00000424542:ENST00000432365: |
| | | ENST00000563587:ENST00000566198 |
| 16:2037979:T:C | | ENST00000424542:ENST00000432365: |
| | | ENST00000563587:ENST00000566198 |
| 16:2037982:T:C | | ENST00000424542:ENST00000432365: |
| | | ENST00000563587:ENST00000566198 |
| 16:2037984:A:G | rs755828798 | ENST00000424542:ENST00000432365: |
| | | ENST00000563587:ENST00000566198 |

In some embodiments, the subject's aggregate burden of having any one or more SLC9A3R2 missense variant nucleic acid molecules encoding an SLC9A3R2 predicted loss-of-function polypeptide represents a weighted sum of a plurality of any of the SLC9A3R2 missense variant nucleic acid molecules encoding an SLC9A3R2 predicted loss-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the SLC9A3R2 gene where the genetic burden is the number of alleles multiplied by the association estimate with hypertension, coronary heart disease, and/or atrial fibrillation or related outcome for each allele (e.g., a weighted polygenic burden score). This can include any genetic variants, regardless of their genomic annotation, in proximity to the SLC9A3R2 gene (up to 10 Mb around the gene) that show a non-zero association with hypertension-related traits in a genetic association analysis. In some embodiments, when the subject has an aggregate burden above a desired threshold score, the subject has a decreased risk of developing hypertension, coronary heart disease, and/or atrial fibrillation. In some embodiments, when the subject has an aggregate burden below a desired threshold score, the subject has an increased risk of developing hypertension, coronary heart disease, and/or atrial fibrillation.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with hypertension, coronary heart disease, and/or atrial fibrillation in the top 10%, top 20%, top 30%, top 40%, or top 50% of p-value range for the association. In some embodiments, each of the identified genetic variants comprise the genetic variants having association with hypertension, coronary heart disease, and/or atrial fibrillation with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with hypertension, coronary heart disease, and/or atrial fibrillation with p-value of less than 5×10⁻⁸. In some embodiments, the identified genetic variants comprise genetic variants having association with hypertension, coronary heart disease, and/or atrial fibrillation in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing hypertension, coronary heart disease, and/or atrial fibrillation, the subject is further administered a therapeutic agent that treats or prevents hypertension, coronary heart disease, and/or atrial fibrillation, and/or an SLC9A3R2 inhibitor, as described herein. For example, when the subject is SLC9A3R2 reference, and therefore has an increased risk of developing hypertension, coronary heart disease, and/or atrial fibrillation, the subject is administered an SLC9A3R2 inhibitor. In some embodiments, such a subject is also administered a therapeutic agent that treats or prevents hypertension, coronary heart disease, and/or atrial fibrillation. In some embodiments, when the subject is heterozygous for an SLC9A3R2 missense variant nucleic acid molecule encoding an SLC9A3R2 predicted loss-of-function polypeptide, the subject is administered the therapeutic agent that treats or prevents hypertension, coronary heart disease, and/or atrial fibrillation in a dosage amount that is the same as or less than a standard dosage amount, and/or is administered an SLC9A3R2 inhibitor. In some embodiments, the subject is SLC9A3R2 reference. In some embodiments, the subject is heterozygous for an SLC9A3R2 missense variant nucleic acid molecule encoding an SLC9A3R2 predicted loss-of-function polypeptide. Furthermore, when the subject has a lower aggregate burden for having an SLC9A3R2 missense variant nucleic acid molecule encoding an SLC9A3R2 predicted loss-of-function polypeptide, and therefore has an increased risk of developing hypertension, coronary heart disease, and/or atrial fibrillation, the subject is administered a therapeutic agent that treats or prevents hypertension, coronary heart disease, and/or atrial fibrillation. In some embodiments, when the subject has a lower aggregate burden for having an SLC9A3R2 missense variant nucleic acid molecule encoding an SLC9A3R2 predicted loss-of-function polypeptide, the subject is administered the therapeutic agent that treats or prevents hypertension, coronary heart disease, and/or atrial fibrillation in a dosage amount that is the same as or greater than the standard dosage amount administered to a subject who has a greater aggregate burden for having an SLC9A3R2 missense variant nucleic acid molecule encoding an SLC9A3R2 predicted loss-of-function polypeptide.

The present disclosure also provides methods of detecting the presence or absence of an SLC9A3R2 missense variant genomic nucleic acid molecule encoding an SLC9A3R2 predicted loss-of-function polypeptide in a biological sample obtained from a subject, and/or an SLC9A3R2 missense variant mRNA molecule encoding an SLC9A3R2 predicted loss-of-function polypeptide in a biological sample obtained from a subject, and/or an SLC9A3R2 missense variant cDNA molecule encoding an SLC9A3R2 predicted loss-of-function polypeptide produced from an mRNA molecule in a biological sample obtained from a subject. It is understood that gene sequences within a population and mRNA molecules encoded by such genes can vary due to polymorphisms such as single nucleotide polymorphisms (SNPs). The sequences provided herein for the SLC9A3R2 variant genomic nucleic acid molecules, SLC9A3R2 variant mRNA molecules, and SLC9A3R2 variant cDNA molecules are only exemplary sequences. Other sequences for the SLC9A3R2 variant genomic nucleic acid molecules, variant mRNA molecules, and variant cDNA molecules are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any SLC9A3R2 variant nucleic acid molecule, preliminary processing designed to isolate or enrich the biological sample for the SLC9A3R2 variant nucleic acid molecule can be employed. A variety of techniques may be used for this purpose. When detecting the level of any SLC9A3R2 variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting an SLC9A3R2 missense variant nucleic acid molecule, or the complement thereof, encoding an SLC9A3R2 predicted loss-of-function polypeptide in a subject. The methods comprise assaying a biological sample obtained from the subject to determine whether a nucleic acid molecule in the biological sample is an SLC9A3R2 missense variant nucleic acid molecule encoding an SLC9A3R2 predicted loss-of-function polypeptide.

In some embodiments, the SLC9A3R2 missense variant nucleic acid molecule encoding the SLC9A3R2 predicted loss-of-function polypeptide, or the complement thereof, is a genomic nucleic acid molecule having a nucleotide sequence comprising a thymine at a position corresponding to position 9,519 according to SEQ ID NO:2, or the complement thereof.

In some embodiments, the SLC9A3R2 missense variant nucleic acid molecule encoding the SLC9A3R2 predicted loss-of-function polypeptide, or the complement thereof, is an mRNA molecule having a nucleotide sequence comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof.

In some embodiments, the SLC9A3R2 missense variant nucleic acid molecule encoding the SLC9A3R2 predicted loss-of-function polypeptide, or the complement thereof, is a cDNA molecule produced from an mRNA molecule in the biological sample having a nucleotide sequence comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

In some embodiments, the SLC9A3R2 missense variant nucleic acid molecule has a nucleotide sequence comprising: a thymine at a position corresponding to position 9,519 according to SEQ ID NO:2, or the complement thereof, (for genomic nucleic acid molecules); a uracil at a position corresponding to position 615 according to SEQ ID NO:22, or the complement thereof, (for mRNA molecules); or a thymine at a position corresponding to position 615 according to SEQ ID NO:59, or the complement thereof, (for cDNA molecules obtained from mRNA molecules).

In some embodiments, the biological sample comprises a cell or cell lysate. Such methods can further comprise, for example, obtaining a biological sample from the subject comprising an SLC9A3R2 genomic nucleic acid molecule or mRNA molecule, and if mRNA, optionally reverse transcribing the mRNA into cDNA. Such assays can comprise, for example determining the identity of these positions of the particular SLC9A3R2 nucleic acid molecule. In some embodiments, the method is an in vitro method.

In some embodiments, the assay comprises sequencing at least a portion of the nucleotide sequence of the SLC9A3R2 nucleic acid molecule, or the complement thereof, in the biological sample. In some embodiments, the assay comprises sequencing at least a portion of: the nucleotide sequence of the SLC9A3R2 genomic nucleic acid molecule in the biological sample, wherein the sequenced portion comprises a position corresponding to position 9,519 according to SEQ ID NO:2, or the complement thereof; the nucleotide sequence of the SLC9A3R2 mRNA molecule in the biological sample, wherein the sequenced portion comprises a position corresponding to position 615 according to SEQ ID NO:22, or the complement thereof; and/or the nucleotide sequence of the SLC9A3R2 cDNA molecule produced from the mRNA in the biological sample, wherein the sequenced portion comprises a position corresponding to position 615 according to SEQ ID NO:59, or the complement thereof. When the sequenced portion of the SLC9A3R2 nucleic acid molecule in the biological sample comprises: a thymine at a position corresponding to position 9,519 according to SEQ ID NO:2, or the complement thereof; a uracil at a position corresponding to position 615 according to SEQ ID NO:22, or the complement thereof; or a thymine at a position corresponding to position 615 according to SEQ ID NO:59, or the complement thereof; then the SLC9A3R2 nucleic acid molecule in the biological sample is an SLC9A3R2 missense variant nucleic acid molecule encoding an SLC9A3R2 predicted loss-of-function polypeptide.

In some embodiments, the assay comprises sequencing at least a portion of the nucleotide sequence of the SLC9A3R2 genomic nucleic acid molecule, or the complement thereof, in the biological sample, wherein the sequenced portion comprises a position corresponding to position 9,519 according to SEQ ID NO:2, or the complement thereof. When the sequenced portion of the SLC9A3R2 genomic nucleic acid molecule in the biological sample comprises: a thymine at a position corresponding to position 9,519 according to SEQ ID NO:2, or the complement thereof, then the SLC9A3R2 genomic nucleic acid molecule in the biological sample is an SLC9A3R2 missense variant genomic nucleic acid molecule encoding an SLC9A3R2 predicted loss-of-function polypeptide.

In some embodiments, the assay comprises sequencing at least a portion of the nucleotide sequence of the SLC9A3R2 mRNA molecule in the biological sample, wherein the sequenced portion comprises a position corresponding to: position 615 according to SEQ ID NO:22, or the complement thereof; position 589 according to SEQ ID NO:23, or the complement thereof; position 353 according to SEQ ID NO:24, or the complement thereof; position 230 according to SEQ ID NO:25, or the complement thereof; position 236 according to SEQ ID NO:26, or the complement thereof; position 236 according to SEQ ID NO:27, or the complement thereof; position 604 according to SEQ ID NO:28, or the complement thereof; or position 126 according to SEQ ID NO:29, or the complement thereof. When the sequenced portion of the SLC9A3R2 mRNA molecule in the biological sample comprises: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; then the SLC9A3R2 mRNA molecule in the biological sample is an SLC9A3R2 missense variant mRNA molecule encoding an SLC9A3R2 predicted loss-of-function polypeptide.

In some embodiments, the assay comprises sequencing at least a portion of the nucleotide sequence of the SLC9A3R2 cDNA molecule produced from an mRNA molecule in the biological sample, wherein the sequenced portion comprises a position corresponding to: position 615 according to SEQ ID NO:59, or the complement thereof; position 589 according to SEQ ID NO:60, or the complement thereof; position 353 according to SEQ ID NO:61, or the complement thereof; position 230 according to SEQ ID NO:62, or the complement thereof; position 236 according to SEQ ID NO:63, or the complement thereof; position 236 according to SEQ ID NO:64, or the complement thereof; position 604 according to SEQ ID NO:65, or the complement thereof; or position 126 according to SEQ ID NO:66, or the complement thereof. When the sequenced portion of the SLC9A3R2 cDNA molecule in the biological sample comprises: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof; then the SLC9A3R2 cDNA molecule produced from an mRNA molecule in the biological sample is an SLC9A3R2 missense variant cDNA molecule encoding an SLC9A3R2 predicted loss-of-function polypeptide.

In some embodiments, the assay comprises: a) contacting the biological sample with a primer hybridizing to a portion of the nucleotide sequence of the SLC9A3R2: genomic nucleic acid molecule, or the complement thereof, that is proximate to a position corresponding to position 9,519 according to SEQ ID NO:2, or the complement thereof; mRNA molecule, or the complement thereof, that is proximate to a position corresponding to position 615 according to SEQ ID NO:22, or the complement thereof; and/or cDNA molecule, or the complement thereof, that is proximate to a position corresponding to position 615 according to SEQ ID NO:59, or the complement thereof; b) extending the primer at least through the position of the nucleotide sequence of the SLC9A3R2: genomic nucleic acid molecule, or the complement thereof, corresponding to position 9,519 according to SEQ ID NO:2, or the complement thereof; mRNA molecule, or the complement thereof, corresponding to position 615 according to SEQ ID NO:22, or the complement thereof; and/or cDNA molecule, or the complement thereof, corresponding to position 615 according to SEQ ID NO:59, or the complement thereof; and c) determining whether the extension product of the primer comprises: a thymine at a position corresponding to position 9,519 according to SEQ ID NO:2, or the complement thereof; a uracil at a position corresponding to position 615 according to SEQ ID NO:22, or the complement thereof; and/or a thymine at a position corresponding to position 615 according to SEQ ID NO:59, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 589 according to SEQ ID NO:23, or the complement thereof; and/or cDNA molecule, or the complement thereof, that is proximate to a position corresponding to position 589 according to SEQ ID NO:60, or the complement thereof; b) extending the primer at least through the position of the nucleotide sequence of the SLC9A3R2: mRNA molecule, or the complement thereof, corresponding to position 589 according to SEQ ID NO:23, or the complement thereof; and/or cDNA molecule, or the complement thereof, corresponding to position 589 according to SEQ ID NO:60, or the complement thereof; and c) determining whether the extension product of the primer comprises: a uracil at a position corresponding to position 589 according to SEQ ID NO:23, or the complement thereof; and/or a thymine at a position corresponding to position 589 according to SEQ ID NO:60, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 353 according to SEQ ID NO:24, or the complement thereof; and/or cDNA molecule, or the complement thereof, that is proximate to a position corresponding to position 353 according to SEQ ID NO:61, or the complement thereof; b) extending the primer at least through the position of the nucleotide sequence of the SLC9A3R2: mRNA molecule, or the complement thereof, corresponding to position 353 according to SEQ ID NO:24, or the complement thereof; and/or cDNA molecule, or the complement thereof, corresponding to position 353 according to SEQ ID NO:61, or the complement thereof; and c) determining whether the extension product of the primer comprises: a uracil at a position corresponding to position 353 according to SEQ ID NO:24, or the complement thereof; and/or a thymine at a position corresponding to position 353 according to SEQ ID NO:61, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 230 according to SEQ ID NO:25, or the complement thereof; and/or cDNA molecule, or the complement thereof, that is proximate to a position corresponding to position 230 according to SEQ ID NO:62, or the complement thereof; b) extending the primer at least through the position of the nucleotide sequence of the SLC9A3R2: mRNA molecule, or the complement thereof, corresponding to position 230 according to SEQ ID NO:25, or the complement thereof; and/or cDNA molecule, or the complement thereof, corresponding to position 230 according to SEQ ID NO:62, or the complement thereof; and c) determining whether the extension product of the primer comprises: a uracil at a position corresponding to position 230 according to SEQ ID NO:25, or the complement thereof; and/or a thymine at a position corresponding to position 230 according to SEQ ID NO:62, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 236 according to SEQ ID NO:26, or the complement thereof; and/or cDNA molecule, or the complement thereof, that is proximate to a position corresponding to position 236 according to SEQ ID NO:63, or the complement thereof; b) extending the primer at least through the position of the nucleotide sequence of the SLC9A3R2: mRNA molecule, or the complement thereof, corresponding to position 236 according to SEQ ID NO:26, or the complement thereof; and/or cDNA molecule, or the complement thereof, corresponding to position 236 according to SEQ ID NO:63, or the complement thereof; and c) determining whether the extension product of the primer comprises: a uracil at a position corresponding to position 236 according to SEQ ID NO:26, or the complement thereof; and/or a thymine at a position corresponding to position 236 according to SEQ ID NO:63, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 236 according to SEQ ID NO:27, or the complement thereof; and/or cDNA molecule, or the complement thereof, that is proximate to a position corresponding to position 236 according to SEQ ID NO:64, or the complement thereof; b) extending the primer at least through the position of the nucleotide sequence of the SLC9A3R2: mRNA molecule, or the complement thereof, corresponding to position 236 according to SEQ ID NO:27, or the complement thereof; and/or cDNA molecule, or the complement thereof, corresponding to position 236 according to SEQ ID NO:64, or the complement thereof; and c) determining whether the extension product of the primer comprises: a uracil at a position corresponding to position 236 according to SEQ ID NO:27, or the complement thereof; and/or a thymine at a position corresponding to position 236 according to SEQ ID NO:64, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 604 according to SEQ ID NO:28, or the complement thereof; and/or cDNA molecule, or the complement thereof, that is proximate to a position corresponding to position 604 according to SEQ ID NO:65, or the complement thereof; b) extending the primer at least through the position of the nucleotide sequence of the SLC9A3R2: mRNA molecule, or the complement thereof, corresponding to position 604 according to SEQ ID NO:28, or the complement thereof; and/or cDNA molecule, or the complement thereof, corresponding to position 604 according to SEQ ID NO:65, or the complement thereof; and c) determining whether the extension product of the primer comprises: a uracil at a position corresponding to position 604 according to SEQ ID NO:28, or the complement thereof; and/or a thymine at a position corresponding to position 604 according to SEQ ID NO:65, or the complement thereof.

In some embodiments, the assay comprises: a) contacting the biological sample with a primer hybridizing to a portion of the nucleotide sequence of the SLC9A3R2: mRNA molecule, or the complement thereof, that is proximate to a position corresponding to position 126 according to SEQ ID NO:29, or the complement thereof; and/or cDNA molecule, or the complement thereof, that is proximate to a position corresponding to position 126 according to SEQ ID NO:66, or the complement thereof; b) extending the primer at least through the position of the nucleotide sequence of the SLC9A3R2: mRNA molecule, or the complement thereof, corresponding to position 126 according to SEQ ID NO:29, or the complement thereof; and/or cDNA molecule, or the complement thereof, corresponding to position 126 according to SEQ ID NO:66, or the complement thereof; and c) determining whether the extension product of the primer comprises: a uracil at a position corresponding to position 126 according to SEQ ID NO:29, or the complement thereof; and/or a thymine at a position corresponding to position 126 according to SEQ ID NO:66, or the complement thereof.

In some embodiments, the assay comprises: a) contacting the biological sample with a primer hybridizing to a portion of the nucleotide sequence of the SLC9A3R2 genomic nucleic acid molecule, or the complement thereof, that is proximate to a position corresponding to position 9,519 according to SEQ ID NO:2, or the complement thereof; b) extending the primer at least through the position of the nucleotide sequence of the SLC9A3R2 genomic nucleic acid molecule, or the complement thereof, corresponding to: position 9,519 according to SEQ ID NO:2, or the complement thereof; and c) determining whether the extension product of the primer comprises: a thymine at a position corresponding to position 9,519 according to SEQ ID NO:2.

In some embodiments, the assay comprises: a) contacting the biological sample with a primer hybridizing to a portion of the nucleotide sequence of the SLC9A3R2 mRNA molecule, or the complement thereof, that is proximate to a position corresponding to: position 615 according to SEQ ID NO:22, or the complement thereof; position 589 according to SEQ ID NO:23, or the complement thereof; position 353 according to SEQ ID NO:24, or the complement thereof; position 230 according to SEQ ID NO:25, or the complement thereof; position 236 according to SEQ ID NO:26, or the complement thereof; position 236 according to SEQ ID NO:27, or the complement thereof; position 604 according to SEQ ID NO:28, or the complement thereof; or position 126 according to SEQ ID NO:29, or the complement thereof; b) extending the primer at least through the position of the nucleotide sequence of the SLC9A3R2 mRNA molecule, or the complement thereof, corresponding to: position 615 according to SEQ ID NO:22, or the complement thereof; position 589 according to SEQ ID NO:23, or the complement thereof; position 353 according to SEQ ID NO:24, or the complement thereof; position 230 according to SEQ ID NO:25, or the complement thereof; position 236 according to SEQ ID NO:26, or the complement thereof; position 236 according to SEQ ID NO:27, or the complement thereof; position 604 according to SEQ ID NO:28, or the complement thereof; or position 126 according to SEQ ID NO:29, or the complement thereof; and c) determining whether the extension product of the primer comprises: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof.

In some embodiments, the assay comprises: a) contacting the biological sample with a primer hybridizing to a portion of the nucleotide sequence of the SLC9A3R2 cDNA molecule, or the complement thereof, that is proximate to a position corresponding to: position 615 according to SEQ ID NO:59, or the complement thereof; position 589 according to SEQ ID NO:60, or the complement thereof; position 353 according to SEQ ID NO:61, or the complement thereof; position 230 according to SEQ ID NO:62, or the complement thereof; position 236 according to SEQ ID NO:63, or the complement thereof; position 236 according to SEQ ID NO:64, or the complement thereof; position 604 according to SEQ ID NO:65, or the complement thereof; or position 126 according to SEQ ID NO:66, or the complement thereof; b) extending the primer at least through the position of the nucleotide sequence of the SLC9A3R2 cDNA molecule, or the complement thereof, corresponding to: position 615 according to SEQ ID NO:59, or the complement thereof; position 589 according to SEQ ID NO:60, or the complement thereof; position 353 according to SEQ ID NO:61, or the complement thereof; position 230 according to SEQ ID NO:62, or the complement thereof; position 236 according to SEQ ID NO:63, or the complement thereof; position 236 according to SEQ ID NO:64, or the complement thereof; position 604 according to SEQ ID NO:65, or the complement thereof; or position 126 according to SEQ ID NO:66, or the complement thereof; and c) determining whether the extension product of the primer comprises: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

In some embodiments, the assay comprises sequencing the entire nucleic acid molecule. In some embodiments, only an SLC9A3R2 genomic nucleic acid molecule is analyzed. In some embodiments, only an SLC9A3R2 mRNA is analyzed. In some embodiments, only an SLC9A3R2 cDNA obtained from an SLC9A3R2 mRNA is analyzed.

In some embodiments, the assay comprises: a) amplifying at least a portion of the SLC9A3R2 nucleic acid molecule, or the complement thereof, in the biological sample, wherein the amplified portion comprises: a thymine at a position corresponding to position 9,519 according to SEQ ID NO:2, or the complement thereof; a uracil at a position corresponding to position 615 according to SEQ ID NO:22, or the complement thereof; and/or a thymine at a position corresponding to position 615 according to SEQ ID NO:59, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a thymine at a position corresponding to position 9,519 according to SEQ ID NO:2, or the complement thereof; a uracil at a position corresponding to position 615 according to SEQ ID NO:22, or the complement thereof; and/or a thymine at a position corresponding to position 615 according to SEQ ID NO:59, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 589 according to SEQ ID NO:23, or the complement thereof; and/or a thymine at a position corresponding to position 589 according to SEQ ID NO:60, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 589 according to SEQ ID NO:23, or the complement thereof; and/or a thymine at a position corresponding to position 589 according to SEQ ID NO:60, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 353 according to SEQ ID NO:24, or the complement thereof; and/or a thymine at a position corresponding to position 353 according to SEQ ID NO:61, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 353 according to SEQ ID NO:24, or the complement thereof; and/or a thymine at a position corresponding to position 353 according to SEQ ID NO:61, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 230 according to SEQ ID NO:25, or the complement thereof; and/or a thymine at a position corresponding to position 230 according to SEQ ID NO:62, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 230 according to SEQ ID NO:25, or the complement thereof; and/or a thymine at a position corresponding to position 230 according to SEQ ID NO:62, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 236 according to SEQ ID NO:26, or the complement thereof; and/or a thymine at a position corresponding to position 236 according to SEQ ID NO:63, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 236 according to SEQ ID NO:26, or the complement thereof; and/or a thymine at a position corresponding to position 236 according to SEQ ID NO:63, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 236 according to SEQ ID NO:27, or the complement thereof; and/or a thymine at a position corresponding to position 236 according to SEQ ID NO:64, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 236 according to SEQ ID NO:27, or the complement thereof; and/or a thymine at a position corresponding to position 236 according to SEQ ID NO:64, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 604 according to SEQ ID NO:28, or the complement thereof; and/or a thymine at a position corresponding to position 604 according to SEQ ID NO:65, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 604 according to SEQ ID NO:28, or the complement thereof; and/or a thymine at a position corresponding to position 604 according to SEQ ID NO:65, or the complement thereof; and d) detecting the detectable label.

In some embodiments, the assay comprises: a) amplifying at least a portion of the SLC9A3R2 nucleic acid molecule, or the complement thereof, in the biological sample, wherein the amplified portion comprises: a uracil at a position corresponding to position 126 according to SEQ ID NO:29, or the complement thereof; and/or a thymine at a position corresponding to position 126 according to SEQ ID NO:66, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 126 according to SEQ ID NO:29, or the complement thereof; and/or a thymine at a position corresponding to position 126 according to SEQ ID NO:66, or the complement thereof; and d) detecting the detectable label.

In some embodiments, the assay comprises: a) amplifying at least a portion of the SLC9A3R2 genomic nucleic acid molecule, or the complement thereof, in the biological sample, wherein the portion comprises: a thymine at a position corresponding to position 9,519 according to SEQ ID NO:2, or the complement thereof; and d) detecting the detectable label.

In some embodiments, the assay comprises: a) amplifying at least a portion of the SLC9A3R2 mRNA molecule, or the complement thereof, in the biological sample, wherein the portion comprises: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; and d) detecting the detectable label.

In some embodiments, the assay comprises: a) amplifying at least a portion of the SLC9A3R2 cDNA molecule, or the complement thereof, produced from an mRNA molecule in the biological sample, wherein the portion comprises: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof; and d) detecting the detectable label.

In some embodiments, the nucleic acid molecule in the sample is mRNA and the mRNA is reverse-transcribed into cDNA prior to the amplifying step.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a thymine at a position corresponding to position 9,519 according to SEQ ID NO:2, or the complement thereof; a uracil at a position corresponding to position 615 according to SEQ ID NO:22, or the complement thereof; and/or a thymine at a position corresponding to position 615 according to SEQ ID NO:59, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 589 according to SEQ ID NO:23, or the complement thereof; and/or a thymine at a position corresponding to position 589 according to SEQ ID NO:60, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 353 according to SEQ ID NO:24, or the complement thereof; and/or a thymine at a position corresponding to position 353 according to SEQ ID NO:61, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 230 according to SEQ ID NO:25, or the complement thereof; and/or a thymine at a position corresponding to position 230 according to SEQ ID NO:62, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 236 according to SEQ ID NO:26, or the complement thereof; and/or a thymine at a position corresponding to position 236 according to SEQ ID NO:63, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 236 according to SEQ ID NO:27, or the complement thereof; and/or a thymine at a position corresponding to position 236 according to SEQ ID NO:64, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 604 according to SEQ ID NO:28, or the complement thereof; and/or a thymine at a position corresponding to position 604 according to SEQ ID NO:65, or the complement thereof; and detecting the detectable label.

In some embodiments, the assay comprises: contacting the SLC9A3R2 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 nucleic acid molecule, or the complement thereof, comprising: a uracil at a position corresponding to position 126 according to SEQ ID NO:29, or the complement thereof; and/or a thymine at a position corresponding to position 126 according to SEQ ID NO:66, or the complement thereof; and detecting the detectable label.

In some embodiments, the assay comprises: contacting the SLC9A3R2 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 genomic nucleic acid molecule, or the complement thereof, comprising: a thymine at a position corresponding to position 9,519 according to SEQ ID NO:2, or the complement thereof; and detecting the detectable label.

In some embodiments, the assay comprises: contacting the SLC9A3R2 mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the SLC9A3R2 mRNA molecule, or the complement thereof, comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; and detecting the detectable label.

In some embodiments, the assay comprises: contacting the SLC9A3R2 cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the SLC9A3R2 cDNA molecule, or the complement thereof, comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof; and detecting the detectable label.

In some embodiments, the SLC9A3R2 nucleic acid molecule is present within a cell obtained from the subject.

Alteration-specific polymerase chain reaction techniques can be used to detect mutations such as SNPs in a nucleotide sequence. Alteration-specific primers can be used because the DNA polymerase will not extend when a mismatch with the template is present.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an SLC9A3R2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an SLC9A3R2 nucleic acid molecule (genomic nucleic acid molecule, mRNA molecule, or cDNA molecule), or complement thereof, within a biological sample comprises a nucleotide sequence comprising a thymine at a position corresponding to position 9,519 according to SEQ ID NO:2 (genomic nucleic acid molecule), a uracil at a position corresponding to position 615 according to SEQ ID NO:22 (mRNA molecule), or a thymine at a position corresponding to position 615 according to SEQ ID NO:59 (cDNA molecule), the biological sample can be subjected to an amplification method using a primer pair that includes a first primer derived from the 5′ flanking sequence adjacent to a thymine at a position corresponding to position 9,519 according to SEQ ID NO:2, a uracil at a position corresponding to position 615 according to SEQ ID NO:22, or a thymine at a position corresponding to position 615 according to SEQ ID NO:59, and a second primer derived from the 3′ flanking sequence adjacent to a thymine at a position corresponding to position 9,519 according to SEQ ID NO:2, a uracil at a position corresponding to position 615 according to SEQ ID NO:22, or a thymine at a position corresponding to position 615 according to SEQ ID NO:59 to produce an amplicon that is indicative of the presence of the SNP at positions encoding a thymine at a position corresponding to position 9,519 according to SEQ ID NO:2, a uracil at a position corresponding to position 615 according to SEQ ID NO:22, or a thymine at a position corresponding to position 615 according to SEQ ID NO:59. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 9,519 according to SEQ ID NO:2, a uracil at a position corresponding to position 615 according to SEQ ID NO:22, or a thymine at a position corresponding to position 615 according to SEQ ID NO:59, and at least 1, 2, 3, 4, 5, 6, 7, 8, 9, 10, or more nucleotides on each side of positions comprising a thymine at a position corresponding to position 9,519 according to SEQ ID NO:2, a uracil at a position corresponding to position 615 according to SEQ ID NO:22, or a thymine at a position corresponding to position 615 according to SEQ ID NO:59.

In some embodiments, to determine whether an SLC9A3R2 nucleic acid molecule (genomic nucleic acid molecule, mRNA molecule, or cDNA molecule), or complement thereof, within a biological sample comprises a nucleotide sequence comprising, a uracil at a position corresponding to position 589 according to SEQ ID NO:23 (mRNA molecule), or a thymine at a position corresponding to position 589 according to SEQ ID NO:60 (cDNA molecule), the biological sample can be subjected to an amplification method using a primer pair that includes a first primer derived from the 5′ flanking sequence adjacent to a uracil at a position corresponding to position 589 according to SEQ ID NO:23, or a thymine at a position corresponding to position 589 according to SEQ ID NO:60, and a second primer derived from the 3′ flanking sequence adjacent to a uracil at a position corresponding to position 589 according to SEQ ID NO:23, or a thymine at a position corresponding to position 589 according to SEQ ID NO:60 to produce an amplicon that is indicative of the presence of the SNP at positions encoding a uracil at a position corresponding to position 589 according to SEQ ID NO:23, or a thymine at a position corresponding to position 589 according to SEQ ID NO:60. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 589 according to SEQ ID NO:23, or a thymine at a position corresponding to position 589 according to SEQ ID NO:60, and at least 1, 2, 3, 4, 5, 6, 7, 8, 9, 10, or more nucleotides on each side of positions comprising a uracil at a position corresponding to position 589 according to SEQ ID NO:23, or a thymine at a position corresponding to position 589 according to SEQ ID NO:60.

In some embodiments, to determine whether an SLC9A3R2 nucleic acid molecule (genomic nucleic acid molecule, mRNA molecule, or cDNA molecule), or complement thereof, within a biological sample comprises a nucleotide sequence comprising a uracil at a position corresponding to position 353 according to SEQ ID NO:24 (mRNA molecule), or a thymine at a position corresponding to position 353 according to SEQ ID NO:61 (cDNA molecule), the biological sample can be subjected to an amplification method using a primer pair that includes a first primer derived from the 5′ flanking sequence adjacent to a uracil at a position corresponding to position 353 according to SEQ ID NO:24, or a thymine at a position corresponding to position 353 according to SEQ ID NO:61, and a second primer derived from the 3′ flanking sequence adjacent to a uracil at a position corresponding to position 353 according to SEQ ID NO:24, or a thymine at a position corresponding to position 353 according to SEQ ID NO:61 to produce an amplicon that is indicative of the presence of the SNP at positions encoding a uracil at a position corresponding to position 353 according to SEQ ID NO:24, or a thymine at a position corresponding to position 353 according to SEQ ID NO:61. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 353 according to SEQ ID NO:24, or a thymine at a position corresponding to position 353 according to SEQ ID NO:61, and at least 1, 2, 3, 4, 5, 6, 7, 8, 9, 10, or more nucleotides on each side of positions comprising a uracil at a position corresponding to position 353 according to SEQ ID NO:24, or a thymine at a position corresponding to position 353 according to SEQ ID NO:61.

In some embodiments, to determine whether an SLC9A3R2 nucleic acid molecule (genomic nucleic acid molecule, mRNA molecule, or cDNA molecule), or complement thereof, within a biological sample comprises a nucleotide sequence comprising a uracil at a position corresponding to position 230 according to SEQ ID NO:25 (mRNA molecule), or a thymine at a position corresponding to position 230 according to SEQ ID NO:62 (cDNA molecule), the biological sample can be subjected to an amplification method using a primer pair that includes a first primer derived from the 5′ flanking sequence adjacent to a uracil at a position corresponding to position 230 according to SEQ ID NO:25, or a thymine at a position corresponding to position 230 according to SEQ ID NO:62, and a second primer derived from the 3′ flanking sequence adjacent to a uracil at a position corresponding to position 230 according to SEQ ID NO:25, or a thymine at a position corresponding to position 230 according to SEQ ID NO:62 to produce an amplicon that is indicative of the presence of the SNP at positions encoding a uracil at a position corresponding to position 230 according to SEQ ID NO:25, or a thymine at a position corresponding to position 230 according to SEQ ID NO:62. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 230 according to SEQ ID NO:25, or a thymine at a position corresponding to position 230 according to SEQ ID NO:62, and at least 1, 2, 3, 4, 5, 6, 7, 8, 9, 10, or more nucleotides on each side of positions comprising a uracil at a position corresponding to position 230 according to SEQ ID NO:25, or a thymine at a position corresponding to position 230 according to SEQ ID NO:62.

In some embodiments, to determine whether an SLC9A3R2 nucleic acid molecule (genomic nucleic acid molecule, mRNA molecule, or cDNA molecule), or complement thereof, within a biological sample comprises a nucleotide sequence, a uracil at a position corresponding to position 236 according to SEQ ID NO:26 (mRNA molecule), or a thymine at a position corresponding to position 236 according to SEQ ID NO:63 (cDNA molecule), the biological sample can be subjected to an amplification method using a primer pair that includes a first primer derived from the 5′ flanking sequence adjacent to a uracil at a position corresponding to position 236 according to SEQ ID NO:26, or a thymine at a position corresponding to position 236 according to SEQ ID NO:63, and a second primer derived from the 3′ flanking sequence adjacent to a uracil at a position corresponding to position 236 according to SEQ ID NO:26, or a thymine at a position corresponding to position 236 according to SEQ ID NO:63 to produce an amplicon that is indicative of the presence of the SNP at positions encoding a uracil at a position corresponding to position 236 according to SEQ ID NO:26, or a thymine at a position corresponding to position 236 according to SEQ ID NO:63. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 236 according to SEQ ID NO:26, or a thymine at a position corresponding to position 236 according to SEQ ID NO:63, and at least 1, 2, 3, 4, 5, 6, 7, 8, 9, 10, or more nucleotides on each side of positions comprising a uracil at a position corresponding to position 236 according to SEQ ID NO:26, or a thymine at a position corresponding to position 236 according to SEQ ID NO:63.

In some embodiments, to determine whether an SLC9A3R2 nucleic acid molecule (genomic nucleic acid molecule, mRNA molecule, or cDNA molecule), or complement thereof, within a biological sample comprises a nucleotide sequence comprising, a uracil at a position corresponding to position 236 according to SEQ ID NO:27 (mRNA molecule), or a thymine at a position corresponding to position 236 according to SEQ ID NO:64 (cDNA molecule), the biological sample can be subjected to an amplification method using a primer pair that includes a first primer derived from the 5′ flanking sequence adjacent to a uracil at a position corresponding to position 236 according to SEQ ID NO:27, or a thymine at a position corresponding to position 236 according to SEQ ID NO:64, and a second primer derived from the 3′ flanking sequence adjacent to a uracil at a position corresponding to position 236 according to SEQ ID NO:27, or a thymine at a position corresponding to position 236 according to SEQ ID NO:64 to produce an amplicon that is indicative of the presence of the SNP at positions encoding a uracil at a position corresponding to position 236 according to SEQ ID NO:27, or a thymine at a position corresponding to position 236 according to SEQ ID NO:64. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 236 according to SEQ ID NO:27, or a thymine at a position corresponding to position 236 according to SEQ ID NO:64, and at least 1, 2, 3, 4, 5, 6, 7, 8, 9, 10, or more nucleotides on each side of positions comprising a uracil at a position corresponding to position 236 according to SEQ ID NO:27, or a thymine at a position corresponding to position 236 according to SEQ ID NO:64.

In some embodiments, to determine whether an SLC9A3R2 nucleic acid molecule (genomic nucleic acid molecule, mRNA molecule, or cDNA molecule), or complement thereof, within a biological sample comprises a nucleotide sequence a uracil at a position corresponding to position 604 according to SEQ ID NO:28 (mRNA molecule), or a thymine at a position corresponding to position 604 according to SEQ ID NO:65 (cDNA molecule), the biological sample can be subjected to an amplification method using a primer pair that includes a first primer derived from the 5′ flanking sequence adjacent to a uracil at a position corresponding to position 604 according to SEQ ID NO:28, or a thymine at a position corresponding to position 604 according to SEQ ID NO:65, and a second primer derived from the 3′ flanking sequence adjacent to a uracil at a position corresponding to position 604 according to SEQ ID NO:28, or a thymine at a position corresponding to position 604 according to SEQ ID NO:65 to produce an amplicon that is indicative of the presence of the SNP at positions encoding a uracil at a position corresponding to position 604 according to SEQ ID NO:28, or a thymine at a position corresponding to position 604 according to SEQ ID NO:65. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 604 according to SEQ ID NO:28, or a thymine at a position corresponding to position 604 according to SEQ ID NO:65, and at least 1, 2, 3, 4, 5, 6, 7, 8, 9, 10, or more nucleotides on each side of positions comprising a uracil at a position corresponding to position 604 according to SEQ ID NO:28, or a thymine at a position corresponding to position 604 according to SEQ ID NO:65.

In some embodiments, to determine whether an SLC9A3R2 nucleic acid molecule (genomic nucleic acid molecule, mRNA molecule, or cDNA molecule), or complement thereof, within a biological sample comprises a nucleotide sequence comprising, a uracil at a position corresponding to position 126 according to SEQ ID NO:29 (mRNA molecule), or a thymine at a position corresponding to position 126 according to SEQ ID NO:66 (cDNA molecule), the biological sample can be subjected to an amplification method using a primer pair that includes a first primer derived from the 5′ flanking sequence adjacent to a uracil at a position corresponding to position 126 according to SEQ ID NO:29, or a thymine at a position corresponding to position 126 according to SEQ ID NO:66, and a second primer derived from the 3′ flanking sequence adjacent to a uracil at a position corresponding to position 126 according to SEQ ID NO:29, or a thymine at a position corresponding to position 126 according to SEQ ID NO:66 to produce an amplicon that is indicative of the presence of the SNP at positions encoding a uracil at a position corresponding to position 126 according to SEQ ID NO:29, or a thymine at a position corresponding to position 126 according to SEQ ID NO:66. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 126 according to SEQ ID NO:29, or a thymine at a position corresponding to position 126 according to SEQ ID NO:66, and at least 1, 2, 3, 4, 5, 6, 7, 8, 9, 10, or more nucleotides on each side of positions comprising a uracil at a position corresponding to position 126 according to SEQ ID NO:29, or a thymine at a position corresponding to position 126 according to SEQ ID NO:66.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of an SLC9A3R2 predicted loss-of-function polypeptide comprising performing an assay on a biological sample obtained from the subject to determine whether an SLC9A3R2 polypeptide in the biological sample contains one or more variations that causes the polypeptide to have a loss-of-function (partial or complete) or predicted loss-of-function (partial or complete). The SLC9A3R2 predicted loss-of-function polypeptide can be any of the SLC9A3R2 predicted loss-of-function polypeptide described herein. In some embodiments, the methods detect the presence of SLC9A3R2 Arg171Trp-Long, Arg171Trp-Short, Arg65Trp, Arg58Trp, Arg60Trp-Short, Arg60Trp-Long, or Arg170Trp. In some embodiments, the methods detect the presence of SLC9A3R2 Arg171Trp-Long or Arg171Trp-Short.

In some embodiments, the methods comprise performing an assay on a biological sample obtained from a subject to determine whether an SLC9A3R2 polypeptide in the biological sample comprises: tryptophan at a position corresponding to position 171 according to SEQ ID NO:86; tryptophan at a position corresponding to position 171 according to SEQ ID NO:87; tryptophan at a position corresponding to position 65 according to SEQ ID NO:88; tryptophan at a position corresponding to position 58 according to SEQ ID NO:89; tryptophan at a position corresponding to position 60 according to SEQ ID NO:90; tryptophan at a position corresponding to position 60 according to SEQ ID NO:91; or tryptophan at a position corresponding to position 60 according to SEQ ID NO:92.

In some embodiments, the assay comprises sequencing at least a portion of the SLC9A3R2 polypeptide that comprises a position corresponding to: position 171 according to SEQ ID NO:86 or SEQ ID NO:77; position 171 according to SEQ ID NO:87 or SEQ ID NO:78; position 65 according to SEQ ID NO:88 or SEQ ID NO:79; position 58 according to SEQ ID NO:89 or SEQ ID NO:80; position 60 according to SEQ ID NO:90 or SEQ ID NO:81; position 60 according to SEQ ID NO:91 or SEQ ID NO:82; or position 60 according to SEQ ID NO:92 or SEQ ID NO:83.

In some embodiments, the assay is an immunoassay for detecting the presence of a SLC9A3R2 polypeptide that comprises a position corresponding to: position 171 according to SEQ ID NO:86 or SEQ ID NO:77; position 171 according to SEQ ID NO:87 or SEQ ID NO:78; position 65 according to SEQ ID NO:88 or SEQ ID NO:79; position 58 according to SEQ ID NO:89 or SEQ ID NO:80; position 60 according to SEQ ID NO:90 or SEQ ID NO:81; position 60 according to SEQ ID NO:91 or SEQ ID NO:82; or position 60 according to SEQ ID NO:92 or SEQ ID NO:83.

In some embodiments, when the subject does not have an SLC9A3R2 predicted loss-of-function polypeptide, the subject has an increased risk of developing hypertension, coronary heart disease, and/or atrial fibrillation, or any of primary hypertension, secondary hypertension, resistant hypertension, or malignant hypertension. In some embodiments, when the subject has an SLC9A3R2 predicted loss-of-function polypeptide, the subject has a decreased risk of developing hypertension, coronary heart disease, and/or atrial fibrillation, or any of primary hypertension, secondary hypertension, resistant hypertension, or malignant hypertension.

The present disclosure also provides isolated nucleic acid molecules that hybridize to SLC9A3R2 missense variant genomic nucleic acid molecules, SLC9A3R2 missense variant mRNA molecules, and/or SLC9A3R2 missense variant cDNA molecules (such as any of the genomic missense variant nucleic acid molecules, mRNA missense variant molecules, and cDNA missense variant molecules disclosed herein). In some embodiments, such isolated nucleic acid molecules hybridize to SLC9A3R2 missense variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the SLC9A3R2 missense nucleic acid molecule that includes a position corresponding to: position 9,519 according to SEQ ID NO:2, position 615 according to SEQ ID NO:22, or position 615 according to SEQ ID NO:59. In some embodiments, the isolated nucleic acid molecules hybridize to a portion of the SLC9A3R2 missense nucleic acid molecule that includes a position corresponding to: position 589 according to SEQ ID NO:23, or position 589 according to SEQ ID NO:60. In some embodiments, the isolated nucleic acid molecules hybridize to a portion of the SLC9A3R2 missense nucleic acid molecule that includes a position corresponding to: position 353 according to SEQ ID NO:24, or position 353 according to SEQ ID NO:61. In some embodiments, the isolated nucleic acid molecules hybridize to a portion of the SLC9A3R2 missense nucleic acid molecule that includes a position corresponding to: position 230 according to SEQ ID NO:25, or position 230 according to SEQ ID NO:62. In some embodiments, the isolated nucleic acid molecules hybridize to a portion of the SLC9A3R2 missense nucleic acid molecule that includes a position corresponding to: position 236 according to SEQ ID NO:26, or position 236 according to SEQ ID NO:63. In some embodiments, the isolated nucleic acid molecules hybridize to a portion of the SLC9A3R2 missense nucleic acid molecule that includes a position corresponding to: position 236 according to SEQ ID NO:27, or position 236 according to SEQ ID NO:64. In some embodiments, the isolated nucleic acid molecules hybridize to a portion of the SLC9A3R2 missense nucleic acid molecule that includes a position corresponding to: position 604 according to SEQ ID NO:28, or position 604 according to SEQ ID NO:65. In some embodiments, the isolated nucleic acid molecules hybridize to a portion of the SLC9A3R2 missense nucleic acid molecule that includes a position corresponding to: position 126 according to SEQ ID NO:29, or position 126 according to SEQ ID NO:66.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of a Solute Carrier Family 9 Isoform A3 Regulatory Factor 2 missense nucleic acid molecule encoding a SLC9A3R2 predicted loss-of-function polypeptide, or the complement thereof. In some embodiments, the portion comprises a position corresponding to: position 9,519 according to SEQ ID NO:2, or the complement thereof; position 615 according to SEQ ID NO:22, or the complement thereof; or position 615 according to SEQ ID NO:59, or the complement thereof. In some embodiments, the portion comprises a position corresponding to: position 589 according to SEQ ID NO:23, or the complement thereof; or position 589 according to SEQ ID NO:60, or the complement thereof. In some embodiments, the portion comprises a position corresponding to: position 353 according to SEQ ID NO:24, or the complement thereof; or position 353 according to SEQ ID NO:61, or the complement thereof. In some embodiments, the portion comprises a position corresponding to: position 230 according to SEQ ID NO:25, or the complement thereof; or position 230 according to SEQ ID NO:62, or the complement thereof. In some embodiments, the portion comprises a position corresponding to: position 236 according to SEQ ID NO:26, or the complement thereof; or position 236 according to SEQ ID NO:63, or the complement thereof. In some embodiments, the portion comprises a position corresponding to: position 236 according to SEQ ID NO:27, or the complement thereof; or position 236 according to SEQ ID NO:64, or the complement thereof. In some embodiments, the portion comprises a position corresponding to: position 604 according to SEQ ID NO:28, or the complement thereof; or position 604 according to SEQ ID NO:65, or the complement thereof. In some embodiments, the portion comprises a position corresponding to: position 126 according to SEQ ID NO:29, or the complement thereof; or position 126 according to SEQ ID NO:66, or the complement thereof.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to SLC9A3R2 missense variant genomic nucleic acid molecules, SLC9A3R2 missense variant mRNA molecules, and/or SLC9A3R2 missense variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 9,519 according to SEQ ID NO:2, or the complement thereof; position 615 according to SEQ ID NO:22, or the complement thereof; or position 615 according to SEQ ID NO:59, or the complement thereof. In some embodiments, the portion comprises positions corresponding to: positions 9,519-9,521 according to SEQ ID NO:2, or the complement thereof; positions 615-617 according to SEQ ID NO:22, or the complement thereof; and/or positions 615-617 according to SEQ ID NO:59, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 589 according to SEQ ID NO:23, or the complement thereof; or position 589 according to SEQ ID NO:60, or the complement thereof. In some embodiments, the portion comprises positions corresponding to: positions 589-591 according to SEQ ID NO:23, or the complement thereof; and/or positions 589-591 according to SEQ ID NO:60, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 353 according to SEQ ID NO:24, or the complement thereof; or position 353 according to SEQ ID NO:61, or the complement thereof. In some embodiments, the portion comprises positions corresponding to: positions 353-355 according to SEQ ID NO:24, or the complement thereof; and/or positions 353-355 according to SEQ ID NO:61, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 230 according to SEQ ID NO:25, or the complement thereof; or position 230 according to SEQ ID NO:62, or the complement thereof. In some embodiments, the portion comprises positions corresponding to: positions 230-232 according to SEQ ID NO:25, or the complement thereof; and/or positions 230-232 according to SEQ ID NO:62, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 236 according to SEQ ID NO:26, or the complement thereof; or position 236 according to SEQ ID NO:63, or the complement thereof. In some embodiments, the portion comprises positions corresponding to: positions 236-238 according to SEQ ID NO:26, or the complement thereof; and/or positions 236-238 according to SEQ ID NO:63, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 236 according to SEQ ID NO:27, or the complement thereof; or position 236 according to SEQ ID NO:64, or the complement thereof. In some embodiments, the portion comprises positions corresponding to: positions 236-238 according to SEQ ID NO:27, or the complement thereof; and/or positions 236-238 according to SEQ ID NO:64, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 604 according to SEQ ID NO:28, or the complement thereof; or position 604 according to SEQ ID NO:65, or the complement thereof. In some embodiments, the portion comprises positions corresponding to: positions 604-606 according to SEQ ID NO:28, or the complement thereof; and/or positions 604-606 according to SEQ ID NO:65, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an SLC9A3R2 missense variant nucleic acid molecule encoding an SLC9A3R2 predicted loss-of-function polypeptide, wherein the portion comprises a position corresponding to: position 126 according to SEQ ID NO:29, or the complement thereof; or position 126 according to SEQ ID NO:66, or the complement thereof. In some embodiments, the portion comprises positions corresponding to: positions 126-128 according to SEQ ID NO:29, or the complement thereof; and/or positions 126-128 according to SEQ ID NO:66, or the complement thereof.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of a Solute Carrier Family 9 Isoform A3 Regulatory Factor 2 missense nucleic acid molecule encoding a SLC9A3R2 predicted loss-of-function polypeptide, or the complement thereof. In some embodiments, the portion comprises a position corresponding to: position 9,519 according to SEQ ID NO:2, or the complement thereof.

In some embodiments, the portion comprises positions corresponding to: positions 9,519-9,521 according to SEQ ID NO:2, or the complement thereof.

In some embodiments, the portion comprises a position corresponding to: position 615 according to SEQ ID NO:22, or the complement thereof; position 589 according to SEQ ID NO:23, or the complement thereof; position 353 according to SEQ ID NO:24, or the complement thereof; position 230 according to SEQ ID NO:25, or the complement thereof; position 236 according to SEQ ID NO:26, or the complement thereof; position 236 according to SEQ ID NO:27, or the complement thereof; position 604 according to SEQ ID NO:28, or the complement thereof; or position 126 according to SEQ ID NO:29, or the complement thereof.

In some embodiments, the portion comprises positions corresponding to: positions 615-617 according to SEQ ID NO:22, or the complement thereof; positions 589-591 according to SEQ ID NO:23, or the complement thereof; positions 353-355 according to SEQ ID NO:24, or the complement thereof; positions 230-232 according to SEQ ID NO:25, or the complement thereof; positions 236-238 according to SEQ ID NO:26, or the complement thereof; positions 236-238 according to SEQ ID NO:27, or the complement thereof; positions 604-606 according to SEQ ID NO:28, or the complement thereof; or positions 126-128 according to SEQ ID NO:29, or the complement thereof.

In some embodiments, the portion comprises a position corresponding to: position 615 according to SEQ ID NO:59, or the complement thereof; position 589 according to SEQ ID NO:60, or the complement thereof; position 353 according to SEQ ID NO:61, or the complement thereof; position 230 according to SEQ ID NO:62, or the complement thereof; position 236 according to SEQ ID NO:63, or the complement thereof; position 236 according to SEQ ID NO:64, or the complement thereof; position 604 according to SEQ ID NO:65, or the complement thereof; or position 126 according to SEQ ID NO:66, or the complement thereof.

In some embodiments, the portion comprises positions corresponding to: positions 615-617 according to SEQ ID NO:59, or the complement thereof; positions 589-591 according to SEQ ID NO:60, or the complement thereof; positions 353-355 according to SEQ ID NO:61, or the complement thereof; positions 230-232 according to SEQ ID NO:62, or the complement thereof; positions 236-238 according to SEQ ID NO:63, or the complement thereof; positions 236-238 according to SEQ ID NO:64, or the complement thereof; positions 604-606 according to SEQ ID NO:65, or the complement thereof; or positions 126-128 according to SEQ ID NO:66, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the SLC9A3R2 missense variant genomic nucleic acid molecules, SLC9A3R2 missense variant mRNA molecules, and/or SLC9A3R2 missense variant cDNA molecules disclosed herein. The primers described herein can be used to amplify the SLC9A3R2 missense variant genomic nucleic acid molecules, SLC9A3R2 missense variant mRNA molecules, or SLC9A3R2 missense variant cDNA molecules, or a fragment thereof.

The present disclosure also provides pairs of primers comprising any of the primers described above. For example, if one of the primers' 3′-ends hybridizes to cytosine at a position corresponding to position 9,519 according to SEQ ID NO:1 (rather than a thymine) in a particular SLC9A3R2 nucleic acid molecule, then the presence of the amplified fragment would indicate the presence of an SLC9A3R2 reference genomic nucleic acid molecule. Conversely, if one of the primers' 3′-ends hybridizes to a thymine at a position corresponding to position 9,519 according to SEQ ID NO:2 (rather than cytosine) in a particular SLC9A3R2 nucleic acid molecule, then the presence of the amplified fragment would indicate the presence of the SLC9A3R2 missense variant genomic nucleic acid molecule. In some embodiments, the nucleotide of the primer complementary to the thymine at a position corresponding to position 9,519 according to SEQ ID NO:2 can be at the 3′ end of the primer. In addition, if one of the primers' 3′-ends hybridizes to a cytosine at a position corresponding to position 615 according to SEQ ID NO:3 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 615 according to SEQ ID NO:22 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 615 according to SEQ ID NO:22 can be at the 3′ end of the primer. In addition, if one of the primers' 3′-ends hybridizes to a cytosine at a position corresponding to position 615 according to SEQ ID NO:40 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 615 according to SEQ ID NO:59 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 615 according to SEQ ID NO:59 can be at the 3′ end of the primer.

The present disclosure also provides pairs of primers comprising any of the primers described above. For example, if one of the primers' 3′-ends hybridizes to a cytosine at a position corresponding to position 589 according to SEQ ID NO:4 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 589 according to SEQ ID NO:23 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 589 according to SEQ ID NO:23 can be at the 3′ end of the primer. In addition, if one of the primers' 3′-ends hybridizes to a cytosine at a position corresponding to position 589 according to SEQ ID NO:41 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 589 according to SEQ ID NO:60 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 589 according to SEQ ID NO:60 can be at the 3′ end of the primer.

The present disclosure also provides pairs of primers comprising any of the primers described above. For example, if one of the primers' 3′-ends hybridizes to a cytosine at a position corresponding to position 353 according to SEQ ID NO:5 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 353 according to SEQ ID NO:24 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 353 according to SEQ ID NO:24 can be at the 3′ end of the primer. In addition, if one of the primers' 3′-ends hybridizes to a cytosine at a position corresponding to position 353 according to SEQ ID NO:42 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 353 according to SEQ ID NO:61 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 353 according to SEQ ID NO:61 can be at the 3′ end of the primer.

The present disclosure also provides pairs of primers comprising any of the primers described above. For example, if one of the primers' 3′-ends hybridizes to a cytosine at a position corresponding to position 230 according to SEQ ID NO:6 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 230 according to SEQ ID NO:25 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 230 according to SEQ ID NO:25 can be at the 3′ end of the primer. In addition, if one of the primers' 3′-ends hybridizes to a cytosine at a position corresponding to position 230 according to SEQ ID NO:43 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 230 according to SEQ ID NO:62 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 230 according to SEQ ID NO:62 can be at the 3′ end of the primer.

The present disclosure also provides pairs of primers comprising any of the primers described above. For example, if one of the primers' 3′-ends hybridizes to a cytosine at a position corresponding to position 236 according to SEQ ID NO:7 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 236 according to SEQ ID NO:26 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 236 according to SEQ ID NO:26 can be at the 3′ end of the primer. In addition, if one of the primers' 3′-ends hybridizes to a cytosine at a position corresponding to position 236 according to SEQ ID NO:44 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 236 according to SEQ ID NO:63 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 236 according to SEQ ID NO:63 can be at the 3′ end of the primer.

The present disclosure also provides pairs of primers comprising any of the primers described above. For example, if one of the primers' 3′-ends hybridizes to a cytosine at a position corresponding to position 236 according to SEQ ID NO:8 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 236 according to SEQ ID NO:27 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 236 according to SEQ ID NO:27 can be at the 3′ end of the primer. In addition, if one of the primers' 3′-ends hybridizes to a cytosine at a position corresponding to position 236 according to SEQ ID NO:45 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 236 according to SEQ ID NO:64 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 236 according to SEQ ID NO:64 can be at the 3′ end of the primer.

The present disclosure also provides pairs of primers comprising any of the primers described above. For example, if one of the primers' 3′-ends hybridizes to a cytosine at a position corresponding to position 604 according to SEQ ID NO:9 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 604 according to SEQ ID NO:28 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 604 according to SEQ ID NO:28 can be at the 3′ end of the primer. In addition, if one of the primers' 3′-ends hybridizes to a cytosine at a position corresponding to position 604 according to SEQ ID NO:46 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 604 according to SEQ ID NO:65 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 604 according to SEQ ID NO:65 can be at the 3′ end of the primer.

The present disclosure also provides pairs of primers comprising any of the primers described above. For example, if one of the primers' 3′-ends hybridizes to a cytosine at a position corresponding to position 126 according to SEQ ID NO:10 (rather than a uracil) in a particular SLC9A3R2 nucleic acid molecule, then the presence of the amplified fragment would indicate the presence of an SLC9A3R2 reference mRNA molecule. Conversely, if one of the primers' 3′-ends hybridizes to a uracil at a position corresponding to position 126 according to SEQ ID NO:29 (rather than a cytosine) in a particular SLC9A3R2 mRNA molecule, then the presence of the amplified fragment would indicate the presence of the SLC9A3R2 missense variant mRNA molecule. In some embodiments, the nucleotide of the primer complementary to the uracil at a position corresponding to position 126 according to SEQ ID NO:29 can be at the 3′ end of the primer. In addition, if one of the primers' 3′-ends hybridizes to a cytosine at a position corresponding to position 126 according to SEQ ID NO:47 (rather than a thymine) in a particular SLC9A3R2 nucleic acid molecule, then the presence of the amplified fragment would indicate the presence of an SLC9A3R2 reference cDNA molecule. Conversely, if one of the primers' 3′-ends hybridizes to a thymine at a position corresponding to position 126 according to SEQ ID NO:66 (rather than a cytosine) in a particular SLC9A3R2 cDNA molecule, then the presence of the amplified fragment would indicate the presence of the SLC9A3R2 missense variant cDNA molecule. In some embodiments, the nucleotide of the primer complementary to the thymine at a position corresponding to position 126 according to SEQ ID NO:66 can be at the 3′ end of the primer.

In the context of the present disclosure “specifically hybridizes” means that the probe or primer (such as, for example, the alteration-specific probe or alteration-specific primer) does not hybridize to a nucleotide sequence encoding an SLC9A3R2 reference genomic nucleic acid molecule, an SLC9A3R2 reference mRNA molecule, and/or an SLC9A3R2 reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The present disclosure also provides molecular complexes comprising or consisting of any of the SLC9A3R2 missense nucleic acid molecules (genomic nucleic acid molecules, mRNA molecules, or cDNA molecules), or complement thereof, described herein and any of the alteration-specific primers or alteration-specific probes described herein. In some embodiments, the SLC9A3R2 missense nucleic acid molecules (genomic nucleic acid molecules, mRNA molecules, or cDNA molecules), or complement thereof, in the molecular complexes are single-stranded. In some embodiments, the SLC9A3R2 missense nucleic acid molecule is any of the genomic nucleic acid molecules described herein. In some embodiments, the SLC9A3R2 missense nucleic acid molecule is any of the mRNA molecules described herein. In some embodiments, the SLC9A3R2 missense nucleic acid molecule is any of the cDNA molecules described herein. In some embodiments, the molecular complex comprises or consists of any of the SLC9A3R2 missense nucleic acid molecules (genomic nucleic acid molecules, mRNA molecules, or cDNA molecules), or complement thereof, described herein and any of the alteration-specific primers described herein. In some embodiments, the molecular complex comprises or consists of any of the SLC9A3R2 missense nucleic acid molecules (genomic nucleic acid molecules, mRNA molecules, or cDNA molecules), or complement thereof, described herein and any of the alteration-specific probes described herein.

In some embodiments, the molecular complex comprises an alteration-specific primer or an alteration-specific probe hybridized to an SLC9A3R2 genomic nucleic acid molecule encoding an SLC9A3R2 predicted loss-of-function polypeptide, wherein the alteration-specific primer or the alteration-specific probe is hybridized to the SLC9A3R2 genomic nucleic acid molecule at a position corresponding to: position 9,519 according to SEQ ID NO:2, or the complement thereof.

In some embodiments, the alteration-specific primer or the alteration-specific probe in the molecular complex is hybridized to: TGG codon at positions corresponding to positions 9,519-9,521 according to SEQ ID NO:2.

In some embodiments, the genomic nucleic acid molecule in the molecular complex comprises SEQ ID NO:2.

In some embodiments, the molecular complex comprises an alteration-specific primer or an alteration-specific probe hybridized to an SLC9A3R2 mRNA molecule encoding an SLC9A3R2 predicted loss-of-function polypeptide, wherein the alteration-specific primer or the alteration-specific probe is hybridized to the SLC9A3R2 mRNA molecule at a position corresponding to: position 615 according to SEQ ID NO:22, or the complement thereof; position 589 according to SEQ ID NO:23, or the complement thereof; position 353 according to SEQ ID NO:24, or the complement thereof; position 230 according to SEQ ID NO:25, or the complement thereof; position 236 according to SEQ ID NO:26, or the complement thereof; position 236 according to SEQ ID NO:27, or the complement thereof; position 604 according to SEQ ID NO:28, or the complement thereof; or position 126 according to SEQ ID NO:29, or the complement thereof.

In some embodiments, the alteration-specific primer or the alteration-specific probe in the molecular complex is hybridized to: a UGG codon at positions corresponding to positions 615-617 according to SEQ ID NO:22, a UGG codon at positions corresponding to positions 589-591 according to SEQ ID NO:23, a UGG codon at positions corresponding to positions 353-355 according to SEQ ID NO:24, a UGG codon at positions corresponding to positions 230-232 according to SEQ ID NO:25, a UGG codon at positions corresponding to positions 236-238 according to SEQ ID NO:26, a UGG codon at positions corresponding to positions 236-238 according to SEQ ID NO:27, a UGG codon at positions corresponding to positions 604-606 according to SEQ ID NO:28, or a UGG codon at positions corresponding to positions 126-128 according to SEQ ID NO:29.

In some embodiments, the mRNA molecule in the molecular complex comprises SEQ ID NO:22, SEQ ID NO:23, SEQ ID NO:24, SEQ ID NO:25, SEQ ID NO:26, SEQ ID NO:27, SEQ ID NO:28, or SEQ ID NO:29.

In some embodiments, the molecular complex comprises an alteration-specific primer or an alteration-specific probe hybridized to an SLC9A3R2 cDNA molecule encoding an SLC9A3R2 predicted loss-of-function polypeptide, wherein the alteration-specific primer or the alteration-specific probe is hybridized to the SLC9A3R2 cDNA molecule at a position corresponding to: position 615 according to SEQ ID NO:59, or the complement thereof; position 589 according to SEQ ID NO:60, or the complement thereof; position 353 according to SEQ ID NO:61, or the complement thereof; position 230 according to SEQ ID NO:62, or the complement thereof; position 236 according to SEQ ID NO:63, or the complement thereof; position 236 according to SEQ ID NO:64, or the complement thereof; position 604 according to SEQ ID NO:65, or the complement thereof; or position 126 according to SEQ ID NO:66, or the complement thereof.

In some embodiments, the alteration-specific primer or the alteration-specific probe in the molecular complex is hybridized to: a TGG codon at positions corresponding to positions 615-617 according to SEQ ID NO:59, a TGG codon at positions corresponding to positions 589-591 according to SEQ ID NO:60, a TGG codon at positions corresponding to positions 353-355 according to SEQ ID NO:61, a TGG codon at positions corresponding to positions 230-232 according to SEQ ID NO:62, a TGG codon at positions corresponding to positions 236-238 according to SEQ ID NO:63, a TGG codon at positions corresponding to positions 236-238 according to SEQ ID NO:64, a TGG codon at positions corresponding to positions 604-606 according to SEQ ID NO:65, or a TGG codon at positions corresponding to positions 126-128 according to SEQ ID NO:66.

In some embodiments, the cDNA molecule in the molecular complex comprises SEQ ID NO:59, SEQ ID NO:60, SEQ ID NO:61, SEQ ID NO:62, SEQ ID NO:63, SEQ ID NO:64, SEQ ID NO:65, or SEQ ID NO:66.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of an SLC9A3R2 reference genomic nucleic acid molecule is set forth in SEQ ID NO:1 (ENSG00000065054.14 encompassing chr16:2,026,902-2,039,026 in the GRCh38/hg38 human genome assembly). Referring to SEQ ID NO:1, position 9,519 is cytosine.

A SLC9A3R2 missense variant genomic nucleic acid molecule exists, wherein the cytosine at position 9,519 is replaced with a thymine. The nucleotide sequence of this SLC9A3R2 missense variant genomic nucleic acid molecule is set forth in SEQ ID NO:2.

The nucleotide sequence of an SLC9A3R2 reference mRNA molecule is set forth in SEQ ID NO:3. Referring to SEQ ID NO:3, position 615 is a cytosine. The nucleotide sequence of another SLC9A3R2 reference mRNA molecule is set forth in SEQ ID NO:4. Referring to SEQ ID NO:4, position 589 is a cytosine. The nucleotide sequence of another SLC9A3R2 reference mRNA molecule is set forth in SEQ ID NO:5. Referring to SEQ ID NO:5, position 353 is a cytosine. The nucleotide sequence of another SLC9A3R2 reference mRNA molecule is set forth in SEQ ID NO:6. Referring to SEQ ID NO:6, position 230 is a cytosine. The nucleotide sequence of another SLC9A3R2 reference mRNA molecule is set forth in SEQ ID NO:7. Referring to SEQ ID NO:7, position 236 is a cytosine. The nucleotide sequence of another SLC9A3R2 reference mRNA molecule is set forth in SEQ ID NO:7. Referring to SEQ ID NO:7, position 236 is a cytosine. The nucleotide sequence of another SLC9A3R2 reference mRNA molecule is set forth in SEQ ID NO:8. Referring to SEQ ID NO:8, position 236 is a cytosine. The nucleotide sequence of another SLC9A3R2 reference mRNA molecule is set forth in SEQ ID NO:9. Referring to SEQ ID NO:9, position 604 is a cytosine. The nucleotide sequence of another SLC9A3R2 reference mRNA molecule is set forth in SEQ ID NO:10. Referring to SEQ ID NO:10, position 126 is a cytosine. The nucleotide sequence of another SLC9A3R2 reference mRNA molecule is set forth in SEQ ID NO:11. The nucleotide sequence of another SLC9A3R2 reference mRNA molecule is set forth in SEQ ID NO:12. Referring to SEQ ID NO:12, position 625 is a cytosine. The nucleotide sequence of another SLC9A3R2 reference mRNA molecule is set forth in SEQ ID NO:13. Referring to SEQ ID NO:13, position 622 is a cytosine. The nucleotide sequence of another SLC9A3R2 reference mRNA molecule is set forth in SEQ ID NO:14. Referring to SEQ ID NO:14, position 618 is a cytosine. The nucleotide sequence of another SLC9A3R2 reference mRNA molecule is set forth in SEQ ID NO:15. Referring to SEQ ID NO:15, position 527 is a cytosine. The nucleotide sequence of another SLC9A3R2 reference mRNA molecule is set forth in SEQ ID NO:16. Referring to SEQ ID NO:16, position 511 is a cytosine. The nucleotide sequence of another SLC9A3R2 reference mRNA molecule is set forth in SEQ ID NO:17. Referring to SEQ ID NO:17, position 649 is a cytosine. The nucleotide sequence of another SLC9A3R2 reference mRNA molecule is set forth in SEQ ID NO:18. Referring to SEQ ID NO:18, position 615 is a cytosine. The nucleotide sequence of another SLC9A3R2 reference mRNA molecule is set forth in SEQ ID NO:19 Referring to SEQ ID NO:19, position 602 is a cytosine. The nucleotide sequence of another SLC9A3R2 reference mRNA molecule is set forth in SEQ ID NO:20. Referring to SEQ ID NO:20, position 260 is a cytosine. The nucleotide sequence of another SLC9A3R2 reference mRNA molecule is set forth in SEQ ID NO:21. Referring to SEQ ID NO:21, position 259 is a cytosine.

A SLC9A3R2 missense variant mRNA molecule exists, wherein the cytosine at position 615 is replaced with a uracil. The nucleotide sequence of this SLC9A3R2 missense variant mRNA molecule is set forth in SEQ ID NO:22.

Another SLC9A3R2 missense variant mRNA molecule exists, wherein the cytosine at position 589 is replaced with a uracil. The nucleotide sequence of this SLC9A3R2 missense variant mRNA molecule is set forth in SEQ ID NO:23.

Another SLC9A3R2 missense variant mRNA molecule exists, wherein the cytosine at position 353 is replaced with a uracil. The nucleotide sequence of this SLC9A3R2 missense variant mRNA molecule is set forth in SEQ ID NO:24.

Another SLC9A3R2 missense variant mRNA molecule exists, wherein the cytosine at position 230 is replaced with a uracil. The nucleotide sequence of this SLC9A3R2 missense variant mRNA molecule is set forth in SEQ ID NO:25.

Another SLC9A3R2 missense variant mRNA molecule exists, wherein the cytosine at position 236 is replaced with a uracil. The nucleotide sequence of this SLC9A3R2 missense variant mRNA molecule is set forth in SEQ ID NO:26.

Another SLC9A3R2 missense variant mRNA molecule exists, wherein the cytosine at position 236 is replaced with a uracil. The nucleotide sequence of this SLC9A3R2 missense variant mRNA molecule is set forth in SEQ ID NO:27.

Another SLC9A3R2 missense variant mRNA molecule exists, wherein the cytosine at position 604 is replaced with a uracil. The nucleotide sequence of this SLC9A3R2 missense variant mRNA molecule is set forth in SEQ ID NO:28.

Another SLC9A3R2 missense variant mRNA molecule exists, wherein the cytosine at position 126 is replaced with a uracil. The nucleotide sequence of this SLC9A3R2 missense variant mRNA molecule is set forth in SEQ ID NO:29.

Another SLC9A3R2 missense variant mRNA molecule exists, wherein the cytosine at position 625 is replaced with a uracil. The nucleotide sequence of this SLC9A3R2 missense variant mRNA molecule is set forth in SEQ ID NO:30.

Another SLC9A3R2 missense variant mRNA molecule exists, wherein the cytosine at position 622 is replaced with a uracil. The nucleotide sequence of this SLC9A3R2 missense variant mRNA molecule is set forth in SEQ ID NO:31.

Another SLC9A3R2 missense variant mRNA molecule exists, wherein the cytosine at position 618 is replaced with a uracil. The nucleotide sequence of this SLC9A3R2 missense variant mRNA molecule is set forth in SEQ ID NO:32.

Another SLC9A3R2 missense variant mRNA molecule exists, wherein the cytosine at position 527 is replaced with a uracil. The nucleotide sequence of this SLC9A3R2 missense variant mRNA molecule is set forth in SEQ ID NO:33.

Another SLC9A3R2 missense variant mRNA molecule exists, wherein the cytosine at position 511 is replaced with a uracil. The nucleotide sequence of this SLC9A3R2 missense variant mRNA molecule is set forth in SEQ ID NO:34.

Another SLC9A3R2 missense variant mRNA molecule exists, wherein the cytosine at position 649 is replaced with a uracil. The nucleotide sequence of this SLC9A3R2 missense variant mRNA molecule is set forth in SEQ ID NO:35.

Another SLC9A3R2 missense variant mRNA molecule exists, wherein the cytosine at position 615 is replaced with a uracil. The nucleotide sequence of this SLC9A3R2 missense variant mRNA molecule is set forth in SEQ ID NO:36.

Another SLC9A3R2 missense variant mRNA molecule exists, wherein the cytosine at position 602 is replaced with a uracil. The nucleotide sequence of this SLC9A3R2 missense variant mRNA molecule is set forth in SEQ ID NO:37.

Another SLC9A3R2 missense variant mRNA molecule exists, wherein the cytosine at position 260 is replaced with a uracil. The nucleotide sequence of this SLC9A3R2 missense variant mRNA molecule is set forth in SEQ ID NO:38.

Another SLC9A3R2 missense variant mRNA molecule exists, wherein the cytosine at position 259 is replaced with a uracil. The nucleotide sequence of this SLC9A3R2 missense variant mRNA molecule is set forth in SEQ ID NO:39.

The nucleotide sequence of an SLC9A3R2 reference cDNA molecule is set forth in SEQ ID NO:40. Referring to SEQ ID NO:40, position 615 is a cytosine. The nucleotide sequence of another SLC9A3R2 reference cDNA molecule is set forth in SEQ ID NO:41. Referring to SEQ ID NO:41 position 589 is a cytosine. The nucleotide sequence of another SLC9A3R2 reference cDNA molecule is set forth in SEQ ID NO:42. Referring to SEQ ID NO:42, position 353 is a cytosine. The nucleotide sequence of another SLC9A3R2 reference cDNA molecule is set forth in SEQ ID NO:43. Referring to SEQ ID NO:43, position 230 is a cytosine. The nucleotide sequence of another SLC9A3R2 reference cDNA molecule is set forth in SEQ ID NO:44. Referring to SEQ ID NO:44, position 236 is a cytosine. The nucleotide sequence of another SLC9A3R2 reference cDNA molecule is set forth in SEQ ID NO:45. Referring to SEQ ID NO:45, position 236 is a cytosine. The nucleotide sequence of another SLC9A3R2 reference cDNA molecule is set forth in SEQ ID NO:46. Referring to SEQ ID NO:46, position 604 is a cytosine. The nucleotide sequence of another SLC9A3R2 reference cDNA molecule is set forth in SEQ ID NO:47. Referring to SEQ ID NO:47, position 126 is a cytosine. The nucleotide sequence of another SLC9A3R2 reference cDNA molecule is set forth in SEQ ID NO:48. The nucleotide sequence of another SLC9A3R2 reference cDNA molecule is set forth in SEQ ID NO:49. Referring to SEQ ID NO:49, position 625 is a cytosine. The nucleotide sequence of another SLC9A3R2 reference cDNA molecule is set forth in SEQ ID NO:50. Referring to SEQ ID NO:50, position 622 is a cytosine. The nucleotide sequence of another SLC9A3R2 reference cDNA molecule is set forth in SEQ ID NO:51. Referring to SEQ ID NO:51, position 618 is a cytosine. The nucleotide sequence of another SLC9A3R2 reference cDNA molecule is set forth in SEQ ID NO:52. Referring to SEQ ID NO:52, position 527 is a cytosine. The nucleotide sequence of another SLC9A3R2 reference cDNA molecule is set forth in SEQ ID NO:53. Referring to SEQ ID NO:53, position 511 is a cytosine. The nucleotide sequence of another SLC9A3R2 reference cDNA molecule is set forth in SEQ ID NO:54. Referring to SEQ ID NO:54, position 649 is a cytosine. The nucleotide sequence of another SLC9A3R2 reference cDNA molecule is set forth in SEQ ID NO:55. Referring to SEQ ID NO:55, position 615 is a cytosine. The nucleotide sequence of another SLC9A3R2 reference cDNA molecule is set forth in SEQ ID NO:56. Referring to SEQ ID NO:56, position 602 is a cytosine. The nucleotide sequence of another SLC9A3R2 reference cDNA molecule is set forth in SEQ ID NO:57. Referring to SEQ ID NO:57, position 260 is a cytosine. The nucleotide sequence of another SLC9A3R2 reference cDNA molecule is set forth in SEQ ID NO:58. Referring to SEQ ID NO:58, position 259 is a cytosine.

A SLC9A3R2 missense variant cDNA molecule exists, wherein the cytosine at position 615 is replaced with a thymine. The nucleotide sequence of this SLC9A3R2 missense variant cDNA molecule is set forth in SEQ ID NO:59.

Another SLC9A3R2 missense variant cDNA molecule exists, wherein the cytosine at position 589 is replaced with a thymine. The nucleotide sequence of this SLC9A3R2 missense variant cDNA molecule is set forth in SEQ ID NO:60.

Another SLC9A3R2 missense variant cDNA molecule exists, wherein the cytosine at position 353 is replaced with a thymine. The nucleotide sequence of this SLC9A3R2 missense variant cDNA molecule is set forth in SEQ ID NO:61.

Another SLC9A3R2 missense variant cDNA molecule exists, wherein the cytosine at position 230 is replaced with a thymine. The nucleotide sequence of this SLC9A3R2 missense variant cDNA molecule is set forth in SEQ ID NO:62.

Another SLC9A3R2 missense variant cDNA molecule exists, wherein the cytosine at position 236 is replaced with a thymine. The nucleotide sequence of this SLC9A3R2 missense variant cDNA molecule is set forth in SEQ ID NO:63.

Another SLC9A3R2 missense variant cDNA molecule exists, wherein the cytosine at position 236 is replaced with a thymine. The nucleotide sequence of this SLC9A3R2 missense variant cDNA molecule is set forth in SEQ ID NO:64.

Another SLC9A3R2 missense variant cDNA molecule exists, wherein the cytosine at position 604 is replaced with a thymine. The nucleotide sequence of this SLC9A3R2 missense variant cDNA molecule is set forth in SEQ ID NO:65.

Another SLC9A3R2 missense variant cDNA molecule exists, wherein the cytosine at position 126 is replaced with a thymine. The nucleotide sequence of this SLC9A3R2 missense variant cDNA molecule is set forth in SEQ ID NO:66.

Another SLC9A3R2 missense variant cDNA molecule exists, wherein the cytosine at position 625 is replaced with a thymine. The nucleotide sequence of this SLC9A3R2 missense variant cDNA molecule is set forth in SEQ ID NO:67.

Another SLC9A3R2 missense variant cDNA molecule exists, wherein the cytosine at position 622 is replaced with a thymine. The nucleotide sequence of this SLC9A3R2 missense variant cDNA molecule is set forth in SEQ ID NO:68.

Another SLC9A3R2 missense variant cDNA molecule exists, wherein the cytosine at position 618 is replaced with a thymine. The nucleotide sequence of this SLC9A3R2 missense variant cDNA molecule is set forth in SEQ ID NO:69.

Another SLC9A3R2 missense variant cDNA molecule exists, wherein the cytosine at position 527 is replaced with a thymine. The nucleotide sequence of this SLC9A3R2 missense variant cDNA molecule is set forth in SEQ ID NO:70.

Another SLC9A3R2 missense variant cDNA molecule exists, wherein the cytosine at position 511 is replaced with a thymine. The nucleotide sequence of this SLC9A3R2 missense variant cDNA molecule is set forth in SEQ ID NO:71.

Another SLC9A3R2 missense variant cDNA molecule exists, wherein the cytosine at position 649 is replaced with a thymine. The nucleotide sequence of this SLC9A3R2 missense variant cDNA molecule is set forth in SEQ ID NO:72.

Another SLC9A3R2 missense variant cDNA molecule exists, wherein the cytosine at position 615 is replaced with a thymine. The nucleotide sequence of this SLC9A3R2 missense variant cDNA molecule is set forth in SEQ ID NO:73.

Another SLC9A3R2 missense variant cDNA molecule exists, wherein the cytosine at position 602 is replaced with a thymine. The nucleotide sequence of this SLC9A3R2 missense variant cDNA molecule is set forth in SEQ ID NO:74.

Another SLC9A3R2 missense variant cDNA molecule exists, wherein the cytosine at position 602 is replaced with a thymine. The nucleotide sequence of this SLC9A3R2 missense variant cDNA molecule is set forth in SEQ ID NO:75.

Another SLC9A3R2 missense variant cDNA molecule exists, wherein the cytosine at position 259 is replaced with a thymine. The nucleotide sequence of this SLC9A3R2 missense variant cDNA molecule is set forth in SEQ ID NO:76.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein, or vectors comprising the same. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:40). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, an SLC9A3R2 missense nucleic acid molecule comprising a nucleotide sequence encoding an SLC9A3R2 predicted loss-of-function polypeptide, wherein the nucleotide sequence comprises a thymine at a position corresponding to position 9,519 according to SEQ ID NO:2 means that if the nucleotide sequence of the SLC9A3R2 genomic nucleic acid molecule is aligned to the sequence of SEQ ID NO:2, the SLC9A3R2 sequence has a thymine residue at the position that corresponds to position 9,519 of SEQ ID NO:2. The same applies for SLC9A3R2 mRNA molecules comprising a nucleotide sequence encoding an SLC9A3R2 predicted loss-of-function polypeptide, wherein the nucleotide sequence comprises a uracil at a position corresponding to position 615 according to SEQ ID NO:22, and SLC9A3R2 cDNA molecules comprising a nucleotide sequence encoding an SLC9A3R2 predicted loss-of-function polypeptide, wherein the nucleotide sequence comprises a thymine at a position corresponding to position 615 according to SEQ ID NO:59. These phrases refer to a an SLC9A3R2 missense nucleic acid molecule encoding an SLC9A3R2 predicted loss-of-function polypeptide, wherein the genomic nucleic acid molecule has a nucleotide sequence that comprises a thymine residue that is homologous to the thymine residue at position 9,519 of SEQ ID NO:2 (or wherein the mRNA molecule has a nucleotide sequence that comprises a uracil residue that is homologous to the uracil residue at position 615 of SEQ ID NO:22, or wherein the cDNA molecule has a nucleotide sequence that comprises a thymine residue that is homologous to the thymine residue at position 615 of SEQ ID NO:59). Herein, such a sequence is also referred to as “SLC9A3R2 sequence with the Arg171Trp-Long alteration” or “SLC9A3R2 sequence with the Arg171Trp-Long variation.”

As described herein, a position within an SLC9A3R2 missense genomic nucleic acid molecule that corresponds to position 9,519 according to SEQ ID NO:2, for example, can be identified by performing a sequence alignment between the nucleotide sequence of a particular SLC9A3R2 nucleic acid molecule and the nucleotide sequence of SEQ ID NO:2. A variety of computational algorithms exist that can be used for performing a sequence alignment to identify a nucleotide position that corresponds to, for example, position 9,519 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of an SLC9A3R2 reference polypeptide is set forth in SEQ ID NO:77. Referring to SEQ ID NO:77, the SLC9A3R2 reference polypeptide is 337 amino acids in length. Referring SEQ ID NO:77, position 171 is an arginine.

The amino acid sequence of anther SLC9A3R2 reference polypeptide is set forth in SEQ ID NO:78. Referring to SEQ ID NO:78, the SLC9A3R2 reference polypeptide is 326 amino acids in length. Referring to SEQ ID NO:78, position 171 is an arginine.

The amino acid sequence of anther SLC9A3R2 reference polypeptide is set forth in SEQ ID NO:79. Referring to SEQ ID NO:79, the SLC9A3R2 reference polypeptide is 231 amino acids in length. Referring to SEQ ID NO:79, position 65 is an arginine.

The amino acid sequence of anther SLC9A3R2 reference polypeptide is set forth in SEQ ID NO:80. Referring to SEQ ID NO:80, the SLC9A3R2 reference polypeptide is 224 amino acids in length. Referring to SEQ ID NO:80, position 58 is an arginine.

The amino acid sequence of anther SLC9A3R2 reference polypeptide is set forth in SEQ ID NO:81. Referring to SEQ ID NO:81, the SLC9A3R2 reference polypeptide is 215 amino acids in length. Referring to SEQ ID NO:81, position 60 is an arginine.

The amino acid sequence of anther SLC9A3R2 reference polypeptide is set forth in SEQ ID NO:82. Referring to SEQ ID NO:82, the SLC9A3R2 reference polypeptide is 226 amino acids in length. Referring to SEQ ID NO:82, position 60 is an arginine.

The amino acid sequence of anther SLC9A3R2 reference polypeptide is set forth in SEQ ID NO:83. Referring to SEQ ID NO:83, the SLC9A3R2 reference polypeptide is 450 amino acids in length. Referring to SEQ ID NO:83, position 170 is an arginine.

The amino acid sequence of anther SLC9A3R2 reference polypeptide is set forth in SEQ ID NO:84. Referring to SEQ ID NO:84, the SLC9A3R2 reference polypeptide is 122 amino acids in length.

The amino acid sequence of anther SLC9A3R2 reference polypeptide is set forth in SEQ ID NO:85. Referring to SEQ ID NO:85, the SLC9A3R2 reference polypeptide is 151 amino acids in length.

A SLC9A3R2 predicted loss-of-function polypeptide exists (Arg171Trp-Long), the amino acid sequence of which is set forth in SEQ ID NO:86. Referring to SEQ ID NO:86, the SLC9A3R2 predicted loss-of-function polypeptide is 337 amino acids in length. Referring to SEQ ID NO:86, position 171 is tryptophan.

Another SLC9A3R2 predicted loss-of-function polypeptide exists (Arg171Trp-Short), the amino acid sequence of which is set forth in SEQ ID NO:87. Referring to SEQ ID NO:87, the SLC9A3R2 predicted loss-of-function polypeptide is 326 amino acids in length. Referring to SEQ ID NO:87, position 171 is tryptophan.

Another SLC9A3R2 predicted loss-of-function polypeptide exists (Arg65Trp), the amino acid sequence of which is set forth in SEQ ID NO:88. Referring to SEQ ID NO:88, the SLC9A3R2 predicted loss-of-function polypeptide is 231 amino acids in length. Referring to SEQ ID NO:88, position 65 is tryptophan.

Another SLC9A3R2 predicted loss-of-function polypeptide exists (Arg58Trp), the amino acid sequence of which is set forth in SEQ ID NO:89. Referring to SEQ ID NO:89, the SLC9A3R2 predicted loss-of-function polypeptide is 224 amino acids in length. Referring to SEQ ID NO:89, position 58 is tryptophan.

Another SLC9A3R2 predicted loss-of-function polypeptide exists (Arg60Trp-Short), the amino acid sequence of which is set forth in SEQ ID NO:90. Referring to SEQ ID NO:90, the SLC9A3R2 predicted loss-of-function polypeptide is 215 amino acids in length. Referring to SEQ ID NO:90, position 60 is tryptophan.

Another SLC9A3R2 predicted loss-of-function polypeptide exists (Arg60Trp-Long), the amino acid sequence of which is set forth in SEQ ID NO:91. Referring to SEQ ID NO:91, the SLC9A3R2 predicted loss-of-function polypeptide is 226 amino acids in length. Referring to SEQ ID NO:91, position 60 is tryptophan.

Another SLC9A3R2 predicted loss-of-function polypeptide exists (Arg170Trp), the amino acid sequence of which is set forth in SEQ ID NO:92. Referring to SEQ ID NO:92, the SLC9A3R2 predicted loss-of-function polypeptide is 450 amino acids in length. Referring to SEQ ID NO:92, position 60 is tryptophan.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or prevent hypertension, coronary heart disease, and/or atrial fibrillation for use in the treatment or prevention of hypertension, coronary heart disease, and/or atrial fibrillation (or for use in the preparation of a medicament for treating or preventing hypertension, coronary heart disease, and/or atrial fibrillation) in a subject, wherein the subject has any of the SLC9A3R2 missense variant genomic nucleic acid molecules, missense variant mRNA molecules, and/or missense variant cDNA molecules encoding an SLC9A3R2 predicted loss-of-function polypeptide described herein. The therapeutic agents that treat or prevent hypertension, coronary heart disease, and/or atrial fibrillation can be any of the therapeutic agents that treat or prevent hypertension, coronary heart disease, and/or atrial fibrillation described herein. The hypertension can be any of primary hypertension, secondary hypertension, resistant hypertension, and malignant hypertension.

In some embodiments, the subject is identified as having a genomic nucleic acid molecule encoding an SLC9A3R2 predicted loss-of-function polypeptide, or the complement thereof, wherein the genomic nucleic acid molecule has a nucleotide sequence comprising a thymine at a position corresponding to position 9,519 according to SEQ ID NO:2, or the complement thereof.

In some embodiments, the subject is identified as having an mRNA molecule encoding an SLC9A3R2 predicted loss-of-function polypeptide, or the complement thereof, wherein the mRNA molecule has a nucleotide sequence comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof.

In some embodiments, the subject is identified as having a cDNA molecule encoding an SLC9A3R2 predicted loss-of-function polypeptide, or the complement thereof, wherein the cDNA molecule has a nucleotide sequence comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

In some embodiments, the subject is identified as having: a genomic nucleic acid molecule having a nucleotide sequence encoding an SLC9A3R2 predicted loss-of-function polypeptide, wherein the nucleotide sequence comprises a thymine at a position corresponding to position 9,519 according to SEQ ID NO:2, or the complement thereof; an mRNA molecule having a nucleotide sequence encoding an SLC9A3R2 predicted loss-of-function polypeptide, wherein the nucleotide sequence comprises a uracil at a position corresponding to position 615 according to SEQ ID NO:22, or the complement thereof; a cDNA molecule having a nucleotide sequence encoding an SLC9A3R2 predicted loss-of-function polypeptide, wherein the nucleotide sequence comprises a thymine at a position corresponding to position 615 according to SEQ ID NO:59, or the complement thereof; or an SLC9A3R2 predicted loss-of-function polypeptide that comprises tryptophan at a position corresponding to position 171 according to SEQ ID NO:86.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 589 according to SEQ ID NO:23, or the complement thereof; a cDNA molecule having a nucleotide sequence encoding an SLC9A3R2 predicted loss-of-function polypeptide, wherein the nucleotide sequence comprises a thymine at a position corresponding to position 589 according to SEQ ID NO:60, or the complement thereof; or an SLC9A3R2 predicted loss-of-function polypeptide that comprises tryptophan at a position corresponding to position 171 according to SEQ ID NO:87.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 353 according to SEQ ID NO:24, or the complement thereof; a cDNA molecule having a nucleotide sequence encoding an SLC9A3R2 predicted loss-of-function polypeptide, wherein the nucleotide sequence comprises a thymine at a position corresponding to position 353 according to SEQ ID NO:61, or the complement thereof; or an SLC9A3R2 predicted loss-of-function polypeptide that comprises tryptophan at a position corresponding to position 65 according to SEQ ID NO:88.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 230 according to SEQ ID NO:25, or the complement thereof; a cDNA molecule having a nucleotide sequence encoding an SLC9A3R2 predicted loss-of-function polypeptide, wherein the nucleotide sequence comprises a thymine at a position corresponding to position 230 according to SEQ ID NO:62, or the complement thereof; or an SLC9A3R2 predicted loss-of-function polypeptide that comprises tryptophan at a position corresponding to position 58 according to SEQ ID NO:89.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 236 according to SEQ ID NO:26, or the complement thereof; a cDNA molecule having a nucleotide sequence encoding an SLC9A3R2 predicted loss-of-function polypeptide, wherein the nucleotide sequence comprises a thymine at a position corresponding to position 236 according to SEQ ID NO:63, or the complement thereof; or an SLC9A3R2 predicted loss-of-function polypeptide that comprises tryptophan at a position corresponding to position 60 according to SEQ ID NO:90.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 236 according to SEQ ID NO:27, or the complement thereof; a cDNA molecule having a nucleotide sequence encoding an SLC9A3R2 predicted loss-of-function polypeptide, wherein the nucleotide sequence comprises a thymine at a position corresponding to position 236 according to SEQ ID NO:64, or the complement thereof; or an SLC9A3R2 predicted loss-of-function polypeptide that comprises tryptophan at a position corresponding to position 60 according to SEQ ID NO:91.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 604 according to SEQ ID NO:28, or the complement thereof; a cDNA molecule having a nucleotide sequence encoding an SLC9A3R2 predicted loss-of-function polypeptide, wherein the nucleotide sequence comprises a thymine at a position corresponding to position 604 according to SEQ ID NO:65, or the complement thereof; or an SLC9A3R2 predicted loss-of-function polypeptide that comprises tryptophan at a position corresponding to position 60 according to SEQ ID NO:92.

In some embodiments, the subject is identified as having: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 126 according to SEQ ID NO:29, or the complement thereof; or a cDNA molecule having a nucleotide sequence encoding an SLC9A3R2 predicted loss-of-function polypeptide, wherein the nucleotide sequence comprises a thymine at a position corresponding to position 126 according to SEQ ID NO:66, or the complement thereof.

The present disclosure also provides SLC9A3R2 inhibitors for use in the treatment or prevention of hypertension, coronary heart disease, and/or atrial fibrillation (or for use in the preparation of a medicament for treating or preventing hypertension, coronary heart disease, and/or atrial fibrillation) in a subject, wherein the subject is heterozygous for any of the SLC9A3R2 missense variant genomic nucleic acid molecules, missense variant mRNA molecules, and/or missense variant cDNA molecules encoding an SLC9A3R2 predicted loss-of-function polypeptides described herein, or wherein the subject is reference for an SLC9A3R2 genomic nucleic acid molecule, mRNA molecule, or cDNA molecule. The SLC9A3R2 inhibitors can be any of the SLC9A3R2 inhibitors described herein. The hypertension can be any of primary hypertension, secondary hypertension, resistant hypertension, and malignant hypertension.

In some embodiments, the subject is reference for an SLC9A3R2 genomic nucleic acid molecule, an SLC9A3R2 mRNA molecule, or an SLC9A3R2 cDNA molecule.

In some embodiments, the subject is heterozygous for a genomic nucleic acid molecule encoding an SLC9A3R2 predicted loss-of-function polypeptide, or the complement thereof, wherein the genomic nucleic acid molecule has a nucleotide sequence comprising a thymine at a position corresponding to position 9,519 according to SEQ ID NO:2, or the complement thereof.

In some embodiments, the subject is heterozygous for an mRNA molecule encoding an SLC9A3R2 predicted loss-of-function polypeptide, or the complement thereof, wherein the mRNA molecule has a nucleotide sequence comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof.

In some embodiments, the subject is heterozygous for a cDNA molecule encoding an SLC9A3R2 predicted loss-of-function polypeptide, or the complement thereof, wherein the cDNA molecule has a nucleotide sequence comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises a thymine at a position corresponding to position 9,519 according to SEQ ID NO:2, or the complement thereof; an mRNA molecule having a nucleotide sequence encoding an SLC9A3R2 predicted loss-of-function polypeptide, wherein the nucleotide sequence comprises a uracil at a position corresponding to position 615 according to SEQ ID NO:22, or the complement thereof; a cDNA molecule having a nucleotide sequence encoding an SLC9A3R2 predicted loss-of-function polypeptide, wherein the nucleotide sequence comprises a thymine at a position corresponding to position 615 according to SEQ ID NO:59, or the complement thereof; or an SLC9A3R2 predicted loss-of-function polypeptide that comprises tryptophan at a position corresponding to position 171 according to SEQ ID NO:86. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 589 according to SEQ ID NO:23, or the complement thereof; a cDNA molecule having a nucleotide sequence encoding an SLC9A3R2 predicted loss-of-function polypeptide, wherein the nucleotide sequence comprises a thymine at a position corresponding to position 589 according to SEQ ID NO:60, or the complement thereof; or an SLC9A3R2 predicted loss-of-function polypeptide that comprises tryptophan at a position corresponding to position 171 according to SEQ ID NO:87. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 353 according to SEQ ID NO:24, or the complement thereof; a cDNA molecule having a nucleotide sequence encoding an SLC9A3R2 predicted loss-of-function polypeptide, wherein the nucleotide sequence comprises a thymine at a position corresponding to position 353 according to SEQ ID NO:61, or the complement thereof; or an SLC9A3R2 predicted loss-of-function polypeptide that comprises tryptophan at a position corresponding to position 65 according to SEQ ID NO:88. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 230 according to SEQ ID NO:25, or the complement thereof; a cDNA molecule having a nucleotide sequence encoding an SLC9A3R2 predicted loss-of-function polypeptide, wherein the nucleotide sequence comprises a thymine at a position corresponding to position 230 according to SEQ ID NO:62, or the complement thereof; or an SLC9A3R2 predicted loss-of-function polypeptide that comprises tryptophan at a position corresponding to position 58 according to SEQ ID NO:89. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 236 according to SEQ ID NO:26, or the complement thereof; a cDNA molecule having a nucleotide sequence encoding an SLC9A3R2 predicted loss-of-function polypeptide, wherein the nucleotide sequence comprises a thymine at a position corresponding to position 236 according to SEQ ID NO:63, or the complement thereof; or an SLC9A3R2 predicted loss-of-function polypeptide that comprises tryptophan at a position corresponding to position 60 according to SEQ ID NO:90. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 236 according to SEQ ID NO:27, or the complement thereof; a cDNA molecule having a nucleotide sequence encoding an SLC9A3R2 predicted loss-of-function polypeptide, wherein the nucleotide sequence comprises a thymine at a position corresponding to position 236 according to SEQ ID NO:64, or the complement thereof; or an SLC9A3R2 predicted loss-of-function polypeptide that comprises tryptophan at a position corresponding to position 60 according to SEQ ID NO:91. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 604 according to SEQ ID NO:28, or the complement thereof; a cDNA molecule having a nucleotide sequence encoding an SLC9A3R2 predicted loss-of-function polypeptide, wherein the nucleotide sequence comprises a thymine at a position corresponding to position 604 according to SEQ ID NO:65, or the complement thereof; or an SLC9A3R2 predicted loss-of-function polypeptide that comprises tryptophan at a position corresponding to position 60 according to SEQ ID NO:92. The SLC9A3R2 inhibitors can be any of the SLC9A3R2 inhibitors described herein.

In some embodiments, the subject is identified as being heterozygous for: a genomic nucleic acid molecule having a nucleotide sequence encoding an SLC9A3R2 predicted loss-of-function polypeptide, wherein the nucleotide sequence comprises an mRNA molecule having a nucleotide sequence encoding an SLC9A3R2 predicted loss-of-function polypeptide, wherein the nucleotide sequence comprises a uracil at a position corresponding to position 126 according to SEQ ID NO:29, or the complement thereof; or a cDNA molecule having a nucleotide sequence encoding an SLC9A3R2 predicted loss-of-function polypeptide, wherein the nucleotide sequence comprises a thymine at a position corresponding to position 126 according to SEQ ID NO:66, or the complement thereof.

In some embodiments, the subject is identified as having: an SLC9A3R2 reference genomic nucleic acid molecule comprising SEQ ID NO:1, an SLC9A3R2 reference mRNA molecules comprising one or more SEQ ID NOs:3-21, an SLC9A3R2 reference cDNA molecules comprising one or more SEQ ID NOs:40-58, or an SLC9A3R2 reference polypeptide comprising one or more SEQ ID NOs:77-85. The SLC9A3R2 inhibitors can be any of the SLC9A3R2 inhibitors described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Rare pLOFs and Deleterious Missense Variants in SLC9A3R2 Associated with Lower Risk of Hypertension

The exomes of 454,787 UKB study participants were sequenced, with 95.8% of targeted bases covered at a depth of 20× or greater, as previously described (Szustakowski, Advancing Human Genetics Research and Drug Discovery through Exome Sequencing of the UK Biobank. bioRxiv, 2021; and Van Hout et al., Nature, 2020). Twelve million variants were identified in 39 million base pairs across the coding regions of 18,659 genes (data not shown). Among the variants identified were 3,375,252 (median of 10,260 per individual) synonymous 7,689,495 (9,284 per individual) missense and 889,957 (212 per individual) putative loss-of-function (pLOF) variants (data not shown), of which about half were observed only once in this dataset (singleton variants; data not shown).

A novel association was identified between a lower risk of hypertension and a burden of rare pLOFs and deleterious missense variants in SLC9A3R2 (5,873 carriers; OR=0.81, 95% CI 0.76 to 0.87, P=2.2×10⁻¹⁰). In addition, there was also an association with lower systolic blood pressure (SBP; effect=−1.85 mmHg, 95% CI=−2.22 to −1.48, P=2.0×10⁻¹⁹) and lower diastolic blood pressure (effect=−1.01 mmHg, 95% CI=−1.31 to −0.80, P=3.7×10⁻¹⁸), with the SBP association replicating in the GHS cohort (1,517 carriers; effect=−0.077 SD units, 95% CI −0.118 to −0.0356, P=2.6×10⁻⁴).

A low frequency missense variant in SLC9A3R2 (r5139491786, Arg171Trp, MAF=0.7%) was previously identified in a GWAS of blood pressure, but the signal was attributed to the nearby PKD1 gene variant (r5140869992, Arg2200Cys) (Girl et al., Nat Genet., 2019, 51, 51-62). In UKB WES, it was demonstrated that a burden of rare pLOFs and deleterious missense variants in SLC9A3R2, as well as Arg171Trp, remain highly associated with SBP, DBP and hypertension after conditioning on Arg2200Cys in PKD1 (Table 2). Overall, the signal is consistent with the well-established role of sodium balance in regulating blood pressure and suggests that blocking SLC9A3R2 could provide an attractive means for managing blood pressure.

TABLE 2

| | | | | | | |
|---|---|---|---|---|---|---|
| | | | | | N cases with | N controls with |
| | | | Effect | P- | 0\|1\|2 copies of | 0\|1\|2 copies of |
| Trait | Gene | Marker | (95% Cl) | value | effect allele | effect allele |

Original Model

| | | | | | | |
|---|---|---|---|---|---|---|
| Diastolic | SLC9A3R2 | Arg171Trp | −0.11 | 1.40E−16 | 402,124\|5,515\|14 | — |
| blood | | | (−0.13, −0.08) | | | |
| pressure | | M3.1 | −0.10 | 3.70E−18 | 400,020\|6,289\|15 | — |
| | | | (−0.13, −0.08) | | | |
| | PKD1 | Arg2200Cys | −0.04 | 7.90E−08 | 394,704\|13,396\|115 | — |
| | | | (−0.063, −0.03) | | | |
| Systolic | SLC9A3R2 | Arg171Trp | −0.10 | 8.80E−18 | 402,120\|5,515\|14 | — |
| blood | | | (−0.13, −0.08) | | | |
| pressure | | M3.1 | −0.10 | 2.00E−19 | 401,874\|6,322\|15 | — |
| | | | (−0.12, −0.08) | | | |
| Hypertension | SLC9A3R2 | Arg171Trp | 0.80 | 3.30E−10 | 103,735\|1,242\|4 | 264,855\|3,856\| |
| (ICD10\|10) | | | (0.75, 0.86) | | | 9 |
| | | M3.1 | 0.81 | 2.20E−10 | 103,677\|1,436\|4 | 264,657\|4,424\| |
| | | | (0.76, 0.87) | | | 9 |
| | PKD1 | Arg2200Cys | 0.92 | 8.80E−05 | 101,793\|3,297\|27 | 259,907\|9,113\| |
| | | | (0.88, 0.96) | | | 70 |

Conditioned on PKD1 Arg2200Cys

| | | | | | | |
|---|---|---|---|---|---|---|
| Diastolic | SLC9A3R2 | Arg171Trp | −0.1 | 2.00E−10 | 402,124\|5,515\|14 | — |
| blood | | | (−0.13,−0.07) | | | |
| pressure | | M3.1 | −0.1 | 6.20E−12 | 401,878\|6,322\|15 | — |
| | | | (−0.12, −0.07) | | | |
| Systolic | SLC9A3R2 | Arg171Trp | −0.09 | 1.00E−10 | 402,120\|5,515\|14 | — |
| blood | | | (−0.12, −0.06) | | | |
| pressure | | M3.1 | −0.09 | 2.35E−12 | 401,874\|6,322\|15 | — |
| | | | (−0.12, −0.07) | | | |
| Hypertension | SLC9A3R2 | Arg171Trp | 0.82 | 7.40E−07 | 103,735\|1,242\|4 | 264,855\|3,856\| |
| (ICD10\|10) | | | (0.75, 0.88) | | | 9 |
| | | M3.1 | 0.83 | 4.40E−07 | 103,677\|1,436\|4 | 264,657\|4,424\| |
| | | | (0.77, 0.89) | | | 9 |

Conditioned on SLC9A3R2 Arg171Trp

| | | | | | | |
|---|---|---|---|---|---|---|
| Diastolic | PKD1 | Arg2200Cys | −0.01 | 0.32 | 394,704\|13,396\|115 | |
| blood | | | (−0.03, 0.009) | | | |
| pressure | SLC9A3R2 | M3.1 | −0.09 | 7.26E−03 | 401,878\|6,322\|15 | — |
| | | | (−0.15, −0.02) | | | |
| Systolic | PKD1 | Arg2200Cys | −0.01 | 0.17 | 394,700\|13,396\|115 | — |
| blood | | | (0.03, 0.006) | | | |
| pressure | SLC9A3R2 | M3.1 | −0.09 | 3.71E−03 | 401,874\|6,322\|15 | — |
| | | | (−0.15, −0.02) | | | |
| Hypertension | PKD1 | Arg2200Cys | 0.99 | 0.57 | 101,793\|3,297\|27 | 259,907\|9,113\| |
| (ICD10\|10) | | | (0.94, 1.04) | | | 70 |
| | SLC9A3R2 | M3.1 | 0.89 | 0.19 | 103,677\|1,436\|4 | 264,657\|4,424\| |
| | | | (0.75, 1.06) | | | 9 |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having hypertension, coronary heart disease, or atrial fibrillation or at risk of developing hypertension, coronary heart disease, or atrial fibrillation, the method comprising administering a Solute Carrier Family 9 Isoform A3 Regulatory Factor 2 (SLC9A3R2) inhibitor to the subject.

2. The method according to claim 1, wherein the hypertension is secondary hypertension, resistant hypertension, or malignant hypertension.

3-7. (canceled)

8. The method according to claim 1, wherein the SLC9A3R2 inhibitor comprises an inhibitory nucleic acid molecule.

9. The method according to claim 8, wherein the inhibitory nucleic acid molecule comprises an antisense nucleic acid molecule, a small interfering RNA (siRNA), or a short hairpin RNA (shRNA) that hybridizes to an SLC9A3R2 nucleic acid molecule.

10-16. (canceled)

17. The method according to claim 1, further comprising detecting the presence or absence of an SLC9A3R2 missense variant nucleic acid molecule encoding an SLC9A3R2 predicted loss-of-function polypeptide in a biological sample obtained from the subject.

18. The method according to claim 17, further comprising administering a therapeutic agent that treats or prevents hypertension, coronary heart disease, and/or atrial fibrillation in a standard dosage amount to a subject wherein the SLC9A3R2 missense variant nucleic acid molecule is absent from the biological sample.

19. The method according to claim 17, further comprising administering a therapeutic agent that treats or prevents hypertension, coronary heart disease, and/or atrial fibrillation in a dosage amount that is the same as or less than a standard dosage amount to a subject that is heterozygous for the SLC9A3R2 missense variant nucleic acid molecule.

20. The method according to claim 17, wherein the SLC9A3R2 missense variant nucleic acid molecule encodes Arg171Trp-Long, Arg171Trp-Short, Arg65Trp, Arg58Trp, Arg60Trp-Short, Arg60Trp-Long, or Arg170Trp.

21. The method according to claim 17, wherein the SLC9A3R2 missense variant nucleic acid molecule encodes Arg171Trp-Long or Arg171Trp-Short.

22. The method according to claim 20, wherein the SLC9A3R2 missense variant nucleic acid molecule is:
a genomic nucleic acid molecule having a nucleotide sequence comprising a thymine at a position corresponding to position 9,519 according to SEQ ID NO:2, or the complement thereof;
an mRNA molecule having a nucleotide sequence comprising: a uracil at a position corresponding to position 615 according to SEQ ID NO:22, or the complement thereof; a uracil at a position corresponding to position 589 according to SEQ ID NO:23, or the complement thereof; a uracil at a position corresponding to position 353 according to SEQ ID NO:24, or the complement thereof; a uracil at a position corresponding to position 230 according to SEQ ID NO:25, or the complement thereof; a uracil at a position corresponding to position 236 according to SEQ ID NO:26, or the complement thereof; a uracil at a position corresponding to position 236 according to SEQ ID NO:27, or the complement thereof; a uracil at a position corresponding to position 604 according to SEQ ID NO:28, or the complement thereof; or a uracil at a position corresponding to position 126 according to SEQ ID NO:29, or the complement thereof; or
a cDNA molecule produced from an mRNA molecule, wherein the cDNA molecule has a nucleotide sequence comprising: a thymine at a position corresponding to position 615 according to SEQ ID NO:59, or the complement thereof; a thymine at a position corresponding to position 589 according to SEQ ID NO:60, or the complement thereof; a thymine at a position corresponding to position 353 according to SEQ ID NO:61, or the complement thereof; a thymine at a position corresponding to position 230 according to SEQ ID NO:62, or the complement thereof; a thymine at a position corresponding to position 236 according to SEQ ID NO:63, or the complement thereof; a thymine at a position corresponding to position 236 according to SEQ ID NO:64, or the complement thereof; a thymine at a position corresponding to position 604 according to SEQ ID NO:65, or the complement thereof; or a thymine at a position corresponding to position 126 according to SEQ ID NO:66, or the complement thereof.

23-37. (canceled)

38. A method of treating a subject with a therapeutic agent that treats or prevents hypertension, coronary heart disease, and/or atrial fibrillation, wherein the subject has hypertension, coronary heart disease, and/or atrial fibrillation or is at risk of developing hypertension, coronary heart disease, and/or atrial fibrillation, the method comprising:
determining whether the subject has a Solute Carrier Family 9 Isoform A3 Regulatory Factor 2 (SLC9A3R2) missense variant nucleic acid molecule encoding an SLC9A3R2 predicted loss-of-function polypeptide by:
obtaining or having obtained a biological sample from the subject; and
performing or having performed a sequence analysis on the biological sample to determine if the subject has a genotype comprising the SLC9A3R2 missense variant nucleic acid molecule encoding the SLC9A3R2 predicted loss-of-function polypeptide; and
administering or continuing to administer the therapeutic agent that treats or prevents hypertension, coronary heart disease, and/or atrial fibrillation in a standard dosage amount to a subject that is SLC9A3R2 reference, and/or administering an SLC9A3R2 inhibitor to the subject; and
administering or continuing to administer the therapeutic agent that treats or prevents hypertension, coronary heart disease, and/or atrial fibrillation in an amount that is the same as or less than a standard dosage amount to a subject that is heterozygous for the SLC9A3R2 missense variant nucleic acid molecule, and/or administering an SLC9A3R2 inhibitor to the subject;
wherein the presence of a genotype having the SLC9A3R2 missense variant nucleic acid molecule encoding the SLC9A3R2 predicted loss-of-function polypeptide indicates the subject has a decreased risk of developing hypertension, coronary heart disease, and/or atrial fibrillation.

39. The method according to claim 38, wherein the subject is SLC9A3R2 reference, and the subject is administered or continued to be administered the therapeutic agent that treats or prevents hypertension, coronary heart disease, and/or atrial fibrillation in a standard dosage amount, and is administered an SLC9A3R2 inhibitor.

40. The method according to claim 38, wherein the subject is heterozygous for an SLC9A3R2 missense variant nucleic acid molecule, and the subject is administered or continued to be administered the therapeutic agent that treats or prevents hypertension, coronary heart disease, and/or atrial fibrillation in an amount that is the same as or less than a standard dosage amount, and is administered an SLC9A3R2 inhibitor.

41. The method according to claim 38, wherein the SLC9A3R2 missense variant nucleic acid molecule encodes Arg171Trp-Long, Arg171Trp-Short, Arg65Trp, Arg58Trp, Arg60Trp-Short, Arg60Trp-Long, or Arg170Trp.

42. The method according to claim 38, wherein the SLC9A3R2 missense variant nucleic acid molecule encodes Arg171Trp-Long or Arg171Trp-Short.

43. The method according to claim 41, wherein the SLC9A3R2 missense variant nucleic acid molecule is:
a genomic nucleic acid molecule having a nucleotide sequence comprising a thymine at a position corresponding to position 9,519 according to SEQ ID NO:2;
an mRNA molecule having a nucleotide sequence comprising: a uracil at a position corresponding to position 615 according to SEQ ID NO:22, a uracil at a position corresponding to position 589 according to SEQ ID NO:23, a uracil at a position corresponding to position 353 according to SEQ ID NO:24, a uracil at a position corresponding to position 230 according to SEQ ID NO:25, a uracil at a position corresponding to position 236 according to SEQ ID NO:26, a uracil at a position corresponding to position 236 according to SEQ ID NO:27, a uracil at a position corresponding to position 604 according to SEQ ID NO:28, or a uracil at a position corresponding to position 126 according to SEQ ID NO:29; or
a cDNA molecule produced from an mRNA molecule, wherein the cDNA molecule has a nucleotide sequence comprising: a thymine at a position corresponding to position 615 according to SEQ ID NO:59, a thymine at a position corresponding to position 589 according to SEQ ID NO:60, a thymine at a position corresponding to position 353 according to SEQ ID NO:61, a thymine at a position corresponding to position 230 according to SEQ ID NO:62, a thymine at a position corresponding to position 236 according to SEQ ID NO:63, a thymine at a position corresponding to position 236 according to SEQ ID NO:64, a thymine at a position corresponding to position 604 according to SEQ ID NO:65, or a thymine at a position corresponding to position 126 according to SEQ ID NO:66.

44-58. (canceled)

59. The method according to claim 38, wherein the SLC9A3R2 inhibitor comprises an inhibitory nucleic acid molecule.

60. The method according to claim 59, wherein the inhibitory nucleic acid molecule comprises an antisense nucleic acid molecule, a small interfering RNA (siRNA), or a short hairpin RNA (shRNA) that hybridizes to an SLC9A3R2 nucleic acid molecule.

61-99. (canceled)


SEQUENCE LISTING (112 sequences)

SEQ ID NO: 1 - DNA, 12125 bp, homo sapien
gaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtgggggccagcgctcgcccccggcccgccgccccctccccgagcgcgtccccctggggcgagcaggggtcgcacgggggcccgagggggctcccgcggggaggacgcggacgttgtcgggaggcagcggcccggcgccttcggagtcccggcgcaggaagctcagtcctgcacgggggtgggggggggcatcctggcagggaggggcccgcactgcggctcctggccgctggcctcgccctttggtcgcgttttggcctcagtgtgccgtgaaggagggtgttgggccgcctggagcccctccggaaaggtgaagacctgggtgcttttgcctgggtgtgtgtgtacacgtgtgcgtgtgaacacagctgggcgtgtgtatgcaggtccctttggggtcagccatgtgtgcatgcgcctgcctgtgcgtacccaggaggggtcgcagtaaaggcatcagtgtgtctgcacctgtgtgtgctgtagtgtgtgtgtgagtatacaggtctgcacgtgtgcctgtgtgtgtgcaggtctggactcctgggcgtatgggagctgggctctgtgtgtgtgcacgcctggctagtgtactcatcactgtctgcaggccggtgagtctttgtgtctgtactctagtgtgtcccagctggtgtgtctgggaccgggctgagttggtcattgctggcaaccacattccttagctacaaaggcactactcctcctcagaggctgcctagcctggctggggttactggccgaagcctcctgcctcctggctaaggagaacagagccttttctctgtgggttctgacccctgaggtgtgtctgtctcccacaggccagggactccctccagaccctggttggggtccagcgtccacacaccccttttcctccaggtcccctgtccctcacagcaccgatgaatccataacctgtctacatggctggtcctggaacctagagattagcaggttggttcagctggacctggagctggtctcctgtttacctggtgccttgtctgtcactctgtcggccggccccatgccttccacccagagtgtctgctgtgtaaggttgtttgctgaagaatgtggggatcaggccaggggcctggacctgagggattgagggtagggctgccagggctggggccaggctgctgttggatagtcactgccaccctcgccctgggaacacctgcttttccaaggccctgtggggctggtctctgaggactggacacacagaccatggtggactctccccactgccccaggagggcaagaggggcagttactgccccatttgacagttgaggaaactgatgccagagattgagggtaacttgtctgggacacatagtcaagaagcagctgggcactgggttcctcttgtctcactctgggtcatggggagagaggagggctgggggaagatgggcctggctcagtccctcctatctgttcaccacccctgctgagcccggcagctggtggggagccttgtccaccctcccccaggccgcaggtgggagtagggctgcccctcttcctgcccacttcccagtgggcactgctttctctccgcgggtgtgcgctggccccccgcgtggtccagacacttcacaggcacctgcacgcacacaccgacggccaccaagccacagctgtccacaggtgcacctgcctctccccctcgccggctgcacctgaatcctgcttccctggctgtgtgtgggtgcccgagccaggagcccagcccttcccctctctgcagatccagagggaatgggcccatggccatggcctggaggtagatataaggcctagggtcaggagtttagactcccacactgcacggcagagaatgacgggaatgggcacagacacactcaaactggggcccagggagacacacctctccttcaaacctggacggagacacagactttgaaacagatggatgcatgcgcgcacagggacaggcagactgaaaacctgaaggtccactgtgcaccatttttgtcacttccagacaggacttacccagcggccctatcctgcagccccttcccagccccgcctggagaagaccctgggctaagggcaaggctctgggagcaccctgaagccctcacagcctgggcctcagtgtccggagcctgagtgcagagggagggagccgctggagtccgggggtggggggcggggccggcaggcctcttggtgcagccacccgcccatgcagaccagccgcctgtccgcacgcctgggccagcccagagtggtgccactgcccatgtgctgcccggaatgtgggccactgacccgctcctatcgcccattcgccccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggtatggcgggcttggcccacagggaccaccctagcctctcccagaggctctctcagcttttgacgacgatctttgcctttggtcacctccagaagtcttggtctcttctgccttttggggctccttttctgccccagggaccatcttgttggccactgggggccactgtttctaccctcagcctgtgttttaaactgggtagggtcagggcagcctggcctgttcctcttctccaggatgctccagagccacttgccctatcgaggggtcactctgacaagctgccgcctccccagcccccagcctcgtggctgaggaaccctgggaagaggcattccaggtctgcgtgtgcgcagctctgtgcctgtgctgggtgtcatccgtggctctgtctttgtctctgttgaaggcgcctgcatgctgggaaggcctgggagagggccggccttctccacaggcagcgccgtggttggggaagcctaggaggccactctgccaggcctggcgtaggtggggctgggagcatgggagtggagttgggggtccaggaggctccagggtgagcccactcctccccctagatacaagggagggccctgggaccctgtctgctgttcctcctggactcctgctgggtagtacttcctccttcccctgcccttgctcccagccaggagacccagaaaagtacaggggcggcctgctccacctcctgctaggtggccgactgggaggggcggcctggcaggccttggggaccaggaggaacagcctgaagctgtctgtcttatcttcctggaggccctgatggcattatctccatgttctctcctacctttttttttttttttttttcattttcaaagagaaccctggccgggtgcggtggctcacgcctgtaatcccaacacttcgggaggctgagatgggtggatcacctgaggtcaggaattttgagaccagcttgactaatgtggtgaaacccccgtctctactaaaaatacaaaaatcagttgggcatggtggggcacgcctatagtcccagctacttgggaggctgaggcaggagagtcgcttgaacccggcaggcggaggtttcagtgagccgagatcccgccatagcactccagcctaggcgacagagtgagactgtctcaaaaaaaaacaaaaaaaggaaaagaaagaaatcccttcaggggatatgagtccaggggttggccagaccccactgtgagaaccgcgcttggggcaggggtgtgggatctggagttctgagggtgcaggggatatacacattcactttctccctttcccagacgtcactgctgaagctggctgcccctaggagctgagcccggggggagcggcttggcagggagacagcattccctgtggggagcccagcctgggcgcaggcctgcccgtgggacctgtgggacccgttttgttcctggggcttgagggcgggccacagtggtgctcagggccaaggtgcccagcccagggctggtgggggggctggccttgcaggggggctggctggtcctaagcccctgtctccaggctgagggttggggcctccctctttcccagccaccttccctcccccaggaggaagctccccctctgcctcaggcgtttgtccattgatttggacacaaacgcatggttcaccttcctgtggaaaaatccggccctcggactttatctggagggtgcagtggcccaggcaccttgccttgggtgggaccttccccctctgctatttcaggctctgggggctgcaggaagccctaggaggggcttcctctgggtgtatttttagaggaacaaagggtggcctgagtggcaggtccctttgtgcatgagtctggctggggacaggggctccaggaggtcagggggcacccaaggaacagggtctccccttctctcttctggcatcaagacctgaggtttctttttctttttctttcttggctcactacaacctccgctgtctgggttcaagcgattctcttgcctcaccctccccagtagctgggattacaggcgcgcaccaccacacctgactaatttttgtgtttttagtagagacggggtttcaccatgttgctcaagctggtctcgaactcctgatctcaggtgatctgccctcctcgacctcccaaagttctgggattacaggtgtgagccactgtacccggcccctgaggtctgaggtttcatttctttctttctttttttttttttttgagacggagtttcactctgtcgcccaggctggagtgctgtggtgcgatctcggctcactgcaagctccgcctcctgggttcaagcaattcttatgtctcagcctgctgagtagctgggattacaggcgtgtaccaccacacctggctaatttttgtgtttttggtagagacagggtttcaccgtgttggccaggctggtctcaaactcttgggctcaagtgatccgcctgcctcagcctcccaaagtgctgggattacaggtgtgagcccctgcgcctggctcaggacctgaggtttcttcacctgagctcccagcatgggcaggaagaggtgggcctcacctgacctccacagagcaggctcagatcctgcccttctcagcctgggttcagggcatgcctcagatggtcagggagcccaggagggcaaagcttaccttgcggggatggctccagtgggggcactggtggcagagaggcagagctctggaagggatgtgtgtagtttgtctttggtgaaggatggagagggcccaactttgggtgccccctgggagctaagggtggctctcagtgtcctctgtcctggctggggataggggaggtgggcaggggaggacactgatgtggttggcccctccttttgggggccgtgaaggcctagttagtgatgactcagccctgggccacaccaggctgggggtggccgcagctgcagtgcagctgatcctagtagtgacgacaggggacagcccccaaacagagacggggtggccagcggtgccaggccctggaggtggtggcagaccctgagccctctgccccctgcccctagcccatgtctccacccccatctggagggccttgggggctaagagcaggtgtgaggggtgacagttctgcgggaggcggctgtgtggttggggcgcggtgcctgcgggggcttccgggcttccctggctcttgctcctctggagcctcaacaggaccgtcctgtgttctggggccgggactcctccctgtcccctcagccctgggtcctggggcagggcagggccttccagcagcttccttccttcctttcttgggacggaagcctagctgggtggggggcgccaggctggagccttcgcaggggagcgggctcagtcatcaccctgctccccagagtgactcagcccccacgtccccacccatccccggggagccagggccgcagagggaggtagataagtggggtggcagcctgggtcggccagagagttcaggccaccccggccggacgcctgccacttgctgtcactgtgccgctgtcatggcacgctccgggagtgccacgccacctgcccgggctccgggagcccctccacggagcccaccccagaggctggtacaggtcaggtggggtgggaggaagggagggctaggaggaaccggcagccgctcagcctcccggggtggaaggaggggactccgggaccttgatgtaagcaggctggtcgctgagcaacaatgaaggcctttctctgccagggctggtgcgtcaccatctgggggggtgcccagggtacccccatgccagcctctttgtccggacacaggcatggctgggggaccagggatggtccagctctgtgctggccgccggcagggagggagcctggagcccgtgggagcccgagggcctggcgggaggcggcaggaagtgtgtgctgagccagggcagggaggaggaatgtgagctatgaacgccgcccgagctcctcggctctctgggccccccttccccacgagcccatgtgctgccagcagcccaccctggcccctccattgcagcccctccacctgaggttttagtgctgtgcctggcgccactgggaccctgctgacaatgaaggggtttaggtttgataggcagaacttcttgccttgtagagtgaaagacggctgcttctggacagaggcagaggggtggccagaatgaccctgagaacactgaggggtccttggcctggcctcagggccccagacacccaccagatcctgcttcctgtctggtgccctgagaaaccctgccccgcaggccgctgtgcaggcgtgggaagggctgccgggccagcactgtggggctggccgcatgtgtgggctgctgtgcgtgtttgcaggtgtgtgtggacgtcttgtgtctgtggctgtgtgatgtgtctttgcggactgggcccatatcacgctcccacctcctccactgaattgggctcctgtccccacgccttccctgcgtcccaggccagcctgggggccgtgctccacctcccctcccgaactggactcctgtccccacgccttccctccgtcccaggccagcctgggggccgtgctccacctcccctcccgaactggactcctgtccccacgccttccctccgtcccaggccagcctgggggccgtgctccacctcccctcccgaactggactcctgtccccacgccttccctccgtcccaggccagcctgggggccgtgctccacctcccctcccgaactgggctcctgtccccacgccttccctccgtcccaggccagcctgggggccgtgctccacctcccctcccgaactggactcctgtccccacgccttccctccgtcccaggccagcctgggggccgtgctccacctcccctcccgaactgggctcctgtccccacgccttccctccgtcccaggccagcctgggggctgtgctccacttcccctcccgaactggactcctgtccccacgccttccctgcgtcccaggccagcctgggggccgtgctgtgactgcatccctgaggggcactttggtgggcaggtgtcagggtcagccttgaagccccacgcctgtgccaccaggtgtgtctgggggaggagtgagtctatgtggtggggccctggagattttgggggtcttgcaccctctaattcagcccatgtggcccagctgagcccctgtggggccagggagccacaggcttgtccctggagtttctggtgtgacgggctgggtggtggccatgcagagagggttcagagggtgggcaggaggcttcagatgaggcatctggcaggctggacaggagaagggtgttccagaccaagggcaggtcctgagcccaggggacagagcactagctgtgtggagaaggacccccttgctcccttggcggggcggtcccccccgctgacatggccgtttctggtccattggggtttggagcgagcttgaaggtgggtggctggaggggttgggggtgtctgaggcctggagacctgccctcctgaggtctgagcgcccccttgccatgccgccccagccctggcctggcctggcctgtggtccctcagggacacacagggaggtaggaggtgagggaagggccctggcagcccccagcccgcctgcacagtctcccctgcgcacgccctcaagcttggcgctccctggaaacctgagctgcctcctgcctgctccggccaccgccatgtttaactgagcacgggcagccgctggccaccgccgccgcaccctggcccaccccctggctgggaggtggtcattgggcctggccttgcctgcatggtcccctggcccagagccctaccgcaggatgccagaggtaacatggggcagggcctgggatggtagcggggggtccttccgggcaccaggaggccctctgtgccctgtccacactaagctcctgccccagcccctgcttgctgggcccacgggggtggggcggctcattttcctggaatgtgaaagcaaacagagccgccaccgcagccagccccacggaggcctctggagagaaaacaaaactgctggcctaggagcgcctgccccacgctctggaggagagcccggggcagggggacgcacaggcagagccctcagggacaaccgccccaggaggccaacggcgacagttcatcccacctggtgcttcctcccaccctgcctgtgcgccacgctggcctcgagccaaaggaattctcccagcaacccgggaaggcggctgggcccgtcggggaggcttctgggtttgaaaacaggctttgcccaagttcccacagctaaagctctgtcacaggcagcctgggtgcccggagtgtttgccactgagacctgggcctgcccgtggggggcacggtaccgagtttgggcagtggcggcaccaccggggagtggcctctggaggcgggaggctggagcaagagactgaccctgtccgttgggcctgcaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtaccggcccaccagggctgcggggtgccgagtgccccgcacctgtccacactggggccctgggtccttgcagggaggagggagacgctgggaacctgagctggtgcgcctcctgctgcctcggtgggcggtggctgtgatgaatatttgatgccacacctggccacgcggcgttgggggctgtctgggcccatggcggggctgatacatgctggtggcggcaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtgggccacggcccagggcacagggtgggtgcggtgtggtggctgagcagccactgacacgctgtccccacaggtcctctgccgtcacccgtcaccaatggaaccagccctgcccaggtaagagggtggggtgcccatgagacacagggtgcctctgggggtacccaggcccgacagaggcccccttctccctggagatccgtcctcgaggtgtgctagtgacacccgattttagccctgtcacctccctttaatgcccctgtgggtctcatcctgggtcgtgccacacacaagccacgtggggcccctggggtcggggccactgccttctgtcctctcctgggcaccacctggtaaggagttgccctccagtctctgcctcctcagggagctggcggtggcctcaccccaggcgttgctcacgccggccattcctgcacctgaaccccctctgcaccatgagccggccaggccacctgcctgtcactgccaggcgcccggtgcctgccccgccgcatctggagtccaccagaggcccgaggccccctgccgagtggaggagcacacactgggggggggtgtgcctctgtgcacatgtgtgcgtgtgcaggtgttgtgtgtgtctgtgaaaccacaatctgcccttggtttcccagctcaatggtggctctgcgtgctcgtcccgaagtgacctgcctggttccgacaaggacactgaggtatggatgttctccactcctgagctcacacgtggggttgctagaggccttggggtaggcgtctgtggagatgtcagcccgggcagtggggtctctgttcaggaagtcctcgtgagccccagccccagcaggcagcctctgttggttgctccctaggaggggccaccgtctggggtgtgggactagggctcactccagacaccccacccaccgtgctcaccccaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 2 - DNA, 12125 bp, homo sapien
gaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtgggggccagcgctcgcccccggcccgccgccccctccccgagcgcgtccccctggggcgagcaggggtcgcacgggggcccgagggggctcccgcggggaggacgcggacgttgtcgggaggcagcggcccggcgccttcggagtcccggcgcaggaagctcagtcctgcacgggggtgggggggggcatcctggcagggaggggcccgcactgcggctcctggccgctggcctcgccctttggtcgcgttttggcctcagtgtgccgtgaaggagggtgttgggccgcctggagcccctccggaaaggtgaagacctgggtgcttttgcctgggtgtgtgtgtacacgtgtgcgtgtgaacacagctgggcgtgtgtatgcaggtccctttggggtcagccatgtgtgcatgcgcctgcctgtgcgtacccaggaggggtcgcagtaaaggcatcagtgtgtctgcacctgtgtgtgctgtagtgtgtgtgtgagtatacaggtctgcacgtgtgcctgtgtgtgtgcaggtctggactcctgggcgtatgggagctgggctctgtgtgtgtgcacgcctggctagtgtactcatcactgtctgcaggccggtgagtctttgtgtctgtactctagtgtgtcccagctggtgtgtctgggaccgggctgagttggtcattgctggcaaccacattccttagctacaaaggcactactcctcctcagaggctgcctagcctggctggggttactggccgaagcctcctgcctcctggctaaggagaacagagccttttctctgtgggttctgacccctgaggtgtgtctgtctcccacaggccagggactccctccagaccctggttggggtccagcgtccacacaccccttttcctccaggtcccctgtccctcacagcaccgatgaatccataacctgtctacatggctggtcctggaacctagagattagcaggttggttcagctggacctggagctggtctcctgtttacctggtgccttgtctgtcactctgtcggccggccccatgccttccacccagagtgtctgctgtgtaaggttgtttgctgaagaatgtggggatcaggccaggggcctggacctgagggattgagggtagggctgccagggctggggccaggctgctgttggatagtcactgccaccctcgccctgggaacacctgcttttccaaggccctgtggggctggtctctgaggactggacacacagaccatggtggactctccccactgccccaggagggcaagaggggcagttactgccccatttgacagttgaggaaactgatgccagagattgagggtaacttgtctgggacacatagtcaagaagcagctgggcactgggttcctcttgtctcactctgggtcatggggagagaggagggctgggggaagatgggcctggctcagtccctcctatctgttcaccacccctgctgagcccggcagctggtggggagccttgtccaccctcccccaggccgcaggtgggagtagggctgcccctcttcctgcccacttcccagtgggcactgctttctctccgcgggtgtgcgctggccccccgcgtggtccagacacttcacaggcacctgcacgcacacaccgacggccaccaagccacagctgtccacaggtgcacctgcctctccccctcgccggctgcacctgaatcctgcttccctggctgtgtgtgggtgcccgagccaggagcccagcccttcccctctctgcagatccagagggaatgggcccatggccatggcctggaggtagatataaggcctagggtcaggagtttagactcccacactgcacggcagagaatgacgggaatgggcacagacacactcaaactggggcccagggagacacacctctccttcaaacctggacggagacacagactttgaaacagatggatgcatgcgcgcacagggacaggcagactgaaaacctgaaggtccactgtgcaccatttttgtcacttccagacaggacttacccagcggccctatcctgcagccccttcccagccccgcctggagaagaccctgggctaagggcaaggctctgggagcaccctgaagccctcacagcctgggcctcagtgtccggagcctgagtgcagagggagggagccgctggagtccgggggtggggggcggggccggcaggcctcttggtgcagccacccgcccatgcagaccagccgcctgtccgcacgcctgggccagcccagagtggtgccactgcccatgtgctgcccggaatgtgggccactgacccgctcctatcgcccattcgccccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggtatggcgggcttggcccacagggaccaccctagcctctcccagaggctctctcagcttttgacgacgatctttgcctttggtcacctccagaagtcttggtctcttctgccttttggggctccttttctgccccagggaccatcttgttggccactgggggccactgtttctaccctcagcctgtgttttaaactgggtagggtcagggcagcctggcctgttcctcttctccaggatgctccagagccacttgccctatcgaggggtcactctgacaagctgccgcctccccagcccccagcctcgtggctgaggaaccctgggaagaggcattccaggtctgcgtgtgcgcagctctgtgcctgtgctgggtgtcatccgtggctctgtctttgtctctgttgaaggcgcctgcatgctgggaaggcctgggagagggccggccttctccacaggcagcgccgtggttggggaagcctaggaggccactctgccaggcctggcgtaggtggggctgggagcatgggagtggagttgggggtccaggaggctccagggtgagcccactcctccccctagatacaagggagggccctgggaccctgtctgctgttcctcctggactcctgctgggtagtacttcctccttcccctgcccttgctcccagccaggagacccagaaaagtacaggggcggcctgctccacctcctgctaggtggccgactgggaggggcggcctggcaggccttggggaccaggaggaacagcctgaagctgtctgtcttatcttcctggaggccctgatggcattatctccatgttctctcctacctttttttttttttttttttcattttcaaagagaaccctggccgggtgcggtggctcacgcctgtaatcccaacacttcgggaggctgagatgggtggatcacctgaggtcaggaattttgagaccagcttgactaatgtggtgaaacccccgtctctactaaaaatacaaaaatcagttgggcatggtggggcacgcctatagtcccagctacttgggaggctgaggcaggagagtcgcttgaacccggcaggcggaggtttcagtgagccgagatcccgccatagcactccagcctaggcgacagagtgagactgtctcaaaaaaaaacaaaaaaaggaaaagaaagaaatcccttcaggggatatgagtccaggggttggccagaccccactgtgagaaccgcgcttggggcaggggtgtgggatctggagttctgagggtgcaggggatatacacattcactttctccctttcccagacgtcactgctgaagctggctgcccctaggagctgagcccggggggagcggcttggcagggagacagcattccctgtggggagcccagcctgggcgcaggcctgcccgtgggacctgtgggacccgttttgttcctggggcttgagggcgggccacagtggtgctcagggccaaggtgcccagcccagggctggtgggggggctggccttgcaggggggctggctggtcctaagcccctgtctccaggctgagggttggggcctccctctttcccagccaccttccctcccccaggaggaagctccccctctgcctcaggcgtttgtccattgatttggacacaaacgcatggttcaccttcctgtggaaaaatccggccctcggactttatctggagggtgcagtggcccaggcaccttgccttgggtgggaccttccccctctgctatttcaggctctgggggctgcaggaagccctaggaggggcttcctctgggtgtatttttagaggaacaaagggtggcctgagtggcaggtccctttgtgcatgagtctggctggggacaggggctccaggaggtcagggggcacccaaggaacagggtctccccttctctcttctggcatcaagacctgaggtttctttttctttttctttcttggctcactacaacctccgctgtctgggttcaagcgattctcttgcctcaccctccccagtagctgggattacaggcgcgcaccaccacacctgactaatttttgtgtttttagtagagacggggtttcaccatgttgctcaagctggtctcgaactcctgatctcaggtgatctgccctcctcgacctcccaaagttctgggattacaggtgtgagccactgtacccggcccctgaggtctgaggtttcatttctttctttctttttttttttttttgagacggagtttcactctgtcgcccaggctggagtgctgtggtgcgatctcggctcactgcaagctccgcctcctgggttcaagcaattcttatgtctcagcctgctgagtagctgggattacaggcgtgtaccaccacacctggctaatttttgtgtttttggtagagacagggtttcaccgtgttggccaggctggtctcaaactcttgggctcaagtgatccgcctgcctcagcctcccaaagtgctgggattacaggtgtgagcccctgcgcctggctcaggacctgaggtttcttcacctgagctcccagcatgggcaggaagaggtgggcctcacctgacctccacagagcaggctcagatcctgcccttctcagcctgggttcagggcatgcctcagatggtcagggagcccaggagggcaaagcttaccttgcggggatggctccagtgggggcactggtggcagagaggcagagctctggaagggatgtgtgtagtttgtctttggtgaaggatggagagggcccaactttgggtgccccctgggagctaagggtggctctcagtgtcctctgtcctggctggggataggggaggtgggcaggggaggacactgatgtggttggcccctccttttgggggccgtgaaggcctagttagtgatgactcagccctgggccacaccaggctgggggtggccgcagctgcagtgcagctgatcctagtagtgacgacaggggacagcccccaaacagagacggggtggccagcggtgccaggccctggaggtggtggcagaccctgagccctctgccccctgcccctagcccatgtctccacccccatctggagggccttgggggctaagagcaggtgtgaggggtgacagttctgcgggaggcggctgtgtggttggggcgcggtgcctgcgggggcttccgggcttccctggctcttgctcctctggagcctcaacaggaccgtcctgtgttctggggccgggactcctccctgtcccctcagccctgggtcctggggcagggcagggccttccagcagcttccttccttcctttcttgggacggaagcctagctgggtggggggcgccaggctggagccttcgcaggggagcgggctcagtcatcaccctgctccccagagtgactcagcccccacgtccccacccatccccggggagccagggccgcagagggaggtagataagtggggtggcagcctgggtcggccagagagttcaggccaccccggccggacgcctgccacttgctgtcactgtgccgctgtcatggcacgctccgggagtgccacgccacctgcccgggctccgggagcccctccacggagcccaccccagaggctggtacaggtcaggtggggtgggaggaagggagggctaggaggaaccggcagccgctcagcctcccggggtggaaggaggggactccgggaccttgatgtaagcaggctggtcgctgagcaacaatgaaggcctttctctgccagggctggtgcgtcaccatctgggggggtgcccagggtacccccatgccagcctctttgtccggacacaggcatggctgggggaccagggatggtccagctctgtgctggccgccggcagggagggagcctggagcccgtgggagcccgagggcctggcgggaggcggcaggaagtgtgtgctgagccagggcagggaggaggaatgtgagctatgaacgccgcccgagctcctcggctctctgggccccccttccccacgagcccatgtgctgccagcagcccaccctggcccctccattgcagcccctccacctgaggttttagtgctgtgcctggcgccactgggaccctgctgacaatgaaggggtttaggtttgataggcagaacttcttgccttgtagagtgaaagacggctgcttctggacagaggcagaggggtggccagaatgaccctgagaacactgaggggtccttggcctggcctcagggccccagacacccaccagatcctgcttcctgtctggtgccctgagaaaccctgccccgcaggccgctgtgcaggcgtgggaagggctgccgggccagcactgtggggctggccgcatgtgtgggctgctgtgcgtgtttgcaggtgtgtgtggacgtcttgtgtctgtggctgtgtgatgtgtctttgcggactgggcccatatcacgctcccacctcctccactgaattgggctcctgtccccacgccttccctgcgtcccaggccagcctgggggccgtgctccacctcccctcccgaactggactcctgtccccacgccttccctccgtcccaggccagcctgggggccgtgctccacctcccctcccgaactggactcctgtccccacgccttccctccgtcccaggccagcctgggggccgtgctccacctcccctcccgaactggactcctgtccccacgccttccctccgtcccaggccagcctgggggccgtgctccacctcccctcccgaactgggctcctgtccccacgccttccctccgtcccaggccagcctgggggccgtgctccacctcccctcccgaactggactcctgtccccacgccttccctccgtcccaggccagcctgggggccgtgctccacctcccctcccgaactgggctcctgtccccacgccttccctccgtcccaggccagcctgggggctgtgctccacttcccctcccgaactggactcctgtccccacgccttccctgcgtcccaggccagcctgggggccgtgctgtgactgcatccctgaggggcactttggtgggcaggtgtcagggtcagccttgaagccccacgcctgtgccaccaggtgtgtctgggggaggagtgagtctatgtggtggggccctggagattttgggggtcttgcaccctctaattcagcccatgtggcccagctgagcccctgtggggccagggagccacaggcttgtccctggagtttctggtgtgacgggctgggtggtggccatgcagagagggttcagagggtgggcaggaggcttcagatgaggcatctggcaggctggacaggagaagggtgttccagaccaagggcaggtcctgagcccaggggacagagcactagctgtgtggagaaggacccccttgctcccttggcggggcggtcccccccgctgacatggccgtttctggtccattggggtttggagcgagcttgaaggtgggtggctggaggggttgggggtgtctgaggcctggagacctgccctcctgaggtctgagcgcccccttgccatgccgccccagccctggcctggcctggcctgtggtccctcagggacacacagggaggtaggaggtgagggaagggccctggcagcccccagcccgcctgcacagtctcccctgcgcacgccctcaagcttggcgctccctggaaacctgagctgcctcctgcctgctccggccaccgccatgtttaactgagcacgggcagccgctggccaccgccgccgcaccctggcccaccccctggctgggaggtggtcattgggcctggccttgcctgcatggtcccctggcccagagccctaccgcaggatgccagaggtaacatggggcagggcctgggatggtagcggggggtccttccgggcaccaggaggccctctgtgccctgtccacactaagctcctgccccagcccctgcttgctgggcccacgggggtggggcggctcattttcctggaatgtgaaagcaaacagagccgccaccgcagccagccccacggaggcctctggagagaaaacaaaactgctggcctaggagcgcctgccccacgctctggaggagagcccggggcagggggacgcacaggcagagccctcagggacaaccgccccaggaggccaacggcgacagttcatcccacctggtgcttcctcccaccctgcctgtgcgccacgctggcctcgagccaaaggaattctcccagcaacccgggaaggcggctgggcccgtcggggaggcttctgggtttgaaaacaggctttgcccaagttcccacagctaaagctctgtcacaggcagcctgggtgcccggagtgtttgccactgagacctgggcctgcccgtggggggcacggtaccgagtttgggcagtggcggcaccaccggggagtggcctctggaggcgggaggctggagcaagagactgaccctgtccgttgggcctgcaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtaccggcccaccagggctgcggggtgccgagtgccccgcacctgtccacactggggccctgggtccttgcagggaggagggagacgctgggaacctgagctggtgcgcctcctgctgcctcggtgggcggtggctgtgatgaatatttgatgccacacctggccacgcggcgttgggggctgtctgggcccatggcggggctgatacatgctggtggcggcaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtgggccacggcccagggcacagggtgggtgcggtgtggtggctgagcagccactgacacgctgtccccacaggtcctctgccgtcacccgtcaccaatggaaccagccctgcccaggtaagagggtggggtgcccatgagacacagggtgcctctgggggtacccaggcccgacagaggcccccttctccctggagatccgtcctcgaggtgtgctagtgacacccgattttagccctgtcacctccctttaatgcccctgtgggtctcatcctgggtcgtgccacacacaagccacgtggggcccctggggtcggggccactgccttctgtcctctcctgggcaccacctggtaaggagttgccctccagtctctgcctcctcagggagctggcggtggcctcaccccaggcgttgctcacgccggccattcctgcacctgaaccccctctgcaccatgagccggccaggccacctgcctgtcactgccaggcgcccggtgcctgccccgccgcatctggagtccaccagaggcccgaggccccctgccgagtggaggagcacacactgggggggggtgtgcctctgtgcacatgtgtgcgtgtgcaggtgttgtgtgtgtctgtgaaaccacaatctgcccttggtttcccagctcaatggtggctctgcgtgctcgtcccgaagtgacctgcctggttccgacaaggacactgaggtatggatgttctccactcctgagctcacacgtggggttgctagaggccttggggtaggcgtctgtggagatgtcagcccgggcagtggggtctctgttcaggaagtcctcgtgagccccagccccagcaggcagcctctgttggttgctccctaggaggggccaccgtctggggtgtgggactagggctcactccagacaccccacccaccgtgctcaccccaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 3 - RNA, 2160 nt, homo sapien
gaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 4 - RNA, 1564 nt, homo sapien
gccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuc

SEQ ID NO: 5 - RNA, 1037 nt, homo sapien
augugcugcccggaaugugggccacugacccgcuccuaucgcccauucgccccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccugugg

SEQ ID NO: 6 - RNA, 1775 nt, homo sapien
agagaguucaggccaccccggccggacgccugccacuugcugucacugugccgcugucauggcacgcuccgggagugccacgccaccugcccgggcuccgggagccccuccacggagcccaccccagaggcuggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 7 - RNA, 1748 nt, homo sapien
agagaguucaggccaccccggccggacgccugccacuugcugucacugugccgcugucauggcacgcuccgggagugccacgccaccugcccgggcuccgggagccccuccacggagcccaccccagaggcugguacaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 8 - RNA, 1781 nt, homo sapien
agagaguucaggccaccccggccggacgccugccacuugcugucacugugccgcugucauggcacgcuccgggagugccacgccaccugcccgggcuccgggagccccuccacggagcccaccccagaggcugguacaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 9 - RNA, 1600 nt, homo sapien
gccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcuggcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucaucccgaagugaccugccugguuccgacaaggacacugaggauggcagugccuggaagcaagaucccuuccaggagagcggccuccaccugagccccacggcggccgaggcaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccuuccccucccccuuccccucccccuugggacgcgcucuaaauaauugcaauaaaacaaaccuuucucugc

SEQ ID NO: 10 - RNA, 738 nt, homo sapien
gcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggaggggccaccgucuggggugugggacuagggcucacuccagacaccccacccaccgugcucaccccaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccg

SEQ ID NO: 11 - RNA, 578 nt, homo sapien
agagagauggaagcggccggccgggcgggagccugaagcagccuuacccaggaauugcagggguugggguggcgagcagucgcggcgccucagccuaaguggcuuuuaggggcagcugccgggauaugggcacccagggggccauggcccaggaggcugcccagaggcgagcccugugcuugggugggggcaggggagcccagcccagggccccggagagacgagagggagaggaggacugcgccaagccugcuucuacggcucacauggggaauugccugggcuggcugugccugaccccugggaaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggc

SEQ ID NO: 12 - RNA, 1628 nt, homo sapien
gacgggagccgaacaugagccgccgcugaagccaccgcggggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccccuuccccucccccuugggacgcgcucuaaauaauugcaauaaaacaaaccuuucucugcaaaaaaaaaaaaaaaa

SEQ ID NO: 13 - RNA, 1639 nt, homo sapien
gggagccgaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccccuuccccucccccuuccccucccccuugggacgcgcucuaaauaauugcaauaaaacaaaccuuucucugcaaaaaaaaaaaaaaaaaa

SEQ ID NO: 14 - RNA, 1622 nt, homo sapien
gccgaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuugggacgcgcucuaaauaauugcaauaaaacaaaccuuucucugcaaaaaaaaaaaaaaaaaa

SEQ ID NO: 15 - RNA, 1524 nt, homo sapien
gugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucaucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccuuccccucccccuuccccucccccuugggacgcgcucuaaauaauugcaauaaaacaaaccuuucucugc

SEQ ID NO: 16 - RNA, 1357 nt, homo sapien
auggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacuga

SEQ ID NO: 17 - RNA, 2194 nt, homo sapien
ggagcccgagccggaucccgaggcgacgggagccgaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 18 - RNA, 2127 nt, homo sapien
gaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 19 - RNA, 1578 nt, homo sapien
gaauucggcacgagccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccuuccccucccccuuccccucccccuugggacgcgcucuaaauaauugcaauaaaacaaaccuuucucugcaaaaaggaauuc

SEQ ID NO: 20 - RNA, 759 nt, homo sapien
agauaagugggguggcagccugggucggccagagaguucaggccaccccggccggacgccugccacuugcugucacugugccgcugucauggcacgcuccgggagugccacgccaccugcccgggcuccgggagccccuccacggagcccaccccagaggcuggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuu

SEQ ID NO: 21 - RNA, 1199 nt, homo sapien
ugggguggcagccugggucggccagagaguucaggccaccccggccggacgccugccacuugcugucacugugccgcugucauggcacgcuccgggagugccacgccaccugcccgggcuccgggagccccuccacggagcccaccccagaggcugguacaggaugucagugggccccugagggagcugcgcccucggcucugccaccugcgaaagggaccucagggcuauggguucaaccugcauagugacaagucccggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccuccccc

SEQ ID NO: 22 - RNA, 2160 nt, homo sapien
gaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 23 - RNA, 1564 nt, homo sapien
gccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuc

SEQ ID NO: 24 - RNA, 1037 nt, homo sapien
augugcugcccggaaugugggccacugacccgcuccuaucgcccauucgccccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccugugg

SEQ ID NO: 25 - RNA, 1775 nt, homo sapien
agagaguucaggccaccccggccggacgccugccacuugcugucacugugccgcugucauggcacgcuccgggagugccacgccaccugcccgggcuccgggagccccuccacggagcccaccccagaggcuggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 26 - RNA, 1748 nt, homo sapien
agagaguucaggccaccccggccggacgccugccacuugcugucacugugccgcugucauggcacgcuccgggagugccacgccaccugcccgggcuccgggagccccuccacggagcccaccccagaggcugguacaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 27 - RNA, 1781 nt, homo sapien
agagaguucaggccaccccggccggacgccugccacuugcugucacugugccgcugucauggcacgcuccgggagugccacgccaccugcccgggcuccgggagccccuccacggagcccaccccagaggcugguacaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 28 - RNA, 1600 nt, homo sapien
gccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcuggcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucaucccgaagugaccugccugguuccgacaaggacacugaggauggcagugccuggaagcaagaucccuuccaggagagcggccuccaccugagccccacggcggccgaggcaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccuuccccucccccuuccccucccccuugggacgcgcucuaaauaauugcaauaaaacaaaccuuucucugc

SEQ ID NO: 29 - RNA, 738 nt, homo sapien
gcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggaggggccaccgucuggggugugggacuagggcucacuccagacaccccacccaccgugcucaccccaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccg

SEQ ID NO: 30 - RNA, 1628 nt, homo sapien
gacgggagccgaacaugagccgccgcugaagccaccgcggggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccccuuccccucccccuugggacgcgcucuaaauaauugcaauaaaacaaaccuuucucugcaaaaaaaaaaaaaaaa

SEQ ID NO: 31 - RNA, 1639 nt, homo sapien
gggagccgaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccccuuccccucccccuuccccucccccuugggacgcgcucuaaauaauugcaauaaaacaaaccuuucucugcaaaaaaaaaaaaaaaaaa

SEQ ID NO: 32 - RNA, 1622 nt, homo sapien
gccgaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuugggacgcgcucuaaauaauugcaauaaaacaaaccuuucucugcaaaaaaaaaaaaaaaaaa

SEQ ID NO: 33 - RNA, 1524 nt, homo sapien
gugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucaucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccuuccccucccccuuccccucccccuugggacgcgcucuaaauaauugcaauaaaacaaaccuuucucugc

SEQ ID NO: 34 - RNA, 1357 nt, homo sapien
auggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacuga

SEQ ID NO: 35 - RNA, 2194 nt, homo sapien
ggagcccgagccggaucccgaggcgacgggagccgaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 36 - RNA, 2127 nt, homo sapien
gaacaggagccgccgcugaagccaccgccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccucccccuuccccucccccuugggacgcgcucuaaauaauugcaauaaaacaaaccuuucucugcaaaccauuuccuccccgcccccuccccucagcagcggccguccugagugggagucccugggacuucccaguggccaaguuggggcgcccagccucuucguggggaccuuggguaaggccagggaggccugauguggccguaggagcugccccugcccaccugcccuggugugggggucccuaggccacacccugcuccccacccagcuacccugugcgccugugcccugcugggggccugggcucuccgaggggccugaggauggaggccccacguccccgaggagggcggccucuggacaggccccucauuccgcgcggcagcucccaggccuggggaacguaggugugugagagcggcacccgggaaggacgccuggccucuggcucagcccugcuuggcgggcucccccguggacacccuguugacuuugcacuucccucccgggccccgcacccccgaaccgaccaccgaucgaccggcaccgcuguugccucguaagccauagcgcaugcgcgcucucaggauaaacaggcccugccuggga

SEQ ID NO: 37 - RNA, 1578 nt, homo sapien
gaauucggcacgagccgggugcccagcgccgccgccgcccccgagcucccccgcgccccugcccgcgggcggccggugggcagcgggcgccauggccgcgccggagccgcugcggccgcgccugugccgcuuggugcgcggagagcagggcuacggcuuccaccugcacggcgagaagggccgccgcgggcaguucauccggcgcguggaacccgguucccccgccgaggccgccgcgcugcgcgcuggggaccgccuggucgaggucaacggcgucaacguggagggcgagacgcaccaccagguggugcaaaggaucaaggcuguggaggggcagacucggcugcuggugguggaccaggagacagaugaggagcuccgccggcggcagcugaccuguaccgaggagauggcccagcgagggcucccacccgcccacgaccccugggagccgaagccagacugggcacacaccggcagccacagcuccgaagcuggcaagaaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagaccccccccuuccccucccccuuccccucccccuugggacgcgcucuaaauaauugcaauaaaacaaaccuuucucugcaaaaaggaauuc

SEQ ID NO: 38 - RNA, 759 nt, homo sapien
agauaagugggguggcagccugggucggccagagaguucaggccaccccggccggacgccugccacuugcugucacugugccgcugucauggcacgcuccgggagugccacgccaccugcccgggcuccgggagccccuccacggagcccaccccagaggcuggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuu

SEQ ID NO: 39 - RNA, 1199 nt, homo sapien
ugggguggcagccugggucggccagagaguucaggccaccccggccggacgccugccacuugcugucacugugccgcugucauggcacgcuccgggagugccacgccaccugcccgggcuccgggagccccuccacggagcccaccccagaggcugguacaggaugucagugggccccugagggagcugcgcccucggcucugccaccugcgaaagggaccucagggcuauggguucaaccugcauagugacaaguccuggcccggccaguacauccgcucuguggacccgggcucaccugccgcccgcucuggccuccgcgcccaggaccggcucauugaggugaacgggcagaauguggagggacugcgccaugcugaggugguggccagcaucaaggcacgggaggacgaggcccggcugcuggucguggaccccgagacagaugaacacuucaagcggcuucgggucacacccaccgaggagcacguggaagguccucugccgucacccgucaccaauggaaccagcccugcccagcucaaugguggcucugcgugcucgucccgaagugaccugccugguuccgacaaggacacugaggauggcagugccuggaagcaagaucccuuccaggagagcggccuccaccugagccccacggcggccgaggccaaggagaaggcucgagccaugcgagucaacaagcgcgcgccacagauggacuggaacaggaagcgugaaaucuucagcaacuucugagccccuuccugccugucucgggacccugggaccccucccgcacggaccuugggccucagccugccccgagcucccccagccucaguggacuggagggugguccugccauugcccagaaaucagccccagccccggugagcccccauccugccccugcccaccagguacugggggccuguggcagcaagauagggggagagagacccagagaugugagagagagucagagacagagacagagagagagagagagagacacagagagagacagagagagagcgagcgagcgcgcggcagccgcggggcgagggccuuugcugcucugccggggccugcugacugaaaggaauuuguguuuuugcuuuuuuuccaaaaagaucuccagcuccacacauguuuccacuuaauaccagagacccccccccuuccccuccccc

SEQ ID NO: 40 - DNA, 2160 bp, homo sapien
gaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 41 - DNA, 1564 bp, homo sapien
gccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttc

SEQ ID NO: 42 - DNA, 1037 bp, homo sapien
atgtgctgcccggaatgtgggccactgacccgctcctatcgcccattcgccccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtgg

SEQ ID NO: 43 - DNA, 1775 bp, homo sapien
agagagttcaggccaccccggccggacgcctgccacttgctgtcactgtgccgctgtcatggcacgctccgggagtgccacgccacctgcccgggctccgggagcccctccacggagcccaccccagaggctggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 44 - DNA, 1748 bp, homo sapien
agagagttcaggccaccccggccggacgcctgccacttgctgtcactgtgccgctgtcatggcacgctccgggagtgccacgccacctgcccgggctccgggagcccctccacggagcccaccccagaggctggtacaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 45 - DNA, 1781 bp, homo sapien
agagagttcaggccaccccggccggacgcctgccacttgctgtcactgtgccgctgtcatggcacgctccgggagtgccacgccacctgcccgggctccgggagcccctccacggagcccaccccagaggctggtacaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 46 - DNA, 1600 bp, homo sapien
gccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctggctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcatcccgaagtgacctgcctggttccgacaaggacactgaggatggcagtgcctggaagcaagatcccttccaggagagcggcctccacctgagccccacggcggccgaggcaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccttcccctcccccttcccctcccccttgggacgcgctctaaataattgcaataaaacaaacctttctctgc

SEQ ID NO: 47 - DNA, 738 bp, homo sapien
gcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggaggggccaccgtctggggtgtgggactagggctcactccagacaccccacccaccgtgctcaccccaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccg

SEQ ID NO: 48 - DNA, 578 bp, homo sapien
agagagatggaagcggccggccgggcgggagcctgaagcagccttacccaggaattgcaggggttggggtggcgagcagtcgcggcgcctcagcctaagtggcttttaggggcagctgccgggatatgggcacccagggggccatggcccaggaggctgcccagaggcgagccctgtgcttgggtgggggcaggggagcccagcccagggccccggagagacgagagggagaggaggactgcgccaagcctgcttctacggctcacatggggaattgcctgggctggctgtgcctgacccctgggaaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggc

SEQ ID NO: 49 - DNA, 1628 bp, homo sapien
gacgggagccgaacatgagccgccgctgaagccaccgcggggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccccttcccctcccccttgggacgcgctctaaataattgcaataaaacaaacctttctctgcaaaaaaaaaaaaaaaa

SEQ ID NO: 50 - DNA, 1639 bp, homo sapien
gggagccgaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccccttcccctcccccttcccctcccccttgggacgcgctctaaataattgcaataaaacaaacctttctctgcaaaaaaaaaaaaaaaaaa

SEQ ID NO: 51 - DNA, 1622 bp, homo sapien
gccgaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttgggacgcgctctaaataattgcaataaaacaaacctttctctgcaaaaaaaaaaaaaaaaaa

SEQ ID NO: 52 - DNA, 1524 bp, homo sapien
gtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcatcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccttcccctcccccttcccctcccccttgggacgcgctctaaataattgcaataaaacaaacctttctctgc

SEQ ID NO: 53 - DNA, 1357 bp, homo sapien
atggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactga

SEQ ID NO: 54 - DNA, 2194 bp, homo sapien
ggagcccgagccggatcccgaggcgacgggagccgaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 55 - DNA, 2127 bp, homo sapien
gaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 56 - DNA, 1578 bp, homo sapien
gaattcggcacgagccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccttcccctcccccttcccctcccccttgggacgcgctctaaataattgcaataaaacaaacctttctctgcaaaaaggaattc

SEQ ID NO: 57 - DNA, 759 bp, homo sapien
agataagtggggtggcagcctgggtcggccagagagttcaggccaccccggccggacgcctgccacttgctgtcactgtgccgctgtcatggcacgctccgggagtgccacgccacctgcccgggctccgggagcccctccacggagcccaccccagaggctggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaactt

SEQ ID NO: 58 - DNA, 1199 bp, homo sapien
tggggtggcagcctgggtcggccagagagttcaggccaccccggccggacgcctgccacttgctgtcactgtgccgctgtcatggcacgctccgggagtgccacgccacctgcccgggctccgggagcccctccacggagcccaccccagaggctggtacaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctccccc

SEQ ID NO: 59 - DNA, 2160 bp, homo sapien
gaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 60 - DNA, 1564 bp, homo sapien
gccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttc

SEQ ID NO: 61 - DNA, 1037 bp, homo sapien
atgtgctgcccggaatgtgggccactgacccgctcctatcgcccattcgccccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtgg

SEQ ID NO: 62 - DNA, 1775 bp, homo sapien
agagagttcaggccaccccggccggacgcctgccacttgctgtcactgtgccgctgtcatggcacgctccgggagtgccacgccacctgcccgggctccgggagcccctccacggagcccaccccagaggctggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 63 - DNA, 1748 bp, homo sapien
agagagttcaggccaccccggccggacgcctgccacttgctgtcactgtgccgctgtcatggcacgctccgggagtgccacgccacctgcccgggctccgggagcccctccacggagcccaccccagaggctggtacaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 64 - DNA, 1781 bp, homo sapien
agagagttcaggccaccccggccggacgcctgccacttgctgtcactgtgccgctgtcatggcacgctccgggagtgccacgccacctgcccgggctccgggagcccctccacggagcccaccccagaggctggtacaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 65 - DNA, 1600 bp, homo sapien
gccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctggctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcatcccgaagtgacctgcctggttccgacaaggacactgaggatggcagtgcctggaagcaagatcccttccaggagagcggcctccacctgagccccacggcggccgaggcaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccttcccctcccccttcccctcccccttgggacgcgctctaaataattgcaataaaacaaacctttctctgc

SEQ ID NO: 66 - DNA, 738 bp, homo sapien
gcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggaggggccaccgtctggggtgtgggactagggctcactccagacaccccacccaccgtgctcaccccaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccg

SEQ ID NO: 67 - DNA, 1628 bp, homo sapien
gacgggagccgaacatgagccgccgctgaagccaccgcggggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccccttcccctcccccttgggacgcgctctaaataattgcaataaaacaaacctttctctgcaaaaaaaaaaaaaaaa

SEQ ID NO: 68 - DNA, 1639 bp, homo sapien
gggagccgaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccccttcccctcccccttcccctcccccttgggacgcgctctaaataattgcaataaaacaaacctttctctgcaaaaaaaaaaaaaaaaaa

SEQ ID NO: 69 - DNA, 1622 bp, homo sapien
gccgaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttgggacgcgctctaaataattgcaataaaacaaacctttctctgcaaaaaaaaaaaaaaaaaa

SEQ ID NO: 70 - DNA, 1524 bp, homo sapien
gtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcatcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccttcccctcccccttcccctcccccttgggacgcgctctaaataattgcaataaaacaaacctttctctgc

SEQ ID NO: 71 - DNA, 1357 bp, homo sapien
atggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactga

SEQ ID NO: 72 - DNA, 2194 bp, homo sapien
ggagcccgagccggatcccgaggcgacgggagccgaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 73 - DNA, 2127 bp, homo sapien
gaacaggagccgccgctgaagccaccgccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctcccccttcccctcccccttgggacgcgctctaaataattgcaataaaacaaacctttctctgcaaaccatttcctccccgccccctcccctcagcagcggccgtcctgagtgggagtccctgggacttcccagtggccaagttggggcgcccagcctcttcgtggggaccttgggtaaggccagggaggcctgatgtggccgtaggagctgcccctgcccacctgccctggtgtgggggtccctaggccacaccctgctccccacccagctaccctgtgcgcctgtgccctgctgggggcctgggctctccgaggggcctgaggatggaggccccacgtccccgaggagggcggcctctggacaggcccctcattccgcgcggcagctcccaggcctggggaacgtaggtgtgtgagagcggcacccgggaaggacgcctggcctctggctcagccctgcttggcgggctcccccgtggacaccctgttgactttgcacttccctcccgggccccgcacccccgaaccgaccaccgatcgaccggcaccgctgttgcctcgtaagccatagcgcatgcgcgctctcaggataaacaggccctgcctggga

SEQ ID NO: 74 - DNA, 1578 bp, homo sapien
gaattcggcacgagccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagaccccccccttcccctcccccttcccctcccccttgggacgcgctctaaataattgcaataaaacaaacctttctctgcaaaaaggaattc

SEQ ID NO: 75 - DNA, 759 bp, homo sapien
agataagtggggtggcagcctgggtcggccagagagttcaggccaccccggccggacgcctgccacttgctgtcactgtgccgctgtcatggcacgctccgggagtgccacgccacctgcccgggctccgggagcccctccacggagcccaccccagaggctggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaactt

SEQ ID NO: 76 - DNA, 1199 bp, homo sapien
tggggtggcagcctgggtcggccagagagttcaggccaccccggccggacgcctgccacttgctgtcactgtgccgctgtcatggcacgctccgggagtgccacgccacctgcccgggctccgggagcccctccacggagcccaccccagaggctggtacaggatgtcagtgggcccctgagggagctgcgccctcggctctgccacctgcgaaagggacctcagggctatgggttcaacctgcatagtgacaagtcctggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggatggcagtgcctggaagcaagatcccttccaggagagcggcctccacctgagccccacggcggccgaggccaaggagaaggctcgagccatgcgagtcaacaagcgcgcgccacagatggactggaacaggaagcgtgaaatcttcagcaacttctgagccccttcctgcctgtctcgggaccctgggacccctcccgcacggaccttgggcctcagcctgccccgagctcccccagcctcagtggactggagggtggtcctgccattgcccagaaatcagccccagccccggtgagcccccatcctgcccctgcccaccaggtactgggggcctgtggcagcaagatagggggagagagacccagagatgtgagagagagtcagagacagagacagagagagagagagagagacacagagagagacagagagagagcgagcgagcgcgcggcagccgcggggcgagggcctttgctgctctgccggggcctgctgactgaaaggaatttgtgtttttgctttttttccaaaaagatctccagctccacacatgtttccacttaataccagagacccccccccttcccctccccc

SEQ ID NO: 77 - protein, 337 aa, homo sapien
MAAPEPLRPRLCRLVRGEQGYGFHLHGEKGRRGQFIRRVEPGSPAEAAALRAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 78 - protein, 326 aa, homo sapien
MAAPEPLRPRLCRLVRGEQGYGFHLHGEKGRRGQFIRRVEPGSPAEAAALRAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEESGLHLSPTAAEAKEKARAMRVNKRAPQMDWNRKREIFSNF

SEQ ID NO: 79 - protein, 231 aa, homo sapien
MAQRGLPPAHDPWEPKPDWAHTGSHSSEAGKKDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 80 - protein, 224 aa, homo sapien
MARSGSATPPARAPGAPPRSPPQRLDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 81 - protein, 215 aa, homo sapien
MARSGSATPPARAPGAPPRSPPQRLVQDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEESGLHLSPTAAEAKEKARAMRVNKRAPQMDWNRKREIFSNF

SEQ ID NO: 82 - protein, 226 aa, homo sapien
MARSGSATPPARAPGAPPRSPPQRLVQDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 83 - protein, 450 aa, homo sapien
MAAPEPLRPRLCRLVRGEQGYGFHLHGEKGRRGQFIRRVEPGSPAEAAALAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEARRRLEPCESTSARHRWTGTGSVKSSATSEPLPACLGTLGPLPHGPWASACPELPQPQWTGGWSCHCPEISPSPGEPPSCPCPPGTGGLWQQDRGRETQRCERESETETERERERHRERQRESERARGSRGARAFAALPGPAD

SEQ ID NO: 84 - protein, 122 aa, homo sapien
MWRDCAMLRWWPASRHGRTRPGCWSWTPRQMNTSSGFGSHPPRSTWKVLCRHPSPMEPALPSSMVALRARPEVTCLVPTRTLRRGHRLGCGTRAHSRHPTHRAHPRMAVPGSKIPSRRAAST

SEQ ID NO: 85 - protein, 151 aa, homo sapien
MGTQGAMAQEAAQRRALCLGGGRGAQPRAPERREGEEDCAKPASTAHMGNCLGWLCLTPGNQVVQRIKAVEGQTRLLVVDQETDEELRRRQLTCTEEMAQRGLPPAHDPWEPKPDWAHTGSHSSEAGKKDVSGPLRELRPRLCHLRKGPQG

SEQ ID NO: 86 - protein, 337 aa, homo sapien
MAAPEPLRPRLCRLVRGEQGYGFHLHGEKGRRGQFIRRVEPGSPAEAAALRAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 87 - protein, 326 aa, homo sapien
MAAPEPLRPRLCRLVRGEQGYGFHLHGEKGRRGQFIRRVEPGSPAEAAALRAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEESGLHLSPTAAEAKEKARAMRVNKRAPQMDWNRKREIFSNF

SEQ ID NO: 88 - protein, 231 aa, homo sapien
MAQRGLPPAHDPWEPKPDWAHTGSHSSEAGKKDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 89 - protein, 224 aa, homo sapien
MARSGSATPPARAPGAPPRSPPQRLDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 90 - protein, 215 aa, homo sapien
MARSGSATPPARAPGAPPRSPPQRLVQDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEESGLHLSPTAAEAKEKARAMRVNKRAPQMDWNRKREIFSNF

SEQ ID NO: 91 - protein, 226 aa, homo sapien
MARSGSATPPARAPGAPPRSPPQRLVQDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEAKEKARAMRVNKRAPQMDWNRKREIFSNF

SEQ ID NO: 92 - protein, 450 aa, homo sapien
MAAPEPLRPRLCRLVRGEQGYGFHLHGEKGRRGQFIRRVEPGSPAEAAALAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSWPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEARRRLEPCESTSARHRWTGTGSVKSSATSEPLPACLGTLGPLPHGPWASACPELPQPQWTGGWSCHCPEISPSPGEPPSCPCPPGTGGLWQQDRGRETQRCERESETETERERERHRERQRESERARGSRGARAFAALPGPAD

SEQ ID NO: 93 - DNA, 20 bp, artificial sequence
  Synthetic sequence; gRNA Recognition Sequence
aggttgaacccatagccctg

SEQ ID NO: 94 - DNA, 20 bp, artificial sequence
  Synthetic sequence; gRNA Recognition Sequence
cacctgcgaaagggacctca

SEQ ID NO: 95 - DNA, 20 bp, artificial sequence
  Synthetic sequence; gRNA Recognition Sequence
gtcaacggcgtcaacgtgga

SEQ ID NO: 96 - DNA, 20 bp, artificial sequence
  Synthetic sequence; gRNA Recognition Sequence
ggatgtcagtgggcccctga

SEQ ID NO: 97 - DNA, 20 bp, artificial sequence
  Synthetic sequence; gRNA Recognition Sequence
tgacgccgttgacctcgacc

SEQ ID NO: 98 - DNA, 20 bp, artificial sequence
  Synthetic sequence; gRNA Recognition Sequence
acctgcatagtgacaagtcc

SEQ ID NO: 99 - DNA, 20 bp, artificial sequence
  Synthetic sequence; gRNA Recognition Sequence
tggtggccagcatcaaggca

SEQ ID NO: 100 - DNA, 20 bp, artificial sequence
  Synthetic sequence; gRNA Recognition Sequence
gccgggacttgtcactatgc

SEQ ID NO: 101 - DNA, 20 bp, artificial sequence
  Synthetic sequence; gRNA Recognition Sequence
gtgaacgggcagaatgtgga

SEQ ID NO: 102 - DNA, 20 bp, artificial sequence
  Synthetic sequence; gRNA Recognition Sequence
ttccacctgcacggcgagaa

SEQ ID NO: 103 - DNA, 20 bp, artificial sequence
  Synthetic sequence; gRNA Recognition Sequence
aagccagactgggcacacac

SEQ ID NO: 104 - DNA, 20 bp, artificial sequence
  Synthetic sequence; gRNA Recognition Sequence
cggccagtacatccgctctg

SEQ ID NO: 105 - DNA, 20 bp, artificial sequence
  Synthetic sequence; gRNA Recognition Sequence
tacatccgctctgtggaccc

SEQ ID NO: 106 - DNA, 20 bp, artificial sequence
  Synthetic sequence; gRNA Recognition Sequence
cgaggtcaacggcgtcaacg

SEQ ID NO: 107 - DNA, 20 bp, artificial sequence
  Synthetic sequence; gRNA Recognition Sequence
acagagcggatgtactggcc

SEQ ID NO: 108 - DNA, 20 bp, artificial sequence
  Synthetic sequence; gRNA Recognition Sequence
cgcgccggatgaactgcccg

SEQ ID NO: 109 - DNA, 20 bp, artificial sequence
  Synthetic sequence; gRNA Recognition Sequence
gcggcagctgacctgtaccg

SEQ ID NO: 110 - DNA, 20 bp, artificial sequence
  Synthetic sequence; gRNA Recognition Sequence
gggccggtacctcaatgagc

SEQ ID NO: 111 - DNA, 20 bp, artificial sequence
  Synthetic sequence; gRNA Recognition Sequence
aggctgtggaggggcagact

SEQ ID NO: 112 - DNA, 20 bp, artificial sequence
  Synthetic sequence; gRNA Recognition Sequence
gcagcgcggcggcctcggcg